(19) United States
(12) Patent Application Publication
(10) Pub. No.: US 2022/0276244 A1
(43) Pub. Date: Sep. 1, 2022

(54) CHIMERIC PROTEINS AND METHODS TO SCREEN FOR COMPOUNDS AND LIGANDS BINDING TO GPCRS

(71) Applicant: CONFO THERAPEUTICS N.V., Zwijnaarde (BE)

(72) Inventors: Ann De Blieck, Sinaai (BE); Pieter Claes, Gentbrugge (BE); Cedric Ververken, Merelbeke (BE); Christel Menet, Brussel (BE); Lies Dekeyzer, Knesselare (BE)

(21) Appl. No.: 17/607,334

(22) PCT Filed: Apr. 28, 2020
(86) PCT No.: PCT/EP2020/061802

§ 371 Date: Oct. 28, 2021

Related U.S. Application Data
(60) Provisional application No. 62/840,091, filed on Apr. 29, 2019.
(60) Provisional application No. 62/840,092, filed on Apr. 29, 2019.
(60) Provisional application No. 62/840,094, filed on Apr. 29, 2019.
(60) Provisional application No. 62/863,544, filed on Jun. 19, 2019.
(60) Provisional application No. 62/934,136, filed on Nov. 12, 2019.
(60) Provisional application No. 62/934,181, filed on Nov. 12, 2019.
(60) Provisional application No. 62/934,133, filed on Nov. 12, 2019.

(51) Int. Cl. G01N 33/566; C07K 14/705

(52) U.S. Cl. CPC G01N 33/566; C07K 14/705; C07K 2319/03

(57) ABSTRACT

The invention relates to chimeric GPCRs having extracellular loops from a first GPCR and intracellular loops from a second GPCR, and to screening methods for identifying compounds or ligands that bind to an active conformation of a GPCR in which such chimeric GPCRs are used.

### CROSS-REFERENCE TO RELATED APPLICATIONS

This application is a national phase entry under 35 U.S.C. § 371 of International Patent Application PCT/EP2020/061802, filed Apr. 28, 2020, designating the United States of America and published in English as International Patent Publication WO 2020/221768 on Nov. 5, 2020, which claims the benefit under Article 8 of the Patent Cooperation Treaty to U.S. Provisional Patent Application Ser. No. 62/840,091, filed Apr. 29, 2019, U.S. Provisional Patent Application Ser. No. 62/840,092, filed Apr. 29, 2019, U.S. Provisional Patent Application Ser. No. 62/840,094, filed Apr. 29, 2019, U.S. Provisional Patent Application Ser. No. 62/863,544, filed Jun. 19, 2019, U.S. Provisional Patent Application Ser. No. 62/934,136, filed Nov. 12, 2019, U.S. Provisional Patent Application Ser. No. 62/934,181, filed Nov. 12, 2019, and U.S. Provisional Patent Application Ser. No. 62/934,133, filed Nov. 12, 2019, the entireties of which are hereby incorporated by reference.

The present invention relates to chimeric proteins that can be used to discover and develop compounds and ligands binding to GPCRs.

The invention also relates to methods and arrangements in which said chimeric proteins are used.

In particular, the present invention relates to chimeric proteins, methods and arrangements that can be used to identify compounds and ligands that can bind to GPCRs, and to test and ligands that can bind to GPCRs.

The screening and assay techniques provided by the invention can in particular be used to identify, generate, optimize and/or develop compounds and ligands that can bind to GPCRs and that can be used as and/or developed into therapeutic, prophylactic and diagnostic agents. As further described herein, such compounds or ligands can be any desired and/or suitable compound or ligand, including but not limited to small molecules, small peptides, biological molecules or other chemical entities, and examples of such compounds will be clear to the skilled person based on the further disclosure herein.

For example, small molecules or molecular fragments that are identified and/or generated using the chimeric proteins, methods and arrangements of the invention (i.e. the “hits” from such screening) can be used as a starting point for further drug discovery and development efforts (e.g. using well-known techniques of so-called “hits-to-leads” chemistry), and such further efforts can also involve the use of assays (e.g. a functional assay or an assay used for quality control purposes) in which a chimeric protein of the invention is used. The compounds that are identified using the methods and techniques of the invention (i.e. as “hits”), and any compounds that are generated or developed using such hits as a starting point, are also collectively referred to as herein “compounds of the invention” and form further aspects of the invention. It will be clear to the skilled person that such compounds may for example be so-called “hits”, “leads”, “development candidates”, “pre-clinical compounds”, “clinical candidates” or commercial compounds or products, depending on their stage of development and on the specific terminology that is used by the company or entity that is developing and/or commercializing them.

Also, as further described herein, the methods and assays of the invention may allow allosteric agonists, antagonists and/or inverse agonists to be identified and/or characterized (depending on the specific target and assay used).

Oher features, aspects, embodiments, uses and advantages of the present invention will become clear from the further description herein.

Assay and screening techniques for GPCRs are well-known in the art. It is estimated that over half of all modern medicinal drugs are targeted towards membrane proteins, with roughly a third of all modern medicinal drugs targeting GPCRs. Reference is made to the standard handbooks as well as the further prior art cited herein. [In this respect, it should be noted that generally, within the field, the terms “7TM receptor” and “7TM” are often used interchangeably with “GPCR”, although according to the IUPHAR database, there are some 7TM receptors that do not signal through G proteins. For the purposes of the present description and claims, the terms “GPCR” and “7TM” are used interchangeably herein to include all transmembrane proteins—and in particular transmembrane receptors—with 7-transmembrane domains, irrespective of their intracellular signaling cascade or signal transduction mechanism, although it should be understood that throughout the description and claims, 7TMs that signal through G-proteins are a preferred aspect of the invention].

As is well-known. GPCRs are not static objects whose function is determined solely by their primary, secondary or tertiary structure, but are often flexible structures that can undergo transitions (also referred to as “conformational changes”) between different conformational states, such that a GPCR may exist in an equilibrium between these different states. Some of these states may be functional and/or active, while others may be a basal state (which may or may not exhibit some level of constitutive activity), be an essentially inactive state and/or be a less active state compared to more functional or active states. Also, the geometry of the different epitopes, binding sites (including ligand binding sites) and/or catalytic sites that may exist in or on a GPCR may differ between these different conformations, for example such that in some of the conformational states, a binding site may not be available/accessible for ligand binding and/or such that the affinity for the interaction between the binding site and the relevant ligand(s) is reduced compared to a more active conformational state.

It is also known that binding of a ligand to a GPCR can change its conformation (for example from an inactive/less active conformation into an active/more active conformation) and/or shift its equilibrium from an inactive/less active conformation towards an active/more active conformation. It is also possible that binding of a ligand to one binding site of a GPCR may make another binding site on the GPCR more accessible for its relevant ligand(s) and/or may lead to an increase in the affinity of said other binding site for said ligand(s), and/or shift the equilibrium from a conformation in which said other binding site has less affinity for said ligand(s) towards a conformation in which said other binding site has better affinity for said ligand(s). For example, binding of an extracellular ligand to an extracellular binding site on a GPCR may lead to conformational changes on the cytoplasmic side, which for example may increase the affinity of an intracellular binding site for an intracellular ligand (for example, increase the affinity for the interaction between the G-protein and the G-protein binding site), or visa versa. This change in binding affinity for an intercellular ligand following binding of an extracellular ligand, and the subsequent binding of an intracellular ligand to an intracellular binding site, is part of the mechanism in which a GPCR transducts an extracellular signal.

Generally, as further described herein, it can be said that for GPCRs, an “agonist” of shift the conformational equilibrium from an inactive state (or one or more less active states) towards an active state (or one or more states that are more active), whereas an “inverse agonist” of the GPCR will do the inverse.

Without being limited to any specific hypothesis or mechanism, it is also assumed that a GPCR can form a complex with an extracellular ligand (binding to an extracellular binding site on the GPCR) and an intracellular ligand (binding to an intracellular binding site on the GPCR), and that the interaction between the GPCR and each of the ligands is stabilized by the binding of the other ligand (in other words, that said complex is stabilized by the binding of both ligands). Again, in this case, binding of one or both of the ligands may also shift the conformational equilibrium of the GPCR towards (the formation and/or stabilization of) this complex. Reference is for example made to WO2012/007593 cited below.

Given that the perceived “overall” state of a GPCR is to a large extent governed by the (statistical) distribution of the GPCR over its various possible conformations, and thus by the equilibrium that exists between these conformational states, it should be understood that when, in the present description or claims, a GPCR is said to undergo a conformational change into a certain conformation (i.e. from one or more other conformations), this will include a mechanism or situation where the conformational equilibrium of the GPCR is shifted towards said conformation (i.e. under the specific conditions used, such as the conditions used for screening or the relevant assay). Similarly, when a ligand is said to elicit a conformational change of a GPCR into a certain conformation (i.e. from one or more other conformations), this includes a mechanism or situation where the binding of the ligand shifts the conformational equilibrium of the protein towards said conformation (i.e. under the specific conditions used, such as the conditions used for screening or the relevant assay).

However, it should also be noted that, although any one of the mechanisms described herein (or any combination thereof) may at any given time be involved in the practice of the invention (also depending on, for example, the specific GPCR and/or ligand(s) to which the invention is applied), the invention is its broadest sense is not limited to any specific mechanism, explanation or hypothesis as long as the application of the invention to a specific GPCR results in the technical effect(s) outlined herein.

One of the challenges of screening for compounds that are directed GPCRs is that the correct conformation of the GPCR may be lost if the GPCR is expressed or used in isolation from its native environment (if it is even feasible or possible to express the GPCR and to ensure its proper folding outside of its cellular environment). Also, it may be challenging to ensure that the GPCR is in its desired conformation (often, a functional conformation such as its active conformation) under the conditions that are used for screening. There may also be a need for, or an advantage in achieving, a shift in the conformational equilibrium of the GPCR towards a conformational state that is more suitable for screening or assay purposes (such as an active state or a state in which the relevant binding site is more accessible for, and/or has a geometry that is better for, assay or screening purposes). As further described herein, such a conformation is also referred to as a “druggable” conformation, and according to preferred aspects of the invention, means are applied (as further described herein) to ensure that a druggable format of the intended GPCR is provided.

For example, WO2012/007593, WO2012/007594, WO 2012/175643, WO 2014/118297, WO2014/122183 and WO 2014/118297 are directed towards protein binding domains that can be used to stabilize a particular conformational state of a GPCR for the purposes of determining its structure and for drug screening and discovery purposes. In these references, VHH domains are used that can stabilize the GPCR in a desired conformation, and in particular a (more) druggable conformation, such as a functional state and/or active state, for example in the conformation that arises when an activating ligand (agonist) binds to the extracellular side of the GPCR so as to allow the GPCR to activate heterotrimeric G proteins. Reference is for example also made to Pardon et al., Angew Chem Int Ed Engl. 2018, 57(19):5292-5295; Che et al., Cell. 2018, 172 (1-2):55-67; Manglik et al., Annu Rev Pharmacol Toxicol. 2017; 57:19-37; Pardon et al., Nat Protoc. 2014, 674-93; Kruse et al., Nature. 2013, 504(7478); Steyaert and Kobilka, Curr Opin Struct Biol. 2011, 567-72; Eglen and Reisine, Pharmaceuticals 2011, 4, 244-272; and Rasmussen et al., Nature. 2011, 469(7329): 175-180 and the further references cited therein. VHH domains that can be used to stabilize a desired conformation of a membrane protein such as a GPCR are also referred to herein as ConfoBodies [Confobody™ is a registered trademark of Confo Therapeutics, Ghent, Belgium].

Some specific, but non-limiting examples of ConfoBodies that can bind to an intracellular epitope of a GPCR and that can be used to stabilize a GPCR in a desired conformation (and that can also be used in the present invention) are the VHH called CA2764, CA3431, CA3413, CA2780, CA2765, CA2761, CA3475, CA2770, CA3472, CA3420, CA3433, CA3434, CA3484, CA2760, CA2773, CA3477, CA2774, CA2768, CA3424, CA2767, CA2786, CA3422, CA2763, CA2772, CA2771, CA2769, CA2782, CA2783 and CA2784 (see for example WO 2012/007593, Tables 1 and 2 and SEQ ID NO's: 1 to 29); the VHH called CA5669, Nb9-1, Nb9-8, XA8633 and CA4910 (see for example WO 2014/118297, Tables 1 and 2 and SEQ ID NO's: 15, 16, 17, 19 and 20); the VHH called Nb9-11, Nb9-7, Nb9-7, Nb9-22, Nb9-17, Nb9-24, Nb9-9, Nb9-14, Nb9-2, Nb9-20, Nb_C3, NbH-4, Nb-E1, Nb_A2, Nb_B4, Nb_D3, Nb_D1 and Nb_H1 (see for example WO 2014/122183, Tables 1 and 2 and SEQ ID NOs: 1-19); and the VHH called XA8639, XA8635, XA8727 and XA9644 (see for example WO 2015/121092, Tables 2 and 3 and SEQ ID NOs: 2 to 6 and 74).

Some specific, but non-limiting examples of VHH that can bind to a G-protein are CA4435, CA4433, CA4436, CA4437, CA4440 and CA4441 (see for example WO 2012/175643007593, Tables 2 and 3 and SEQ ID NO's: 1 to 6).

Generally, the methods described in said prior art to raise such a VHHs require that a desired GPCR can provided and used in a suitable conformation (i.e. the conformation against which the VHHs are to be raised). This is not only the case for purposes of immunization (i.e. to generate an immune library), but also for the purposes of selection and screening (which will require proper expression of the desired conformation of the GPCR on a phage, ribosome or other display system used for screening the immune library), and also to screening and selection using naïve libraries or synthetic libraries. If these limitations result in a situation where no suitable VHHs can be obtained against the desired conformation(s) of a GPCR, it may be that these prior art methods may be of limited use when they are to be applied to said GPCR.

Generally, the present invention aims to offer an alternative methodology for providing assay techniques and compound/ligand screening methods that can be used with GPCRs. In particular, the invention aims to provide such methodology that avoids the need to generate VHHs that are specific for the desired conformation of the native GPCR, and thus avoid any difficulties or limitations that may be associated therewith.

More in particular, the invention aims to provide combinations of GPCRs and VHH directed towards said GPCRs that can be used in assay and screening techniques.

The assay and screening techniques that are provided by the invention can be used to discover and develop (e.g. to identify, generate, test and optimize) compounds that are directed towards the relevant GPCR (i.e. that have specificity for one or more GPCRs and/or that are intended to target one or more GPCRs, e.g. for therapeutic, prophylactic and/or diagnostic purposes). Preferably, such compounds will be specific for one particular GPCR compared to other (closely related) GPCRs (i.e. will be selective for one particular GPCR).

The compounds identified and/or developed using the assays and screening techniques provided by the invention can be used to modulate (as defined herein) the relevant GPCR, its signaling and/or the biological functions, pathways and/or mechanisms in which said GPCR or its signaling is involved. For example, the invention can be used to discover and develop compounds that are agonists, antagonists, inverse agonists, inhibitors or modulators (such as allosteric modulators) of the GPCR and/or of the signaling, the pathway and/or the physiological and/or biological mechanisms in which the GPCR is involved.

Usually, the compounds that are discovered and/or developed using the invention will be directed towards GPCR that is expressed by or on a cell that is present in the body of a subject that is to be treated with a compound or ligand that has been discovered or developed using the methods and techniques of the invention.

The invention can be used to discover and/or develop any kind of compound that is suitable for its intended use, which will often be a use as a therapeutic, diagnostic or prophylactic agent. As such, these compounds may be small molecules, peptides, biological molecules or other chemical entities. Examples of suitable biological molecules may for example include antibodies and antibody fragments (such as Fabs, VH, VL and VHH domains) and compounds based on antibody fragments (such as ScFvs and diabodies and other compounds or constructs comprising one or more VH, VL and/or VHH domains), compounds based on other protein scaffolds such Alphabodies™ and scaffolds based on avimers, PDZ domains, protein A domains (such as Affibodies™), ankyrin repeats (such as DARPins™), fibronectin (such as Adnectins™) and lipocalins (such as Anticalins™) as well as binding moieties based on DNA or RNA including but not limited to DNA or RNA aptamers. Reference is made to the further description herein, as well as for example to Simeon and Chen, Protein Cell 2018, 9(1): 3-14, Binz et al, Nat. Biotech 2005, Vol 23: 1257 and Ulrich et al., Comb Chem High Throughput Screen 2006 9(8):619-32.

The methods and techniques of the invention can for example be used to screen libraries of such compounds in order to identify one or more “hits” that are specific for the relevant GPCR (and in particular for a desired conformation of the GPCR and/or that are capable of inducing a desired conformation of the GPCR, such as a ligand-bound and in particular agonist-bound conformation) and/or as an assay that is used as part of a strategy to improve the affinity and/or potency of compounds that are directed against a GPCR and/or to otherwise improve (the pharmacological and/or other properties of) such a compound (for example, in the case of a small molecule, as part of a “hits-to-leads” campaign).

The chimeric proteins, methods and techniques of the invention can also be used for the purposes of so-called “fragment-based drug discovery” or “FBDD” (also known as “fragment-based lead discovery” or “FBLD”). Reference is for example made to Lamoree and Hubbard, Essays in Biochemistry (2017) 61, 453-464, and standard handbooks such as Jahnke and Erlanson, “Fragment-based approaches in drug discovery”, 2006; Zartler and Shapiro, “Fragment-based drug discovery: a practical approach”, 2008; and Kuo “Fragment based drug design: tools, practical approaches, and examples”, 2011.

The present invention will be described herein with respect to particular embodiments and with reference to certain non-limiting examples and figures. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Unless otherwise defined herein, scientific and technical terms and phrases used in connection with the present invention shall have the meanings that are commonly understood by those of ordinary skill in the art. Generally, nomenclatures used in connection with, and techniques of molecular and cellular biology, structural biology, biophysics, pharmacology, genetics and protein and nucleic acid chemistry described herein are those well-known and commonly used in the art. Singleton, et al., Dictionary of Microbiology and Molecular Biology, 2D ED., John Wiley and Sons, New York (1994), and Hale & Marham, The Harper Collins Dictionary of Biology, Harper Perennial, NY (1991) provide one of skill with general dictionaries of many of the terms used in this disclosure. The methods and techniques of the present invention are generally performed according to conventional methods well known in the art and as described in various general and more specific references that are cited and discussed throughout the present specification unless otherwise indicated. See, for example, Sambrook et al. Molecular Cloning: A Laboratory Manual, 3th ed., Cold Spring Harbor Laboratory Press, Cold Spring Harbor, N.Y. (2001); Ausubel et al., Current Protocols in Molecular Biology, Greene Publishing Associates (1992, and Supplements to 2002); up, Biomolecular crystallography: principles, Practice and Applications to Structural Biology, 1st edition, Garland Science, Taylor & Francis Group, LLC, an informa Business, N.Y. (2009); Limbird, Cell Surface Receptors, 3d ed., Springer (2004).

As used herein, the terms “polypeptide”, “protein”, “peptide” are used interchangeably herein, and refer to a polymeric form of amino acids of any length, which can include coded and non-coded amino acids, chemically or biochemically modified or derivatized amino acids, and polypeptides having modified peptide backbones. Throughout the application, the standard one letter notation of amino acids will be used. Typically, the term “amino acid” will refer to “proteinogenic amino acid”, i.e. those amino acids that are naturally present in proteins. Most particularly, the amino acids are in the L isomeric form, but D amino acids are also envisaged.

As used herein, the terms “nucleic acid molecule”, “polynucleotide”, “polynucleic acid”, “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

Any of the peptides, polypeptides, nucleic acids, compound, etc., disclosed herein may be “isolated” or “purified”. “Isolated” is used herein to indicate that the material referred to is (i) separated from one or more substances with which it exists in nature (e.g., is separated from at least some cellular material, separated from other polypeptides, separated from its natural sequence context), and/or (ii) is produced by a process that involves the hand of man such as recombinant DNA technology, protein engineering, chemical synthesis, etc.; and/or (iii) has a sequence, structure, or chemical composition not found in nature. “Isolated” is meant to include compounds that are within samples that are substantially enriched for the compound of interest and/or in which the compound of interest is partially or substantially purified. “Purified” as used herein denote that the material referred to is removed from its natural environment and is at least 60% free, at least 75% free, or at least 90% free from other components with which it is naturally associated, also referred to as being “substantially pure”.

The term “sequence identity” as used herein refers to the extent that sequences are identical on a nucleotide-by-nucleotide basis or an amino acid-by-amino acid basis over a window of comparison.

Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Determining the percentage of sequence identity can be done manually, or by making use of computer programs that are available in the art. Examples of useful algorithms are PILEUP (Higgins & Sharp, CABIOS 5:151 (1989), BLAST and BLAST 2.0 (Altschul et al. J. Mol. Biol. 215: 403 (1990). Software for performing BLAST analyses is publicly available through the National Center for Biotechnology Information (http://www.ncbi.nlm.nih.gov/).

“Similarity” refers to the percentage number of amino acids that are identical or constitute conservative substitutions. Similarity may be determined using sequence comparison programs such as GAP (Deveraux et al. 1984). In this way, sequences of a similar or substantially different length to those cited herein might be compared by insertion of gaps into the alignment, such gaps being determined, for example, by the comparison algorithm used by GAP. As used herein, “conservative substitution” is the substitution of amino acids with other amino acids whose side chains have similar biochemical properties (e.g. are aliphatic, are aromatic, are positively charged, . . . ) and is well known to the skilled person. Non-conservative substitution is then the substitution of amino acids with other amino acids whose side chains do not have similar biochemical properties (e.g. replacement of a hydrophobic with a polar residue). Conservative substitutions will typically yield sequences which are not identical anymore, but still highly similar. By conservative substitutions is intended combinations such as gly, ala; val, ile, leu, met; asp, glu; asn, gin; ser, thr; lys, arg; cys, met; and phe, tyr, trp.

A “deletion” is defined here as a change in either an amino acid or nucleotide sequence in which one or more amino acid or nucleotide residues, respectively, are absent as compared to an amino acid sequence or nucleotide sequence of a parental polypeptide or nucleic acid. Within the context of a protein, a deletion can involve deletion of about 2, about 5, about 10, up to about 20, up to about 30 or up to about 50 or more amino acids. A protein or a fragment thereof may contain more than one deletion. Within the context of a GPCR, a deletion may also be a loop deletion, or an N- and/or C-terminal deletion. As will be clear to the skilled person, an N- and/or C-terminal deletion of a GPCR is also referred to as a truncation of the amino acid sequence of the GPCR or a truncated GPCR.

An “insertion” or “addition” is that change in an amino acid or nucleotide sequence which has resulted in the addition of one or more amino acid or nucleotide residues, respectively, as compared to an amino acid sequence or nucleotide sequence of a parental protein. “Insertion” generally refers to addition to one or more amino acid residues within an amino acid sequence of a polypeptide, while “addition” can be an insertion or refer to amino acid residues added at an N- or C-terminus, or both termini. Within the context of a protein or a fragment thereof, an insertion or addition is usually of about 1, about 3, about 5, about 10, up to about 20, up to about 30 or up to about 50 or more amino acids. A protein or fragment thereof may contain more than one insertion.

A “substitution”, as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. By conservative substitutions is intended combinations such as gly, ala; val, ile, leu, met; asp, glu; asn, gin; ser, thr; lys, arg; cys, met; and phe, tyr, trp.

A “mutation” is defined herein as a change in either an amino acid or nucleotide sequence that is a deletion, insertion or substitution as described herein. When an amino acid or nucleotide sequence contains two or more such mutations, each of these mutations may independently be a deletion, insertion or substitution.

The term “amino acid differences” refers to the total number of amino acid residues in a sequence that have been changed (i.e. by substitution, insertion and/or deletion) compared to a starting or reference sequence. The number of amino acid differences between a sequence and a reference sequence can usually be determined by comparing these sequences, e.g. by making an alignment.

The term “ortholog” when used in reference to an amino acid or nucleotide/nucleic acid sequence from a given species refers to the same amino acid or nucleotide/nucleic acid sequence from a different species. It should be understood that two sequences are orthologs of each other when they are derived from a common ancestor sequence via linear descent and/or are otherwise closely related in terms of both their sequence and their biological function. Orthologs will usually have a high degree of sequence identity but may not (and often will not) share 100% sequence identity.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express nucleic acids or polypeptides that are not found within the native (non-recombinant) form of the cell or express native genes that are otherwise abnormally expressed, under expressed, over expressed or not expressed at all.

As used herein, the term “expression” refers to the process by which a polypeptide is produced based on the nucleic acid sequence of a gene. The process includes both transcription and translation.

The term “operably linked” as used herein refers to a linkage in which the regulatory sequence is contiguous with the gene of interest to control the gene of interest, as well as regulatory sequences that act in trans or at a distance to control the gene of interest. For example, a DNA sequence is operably linked to a promoter when it is ligated to the promoter downstream with respect to the transcription initiation site of the promoter and allows transcription elongation to proceed through the DNA sequence. A DNA for a signal sequence is operably linked to DNA coding for a polypeptide if it is expressed as a pre-protein that participates in the transport of the polypeptide. Linkage of DNA sequences to regulatory sequences is typically accomplished by ligation at suitable restriction sites or adapters or linkers inserted in lieu thereof using restriction endonucleases known to one of skill in the art.

The term “regulatory sequence” as used herein, and also referred to as “control sequence”, refers to polynucleotide sequences which are necessary to affect the expression of coding sequences to which they are operably linked. Regulatory sequences are sequences which control the transcription, post-transcriptional events and translation of nucleic acid sequences. Regulatory sequences include appropriate transcription initiation, termination, promoter and enhancer sequences; efficient RNA processing signals such as splicing and polyadenylation signals; sequences that stabilize cytoplasmic mRMA; sequences that enhance translation efficiency (e.g., ribosome binding sites); sequences that enhance protein stability; and when desired, sequences that enhance protein secretion. The nature of such control sequences differs depending upon the host organism. The term “regulatory sequence” is intended to include, at a minimum, all components whose presence is essential for expression, and can also include additional components whose presence is advantageous, for example, leader sequences and fusion partner sequences.

The term “vector” as used herein is intended to refer to a nucleic acid molecule capable of transporting another nucleic acid molecule to which it has been linked. The vector may be of any suitable type including, but not limited to, a phage, virus, plasmid, phagemid, cosmid, bacmid or even an artificial chromosome. Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of certain genes of interest. Such vectors are referred to herein as “recombinant expression vectors” (or simply, “expression vectors”). Suitable vectors have regulatory sequences, such as promoters, enhancers, terminator sequences, and the like as desired and according to a particular host organism (e.g. bacterial cell, yeast cell). Typically, a recombinant vector according to the present invention comprises at least one “chimeric gene” or “expression cassette”. Expression cassettes are generally DNA constructs preferably including (5′ to 3′ in the direction of transcription): a promoter region, a polynucleotide sequence, homologue, variant or fragment thereof of the present invention operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The term “host cell”, as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may be an isolated cell or cell line grown in culture or may be a cell which resides in a living tissue or organism. In particular, host cells are of bacterial or fungal origin, but may also be of plant or mammalian origin. The wordings “host cell”, “recombinant host cell”, “expression host cell”, “expression host system”, “expression system”, are intended to have the same meaning and are used interchangeably herein.

In the present invention, as is common in the art, amino acid sequences are given using the one-letter amino acid code starting at the N-terminal end and ending at the C-terminal end. Also, in the present description and claims, a position or residue is said to be “upstream” of a given position or residue if said first mentioned position or residue is closer to the N-terminal end than the given position or residue, and “downstream” if said first mentioned position or residue is closer to the C-terminal end than the given position or residue.

“G-protein coupled receptors” or “GPCRs” are polypeptides that share a common structural motif, having an extracellular amino-terminus (N-terminus), an intracellular carboxy terminus (C-terminus) and seven hydrophobic transmembrane seven regions of between 22 to 24 hydrophobic amino acids that form seven alpha helices, each of which spans a membrane. Each span is identified by number, i.e., transmembrane-1 (TM1), transmembrane-2 (TM2), etc. The transmembrane helices are joined by regions of amino acids between transmembrane-2 and transmembrane-3, transmembrane-4 and transmembrane-5, and transmembrane-6 and transmembrane-7 on the exterior, or “extracellular” side, of the cell membrane, referred to as “extracellular” regions 1, 2 and 3 (EC1, EC2 and EC3), respectively. The transmembrane helices are also joined by regions of amino acids between transmembrane-1 and transmembrane-2, transmembrane-3 and transmembrane-4, and transmembrane-5 and transmembrane-6 on the interior, or “intracellular” side, of the cell membrane, referred to as “intracellular” regions 1, 2 and 3 (IC1, IC2 and IC3), respectively. The “carboxy” (“C”) terminus of the receptor lies in the intracellular space within the cell, and the “amino” (“N”) terminus of the receptor lies in the extracellular space outside of the cell. GPCR structure and classification is generally well known in the art, and further discussion of GPCRs may be found in Cvicek et al., PLoS Comput Biol. Mar. 30, 2016; 12 (3):e1004805. doi: 10.1371/journal.pcbi.1004805; Ventakakrishnan, Current Opinion in Structural Biology, 2014, 27:129-137; Isberg, Trends Pharmacol. Sci., 2015 January, 22-13, Probst, DNA Cell Biol. 1992 11:1-20; Marchese et al Genomics 23: 609-618, 1994; and the following books: Jurgen Wess (Ed) Structure-Function Analysis of G Protein-Coupled Receptors published by Wiley Liss (1st edition; Oct. 15, 1999); Kevin R. Lynch (Ed) Identification and Expression of G Protein-Coupled Receptors published by John Wiley & Sons (March 1998) and Tatsuya Haga (Ed), G Protein-Coupled Receptors, published by CRC Press (Sep. 24, 1999); and Steve Watson (Ed) G-Protein Linked Receptor Factsbook, published by Academic Press (1st edition; 1994). As described in the said prior art and other scientific literature, in a naturally occurring GPCR, the N- and C-terminal parts, the TM domains, the intracellular loops and the extracellular loops are usually arranged as follows (from the N-terminal end to the C-terminal end):

[N-terminal sequence]-[TM1]-[IC1]-[TM2]-[EC1]-[TM3]-[IC2]-[TM4]-[EC2]-[TM5]-[IC3]-[TM6]-[EC3]-[TM7]-[C-terminal sequence].

The International Union of Basic and Clinical Pharmacology (IUPHAR) maintains a database (http://www.guidetopharmacology.org/targets.jsp) of receptors (including GPCRs) and their known endogenous ligands and signaling mechanisms. According to this database, as of January 2019, about 800 GPCRs have been identified in man, of which about half have sensory functions (for example olfaction, taste, light perception and pheromone signaling) and about half mediate signaling associated with ligands that range in size from small molecules to peptides to large proteins. The IUPHAR database as of January 2019 describes two systems for classifying GPCRs, one of which is based on six classes of GPCRs, as follows: Class A (rhodopsin-like), Class B (secretin receptor family), Class C (metabotropic glutamate), Class D (fungal mating pheromone receptors, not found in vertebrates), Class E (cyclic AMP receptors, also not found in vertebrates) and Class F (frizzled/smoothened). The IUPHAR database also mentions an alternative classification scheme known as “GRAFS” which divides the vertebrate GPCRs into five classes (overlapping with the A-F nomenclature), as follows: the Glutamate family (overlapping with the above “class C”), which inter alia includes metabotropic glutamate receptors, a calcium-sensing receptor and GABAB receptors; the Rhodopsin family (overlapping with the above “class A”), which includes receptors for a wide variety of small molecules, neurotransmitters, peptides and hormones, together with olfactory receptors, visual pigments, taste type 2 receptors and five pheromone receptors (V1 receptors); the Adhesion family GPCRs (which are phylogenetically related to class B receptors); the Frizzled family, consisting of 10 Frizzled proteins (FZD(1-10)) and Smoothened (SMO); and the Secretin family, which are receptors for peptide ligands/hormones having between 27-141 amino acid residues, including glucagon, glucagon-like peptides (GLP-1, GLP-2), glucose-dependent insulinotropic polypeptide (GIP), secretin, vasoactive intestinal peptide (VIP), pituitary adenylate cyclase-activating polypeptide (PACAP) and growth-hormone-releasing hormone (GHRH). In this description and in the appended claims, the Type A-to-F classification will be used, unless explicitly stated otherwise. Further reference is made to Cvicek et al., cited herein.

The term “biologically active”, with respect to a GPCR, refers to a GPCR having a biochemical function (e.g., a binding function, a signal transduction function, or an ability to change conformation as a result of ligand binding) of a naturally occurring GPCR.

In general, the term “naturally-occurring” in reference to a GPCR means a GPCR that is naturally produced (e.g., by a wild type mammal such as a human). Such GPCRs are found in nature. The term “non-naturally occurring” in reference to a GPCR means a GPCR that is not naturally-occurring. Naturally-occurring GPCRs that have been made constitutively active through mutation, and variants of naturally-occurring transmembrane receptors, e.g., epitope-tagged GPCRs and GPCRs lacking their native N-terminus are examples of non-naturally occurring GPCRs. Non-naturally occurring versions of a naturally occurring GPCR are often activated by the same ligand as the naturally-occurring GPCR. Non-limiting examples of either naturally-occurring or non-naturally occurring GPCRs within the context of the present invention are provided further herein.

An “epitope”, as used herein, refers to an antigenic determinant of a polypeptide. An epitope could comprise 3 amino acids in a spatial conformation, which is unique to the epitope. Generally an epitope consists of at least 4, 5, 6, 7 such amino acids, and more usually, consists of at least 8, 9, 10 such amino acids. Methods of determining the spatial conformation of amino acids are known in the art, and include, for example, x-ray crystallography and multi-dimensional nuclear magnetic resonance. A “conformational epitope”, as used herein, refers to an epitope comprising amino acids in a spacial conformation that is unique to a folded 3-dimensional conformation of the polypeptide. Generally, a conformational epitope consists of amino acids that are discontinuous in the linear sequence that come together in the folded structure of the protein. However, a conformational epitope may also consist of a linear sequence of amino acids that adopts a conformation that is unique to a folded 3-dimensional conformation of the polypeptide (and not present in a denatured state).

The term “conformation” or “conformational state” of a protein (such as a GPCR) refers generally to a spacial arrangement, structure or range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., a-helix, β-sheet, among others), tertiary structure (e.g., the three dimensional folding of a polypeptide chain), and quaternary structure (e.g., interactions of a polypeptide chain with other protein subunits). Post-translational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

A “functional conformation” or a “functional conformational state”, as used herein, refers to the fact that proteins (such as a GPCRs) possess different conformational states having a dynamic range of activity, in particular ranging from no activity to maximal activity. It should be clear that “a functional conformational state” is meant to cover any conformational state of a protein, having any activity, including no activity, and is not meant to cover the denatured states of proteins. Non-limiting examples of functional conformations include active conformations, inactive conformations or basal conformations (as defined further herein). As mentioned, a particular class of functional conformations is defined as “druggable conformation” and generally refers to the therapeutically relevant conformational state(s) of the protein. Reference is for example made to Johnson and Karanicolas, PLoS Comput Biol 9(3): e1002951. doi:10.1371/journal.pcbi.1002951 and to for example WO2014/122183 which describes that the agonist-bound active conformation of the muscarinic acetylcholine receptor M2 corresponds to the druggable conformation of this receptor relating to pain and gliobastoma, and describes VHHs that can stabilize said druggable conformation for assay and screening purposes. It will thus be understood that druggability is confined to particular conformations depending on the therapeutic indication. More details are provided further herein.

With respect to a protein that is a receptor (such as a GPCR), the term “active conformation”, as used herein, more specifically refers to a conformation or spectrum of receptor conformations that allows signal transduction towards an intracellular effector system, such as G protein dependent signaling and/or G protein-independent signaling (e.g. β-arrestin signaling). An “active conformation” thus encompasses a range of ligand-specific conformations, including an agonist-specific active state conformation, a partial agonist-specific active state conformation or a biased agonist-specific active state conformation, so that it induces the cooperative binding of an intracellular effector protein.

In addition to the foregoing, with respect to a GPCR, the terms “active conformation” and “active form” as used herein refer to a GPCR that is folded in a way so as to be (functionally) active. A GPCR can be placed into an active conformation using an activating ligand (agonist) of the receptor, and such a conformational change will generally enable the receptor to activate heterotrimeric G proteins. For example, a GPCR in its active conformation binds to heterotrimeric G protein and catalyzes nucleotide exchange of the G-protein to activate downstream signaling pathways. Activated GPCRs bind to the inactive, GDP-bound form of heterotrimeric G-proteins and cause the G-proteins to release their GDP so GTP can bind. There is a transient ‘nucleotide-free’ state that results from this process that enables GTP to bind. Once GTP is bound, the receptor and G-protein dissociate, allowing the GTP-bound G protein to activate downstream signaling pathways such as adenylyl cyclase, ion channels, RAS/MAPK, etc. The terms “inactive conformation” and “inactive form” refer to a GPCR that is folded in a way so as to be inactive. A GPCR can be placed into an inactive conformation using an inverse agonist of the receptor. For example, a GPCR in its inactive conformation does not bind to heterotrimeric G protein with high affinity. The terms “active conformation” and “inactive conformation” will be illustrated further herein. As used herein, the term “basal conformation” refers to a GPCR that is folded in a way that it exhibits activity towards a specific signaling pathway even in the absence of an agonist (also referred to as basal activity or constitutive activity). Inverse agonists can inhibit this basal activity. Thus, a basal conformation of a GPCR corresponds to a stable conformation or prominent structural species in the absence of ligands or accessory proteins.

Similarly, with respect to a protein that is a receptor (such as a GPCR), the term “inactive conformation” as used herein refers to a spectrum of receptor conformations that does not allow or blocks signal transduction towards an intracellular effector system. An “inactive conformation” thus encompasses a range of ligand-specific conformations, including an inverse agonist-specific inactive state conformation, so that it prevents the cooperative binding of an intracellular effector protein. It will be understood that the site of binding of the ligand is not critical for obtaining an active or inactive conformation. Hence, orthosteric ligands as well as allosteric modulators may equally be capable of stabilizing a receptor in an active or inactive conformation.

The term “binding agent”, as used herein, means the whole or part of a proteinaceous (protein, protein-like or protein containing) molecule that is capable of binding using specific intermolecular interactions to a membrane protein (such as a GPCR). In a particular embodiment, the term “binding agent” is not meant to include a naturally-occurring binding partner of the relevant membrane protein, such as a G protein, an arrestin, an endogenous ligand; or variants or derivatives (including fragments) thereof. More specifically, the term “binding agent” refers to a polypeptide, more particularly a protein domain. A suitable protein domain is an element of overall protein structure that is self-stabilizing and that folds independently of the rest of the protein chain and is often referred to as “binding domain”. Such binding domains vary in length from between about 25 amino acids up to 500 amino acids and more. Many binding domains can be classified into folds and are recognizable, identifiable, 3-D structures. Some folds are so common in many different proteins that they are given special names. Non-limiting examples are binding domains selected from a 3- or 4-helix bundle, an armadillo repeat domain, a leucine-rich repeat domain, a PDZ domain, a SUMO or SUMO-like domain, a cadherin domain, an immunoglobulin-like domain, phosphotyrosine-binding domain, pleckstrin homology domain, src homology 2 domain, amongst others. A binding domain can thus be derived from a naturally occurring molecule, e.g. from components of the innate or adaptive immune system, or it can be entirely artificially designed.

In general, a binding domain can be immunoglobulin-based or it can be based on domains present in proteins, including but limited to microbial proteins, protease inhibitors, toxins, fibronectin, lipocalins, single chain antiparallel coiled coil proteins or repeat motif proteins. Particular examples of binding domains which are known in the art include, but are not limited to: antibodies, heavy chain antibodies (hcAb), single domain antibodies (sdAb), minibodies, the variable domain derived from camelid heavy chain antibodies (VHH or Nanobodies), the variable domain of the new antigen receptors derived from shark antibodies (VNA), alphabodies, protein A, protein G, designed ankyrin-repeat domains (DARPins), fibronectin type III repeats, anticalins, knottins, engineered CH2 domains (nanoantibodies), engineered SH3 domains, affibodies, peptides and proteins, lipopeptides (e.g. pepducins) (see, e.g., Gebauer & Skerra, 2009; Skerra, 2000; Starovasnik et al., 1997; Binz et al., 2004; Koide et al., 1998; Dimitrov, 2009; Nygren et al. 2008; WO2010066740). Frequently, when generating a particular type of binding domain using selection methods, combinatorial libraries comprising a consensus or framework sequence containing randomized potential interaction residues are used to screen for binding to a molecule of interest, such as a protein.

According to a preferred embodiment, it is particularly envisaged that the binding agent of the invention is derived from an innate or adaptive immune system. Preferably, said binding agent is derived from an immunoglobulin. Preferably, the binding agent according to the invention is derived from an antibody or an antibody fragment. The term “antibody” (Ab) refers generally to a polypeptide encoded by an immunoglobulin gene, or a functional fragment thereof, that specifically binds and recognizes an antigen, and is known to the person skilled in the art. An antibody is meant to include a conventional four-chain immunoglobulin, comprising two identical pairs of polypeptide chains, each pair having one “light” (about 25 kDa) and one “heavy” chain (about 50 kDa). Typically, in conventional immunoglobulins, a heavy chain variable domain (VH) and a light chain variable domain (VL) interact to form an antigen binding site. The term “antibody” is meant to include whole antibodies, including single-chain whole antibodies, and antigen-binding fragments. In some embodiments, antigen-binding fragments may be antigen-binding antibody fragments that include, but are not limited to, Fab, Fab′ and F(ab′)2, Fd, single-chain Fvs (scFv), single-chain antibodies, disulfide-linked Fvs (dsFv) and fragments comprising or consisting of either a VL or VH domain, and any combination of those or any other functional portion of an immunoglobulin peptide capable of binding to the target antigen. The term “antibodies” is also meant to include heavy chain antibodies, or fragments thereof, including immunoglobulin single variable domains, as defined further herein.

The term “immunoglobulin single variable domain” or “ISVD” defines molecules wherein the antigen binding site is present on, and formed by, a single immunoglobulin domain (which is different from conventional immunoglobulins or their fragments, wherein typically two immunoglobulin variable domains interact to form an antigen binding site). It should however be clear that the term “immunoglobulin single variable domain” does comprise fragments of conventional immunoglobulins wherein the antigen binding site is formed by a single variable domain. Preferably, the binding agent within the scope of the present invention is an immunoglobulin single variable domain.

Generally, an immunoglobulin single variable domain will be an amino acid sequence comprising 4 framework regions (FR1 to FR4) and 3 complementary determining regions (CDR1 to CDR3), preferably according to the following formula (1): FR1-CDR1-FR2-CDR2-FR3-CDR3-FR4 (1), or any suitable fragment thereof (which will then usually contain at least some of the amino acid residues that form at least one of the complementarity determining regions). ISVDs comprising 4 FRs and 3 CDRs are known to the person skilled in the art and have been described, as a non-limiting example, in Wesolowski et al. 2009. Typical, but non-limiting, examples of immunoglobulin single variable domains include light chain variable domain sequences (e.g. a VL domain sequence) or a suitable fragment thereof, or heavy chain variable domain sequences (e.g. a VH domain sequence or VHH domain sequence) or a suitable fragment thereof, as long as it is capable of forming a single antigen binding unit. Thus, according to a preferred embodiment, the binding agent is an immunoglobulin single variable domain that is a light chain variable domain sequence (e.g. a VL domain sequence) or a heavy chain variable domain sequence (e.g. a VH domain sequence); more specifically, the immunoglobulin single variable domain is a heavy chain variable domain sequence that is derived from a conventional four-chain antibody or a heavy chain variable domain sequence that is derived from a heavy chain antibody. The immunoglobulin single variable domain may be a domain antibody, or a single domain antibody, or a “dAB” or dAb, or a Nanobody (as defined herein), or another immunoglobulin single variable domain, or any suitable fragment of any one thereof. For a general description of single domain antibodies, reference is made to the following book: “Single domain antibodies”, Methods in Molecular Biology, Eds. Saerens and Muyldermans, 2012, Vol 911. The immunoglobulin single variable domains, generally comprise a single amino acid chain that can be considered to comprise 4 “framework sequences” or FR's and 3 “complementary determining regions” or CDR's (as defined hereinbefore). It should be clear that framework regions of immunoglobulin single variable domains may also contribute to the binding of their antigens (Desmyter et al 2002; Korotkov et al. 2009).

As further described herein, the total number of amino acid residues in a VHH, Nanobody or ConfoBody can be in the region of 110-120, is preferably 112-115, and is most preferably 113. It should however be noted that parts, fragments, analogs or derivatives (as further described herein) of a VHH or Nanobody are not particularly limited as to their length and/or size, as long as such parts, fragments, analogs or derivatives meet the further requirements outlined herein and are also preferably suitable for the purposes described herein.

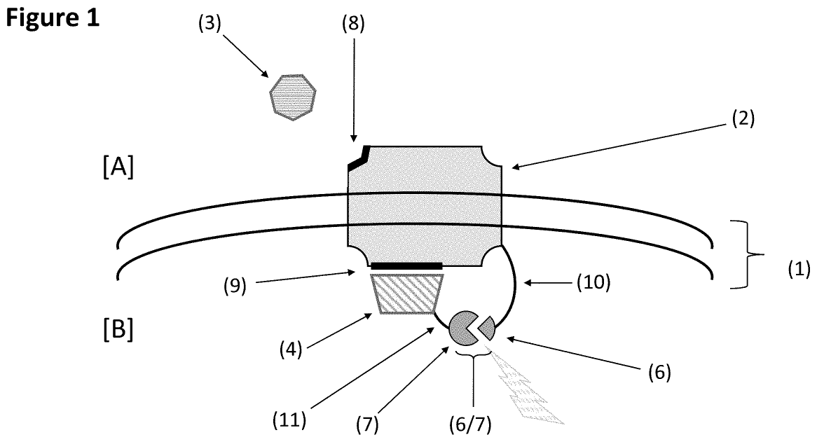

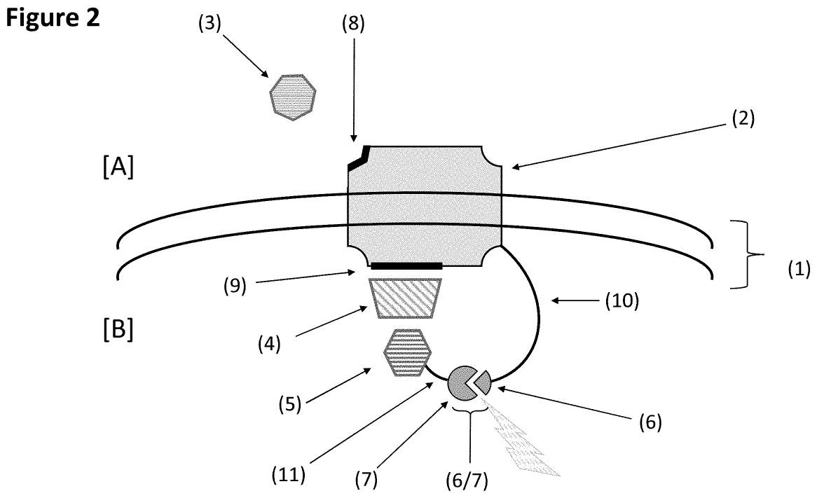

In the present application, the amino acid residues/positions in an immunoglobulin heavy-chain variable domain will be indicated with the numbering according to Kabat (“Sequence of proteins of immunological interest”, US Public Health Services, NIH Bethesda, Md., Publication No. 91), as applied to V_{HH} domains from Camelids in the article of Riechmann and Muyldermans, J. Immunol. Methods Jun. 23, 2000; 240 (1-2): 185-195 (see for example FIG. 2 of this publication). Reference is for example also made to FIG. 1 of the International application WO 2108/134235, which gives a table listing some of the amino acid positions in a VHH and their numbering according to some alternative numbering systems (such as Aho and IMGT. Note: unless explicitly indicated otherwise, for the present description and claims, Kabat numbering is decisive for the amino acid residues/positions in VHH, Nanobody or ConfoBody; other numbering systems are given for reference only).

With regard to the CDR's, as is well-known in the art, there are multiple conventions to define and describe the CDR's of a VH or VHH fragment, such as the Kabat definition (which is based on sequence variability and is the most commonly used) and the Chothia definition (which is based on the location of the structural loop regions). Reference is for example made to the website http://www.bioinf.org.uk/abs/). For the purposes of the present specification and claims, even though the CDR's according to Kabat may also be mentioned, the CDRs are most preferably defined on the basis of the Abm definition (which is based on Oxford Molecular's AbM antibody modelling software), as this is considered to be an optimal compromise between the Kabat and Chothia definitions. Reference is again made to the website http://www.bioinf.org.uk/abs/).

Accordingly, in the present specification and claims, all CDRs or a VHH, Nanobody or ConfoBody are defined according to the Abm convention, unless explicitly stated otherwise herein.

It should be noted that the immunoglobulin single variable domains as binding agent in their broadest sense are not limited to a specific biological source or to a specific method of preparation. The term “immunoglobulin single variable domain” or “ISVD” encompasses variable domains of different origin, comprising mouse, rat, rabbit, donkey, human, shark, camelid variable domains. According to specific embodiments, the immunoglobulin single variable domains are derived from shark antibodies (the so-called immunoglobulin new antigen receptors or IgNARs), more specifically from naturally occurring heavy chain shark antibodies, devoid of light chains, and are known as VNAR domain sequences. Preferably, the immunoglobulin single variable domains are derived from camelid antibodies. More preferably, the immunoglobulin single variable domains are derived from naturally occurring heavy chain camelid antibodies, devoid of light chains, and are known as VHH domain sequences or Nanobodies.

According to a particularly preferred embodiment, the binding agent of the invention is an immunoglobulin single variable domain that is a Nanobody (as defined further herein, and including but not limited to a VHH). The term “Nanobody” (Nb), as used herein, is a single domain antigen binding fragment. It particularly refers to a single variable domain derived from naturally occurring heavy chain antibodies and is known to the person skilled in the art. Nanobodies are usually derived from heavy chain only antibodies (devoid of light chains) seen in camelids (Hamers-Casterman et al. 1993; Desmyter et al. 1996) and consequently are often referred to as VHH antibody or VHH sequence. Camelids comprise old world camelids (Camelus bactrianus and Camelus dromedarius) and new world camelids (for example Lama paccos, Lama glama, Lama guanicoe and Lama vicugna). Nanobody® and Nanobodies® are registered trademarks of Ablynx NV (Belgium). For a further description of VHH's or Nanobodies, reference is made to the book “Single domain antibodies”, Methods in Molecular Biology, Eds. Saerens and Muyldermans, 2012, Vol 911, in particular to the Chapter by Vincke and Muyldermans (2012), as well as to a non-limiting list of patent applications, which are mentioned as general background art, and include: WO 94/04678, WO 95/04079, WO 96/34103 of the Vrije Universiteit Brussel; WO 94/25591, WO 99/37681, WO 00/40968, WO 00/43507, WO 00/65057, WO 01/40310, WO 01/44301, EP 1 134 231 and WO 02/48193 of Unilever; WO 97/49805, WO 01/21817, WO 03/035694, WO 03/054016 and WO 03/055527 of the Vlaams Instituut voor Biotechnologie (VIB); WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As will be known by the person skilled in the art, the Nanobodies are particularly characterized by the presence of one or more Camelidae “hallmark residues” in one or more of the framework sequences (according to Kabat numbering), as described for example in WO 08/020079, on page 75, Table A-3, incorporated herein by reference). It should be noted that the Nanobodies, of the invention in their broadest sense are not limited to a specific biological source or to a specific method of preparation. For example, Nanobodies, can generally be obtained: (i) by isolating the VHH domain of a naturally occurring heavy chain antibody; (ii) by expression of a nucleotide sequence encoding a naturally occurring VHH domain; (iii) by “humanization” of a naturally occurring VHH domain or by expression of a nucleic acid encoding a such humanized VHH domain; (iv) by “camelization” of a naturally occurring VH domain from any animal species, and in particular from a mammalian species, such as from a human being, or by expression of a nucleic acid encoding such a camelized VH domain; (v) by “camelisation” of a “domain antibody” or “Dab” as described in the art, or by expression of a nucleic acid encoding such a camelized VH domain; (vi) by using synthetic or semi-synthetic techniques for preparing proteins, polypeptides or other amino acid sequences known per se; (vii) by preparing a nucleic acid encoding a Nanobody using techniques for nucleic acid synthesis known per se, followed by expression of the nucleic acid thus obtained; and/or (8) by any combination of one or more of the foregoing. A further description of Nanobodies, including humanization and/or camelization of Nanobodies, can be found e.g. in WO08/101985 and WO08/142164, as well as further herein. A particular class of Nanobodies binding conformational epitopes of native targets is called Xaperones and is particularly envisaged here. Xaperone™ is a trademark of VIB and VUB (Belgium). A Xaperone™ is a camelid single domain antibody that constrains drug targets into a unique, disease relevant druggable conformation.

Within the scope of the present invention, the term “immunoglobulin single variable domain” also encompasses variable domains that are “humanized” or “camelized”, in particular Nanobodies that are “humanized” or “camelized”. For example both “humanization” and “camelization” can be performed by providing a nucleotide sequence that encodes a naturally occurring VHH domain or VH domain, respectively, and then changing, in a manner known per se, one or more codons in said nucleotide sequence in such a way that the new nucleotide sequence encodes a “humanized” or “camelized” immunoglobulin single variable domains of the invention, respectively. This nucleic acid can then be expressed in a manner known per se, so as to provide the desired immunoglobulin single variable domains of the invention. Alternatively, based on the amino acid sequence of a naturally occurring VHH domain or VH domain, respectively, the amino acid sequence of the desired humanized or camelized immunoglobulin single variable domains of the invention, respectively, can be designed and then synthesized de novo using techniques for peptide synthesis known per se. Also, based on the amino acid sequence or nucleotide sequence of a naturally occurring VHH domain or VH domain, respectively, a nucleotide sequence encoding the desired humanized or camelized immunoglobulin single variable domains of the invention, respectively, can be designed and then synthesized de novo using techniques for nucleic acid synthesis known per se, after which the nucleic acid thus obtained can be expressed in a manner known per se, so as to provide the desired immunoglobulin single variable domains of the invention. Other suitable methods and techniques for obtaining the immunoglobulin single variable domains of the invention and/or nucleic acids encoding the same, starting from naturally occurring VH sequences or preferably VHH sequences, will be clear from the skilled person, and may for example comprise combining one or more parts of one or more naturally occurring VH sequences (such as one or more FR sequences and/or CDR sequences), one or more parts of one or more naturally occurring VHH sequences (such as one or more FR sequences or CDR sequences), and/or one or more synthetic or semi-synthetic sequences, in a suitable manner, so as to provide a Nanobody of the invention or a nucleotide sequence or nucleic acid encoding the same.

According to a particular embodiment of the present invention, the binding agent that is capable of stabilizing the receptor may bind at the orthosteric site or an allosteric site. In other specific embodiments, the binding agent that is capable of stabilizing the receptor may be an active conformation-selective binding agent, or an inactive conformation-selective binding agent, either by binding at the orthosteric site or at an allosteric site. Generally, a conformation-selective binding agent that stabilizes an active conformation of a receptor will increase or enhance the affinity of the receptor for an active conformation-selective ligand, such as an agonist, more specifically a full agonist, a partial agonist or a biased agonist, as compared to the receptor in the absence of the binding agent (or in the presence of a mock binding agent—also referred to as control binding agent or irrelevant binding agent—that is not directed against and/or does not specifically bind to the receptor). Also, a binding agent that stabilizes an active conformation of a receptor will decrease the affinity of the receptor for an inactive conformation-selective ligand, such as an inverse agonist, as compared to the receptor in the absence of the binding agent (or in the presence of a mock binding agent). In contrast, a binding agent that stabilizes an inactive conformation of a receptor will enhance the affinity of the receptor for an inverse agonist and will decrease the affinity of the receptor for an agonist, particularly for a full agonist, a partial agonist or a biased agonist, as compared to the receptor in the absence of the binding agent (or in the presence of a mock binding agent). An increase or decrease in affinity for a ligand may be directly measured by and/or calculated from a decrease or increase, respectively in EC50, IC50, Kd, K, or any other measure of affinity or potency known to one of skill in the art. It is particularly preferred that the binding agent that stabilizes a particular conformation of a receptor is capable of increasing or decreasing the affinity for a conformation-selective ligand at least 2 fold, at least 5 fold, at least 10 fold, at least 50 fold, and more preferably at least 100 fold, even more preferably at least 1000 fold or more, upon binding to the receptor. It will be appreciated that affinity measurements for conformation-selective ligands that trigger/inhibit particular signaling pathways may be carried out with any type of ligand, including natural ligands, small molecules, as well as biologicals; with orthosteric ligands as well as allosteric modulators; with single compounds as well as compound libraries; with lead compounds or fragments; etc.

The term “affinity”, as used herein, refers to the degree to which a ligand (as defined further herein) binds to a target protein (such as a GPCR) so as to shift the equilibrium of target protein and ligand toward the presence of a complex formed by their binding. Thus, for example, where a GPCR and a ligand are combined in relatively equal concentration, a ligand of high affinity will bind to the available antigen on the GPCR so as to shift the equilibrium toward high concentration of the resulting complex. The dissociation constant is commonly used to describe the affinity between a ligand and a target protein. Typically, the dissociation constant is lower than 10-5 M. Preferably, the dissociation constant is lower than 106 M, more preferably, lower than 10-7 M. Most preferably, the dissociation constant is lower than 10-8 M. Other ways of describing the affinity between a ligand (including small molecule ligands) and its target protein are the association constant (Ka), the inhibition constant (Ki) (also referred to as the inhibitory constant), or indirectly by evaluating the potency of ligands by measuring the half maximal inhibitory concentration (IC50) or half maximal effective concentration (EC50). Within the scope of the present invention, the ligand may be a binding agent, preferably an immunoglobulin, such as an antibody, or an immunoglobulin fragment, such as a VHH or Nanobody, that binds a conformational epitope on a GPCR. It will be appreciated that within the scope of the present invention, the term “affinity” is used in the context of a binding agent, in particular an immunoglobulin or an immunoglobulin fragment, such as a VHH or Nanobody, that binds a conformational epitope of a target GPCR as well as in the context of a test compound (as defined further herein) that binds to a target GPCR, more particularly to an orthosteric or allosteric site of a target GPCR.

The term “specificity”, as used herein, refers to the ability of a protein or other binding agent, in particular an immunoglobulin or an immunoglobulin fragment, such as a VHH or Nanobody, to bind preferentially to one antigen (such as a GPCR), versus a different antigen (such as a different GPCR), and does not necessarily imply high affinity.

The terms “specifically bind” and “specific binding”, as used herein, generally refers to the ability of a binding agent, in particular an immunoglobulin, such as an antibody, or an immunoglobulin fragment, such as a VHH or Nanobody, to preferentially bind to a particular antigen that is present in a homogeneous mixture of different antigens. In certain embodiments, a specific binding interaction will discriminate between desirable and undesirable antigens in a sample, in some embodiments more than about 10 to 100-fold or more (e.g., more than about 1000- or 10,000-fold). Within the context of the spectrum of conformational states of GPCRs, the terms particularly refer to the ability of a binding agent (as defined herein) to preferentially recognize and/or bind to a particular conformational state of a GPCR as compared to another conformational state.

Also, it should be understood that in the present description and appended claims, where a protein, ligand, compound, binding domain, binding unit or other chemical entity is said to “bind” another protein, ligand, compound, binding domain, binding unit or other chemical entity or an epitope or binding site, that such binding is preferably “specific” binding as defined herein. Also, preferably, such binding is “selective binding” as defined herein.

As used herein, the term “conformation-selective binding agent” in the context of the present invention refers to a binding agent that binds to a target protein (such as a GPCR) in a conformation-selective manner. A binding agent that selectively binds to a particular conformation or conformational state of a protein refers to a binding agent that binds with a higher affinity to a protein in a subset of conformations or conformational states than to other conformations or conformational states that the protein may assume. One of skill in the art will recognize that binding agents that selectively bind to a specific conformation or conformational state of a protein will stabilize or retain the protein it this particular conformation or conformational state. For example, an active conformation-selective binding agent will preferentially bind to a GPCR in an active conformational state and will not or to a lesser degree bind to a GPCR in an inactive conformational state, and will thus have a higher affinity for said active conformational state; or vice versa. The terms “specifically bind”, “selectively bind”, “preferentially bind”, and grammatical equivalents thereof, are used interchangeably herein. The terms “conformational specific” or “conformational selective” are also used interchangeably herein (but it should be noted that the term “conformation-inducing” as used herein has a separate meaning that is as further defined herein).

As used herein, the term “stabilizing”, or grammatically equivalent terms, as defined hereinbefore, is meant an increased stability of a protein (as described herein) or receptor (also as described herein) with respect to the structure (e.g. conformational state) and/or particular biological activity (e.g. intracellular signaling activity, ligand binding affinity, . . . ). In relation to increased stability with respect to structure and/or biological activity, this may be readily determined by either a functional assay for activity (e.g. Ca2+ release, cAMP generation or transcriptional activity, β-arrestin recruitment, . . . ) or ligand binding or by means of physical methods such as X-ray crystallography, NMR, or spin labeling, among other methods. The term “stabilize” also includes increased thermostability of the receptor under non-physiological conditions induced by denaturants or denaturing conditions. The term “thermostabilize”, “thermostabilizing”, “increasing the thermostability of”, as used herein, refers to the functional rather than to the thermodynamic properties of a receptor and to the protein's resistance to irreversible denaturation induced by thermal and/or chemical approaches including but not limited to heating, cooling, freezing, chemical denaturants, pH, detergents, salts, additives, proteases or temperature. Irreversible denaturation leads to the irreversible unfolding of the functional conformations of the protein, loss of biological activity and aggregation of the denaturated protein. In relation to an increased stability to heat, this can be readily determined by measuring ligand binding or by using spectroscopic methods such as fluorescence, CD or light scattering that are sensitive to unfolding at increasing temperatures. It is preferred that the binding agent is capable of increasing the stability as measured by an increase in the thermal stability of a protein or receptor in a functional conformational state with at least 2° C., at least 5° C., at least 8° C., and more preferably at least 10° C. or 15° C. or 20° C. In relation to an increased stability to a detergent or to a chaotrope, typically the protein or receptor is incubated for a defined time in the presence of a test detergent or a test chaotropic agent and the stability is determined using, for example, ligand binding or a spectroscopic method, optionally at increasing temperatures as discussed above. Otherwise, the binding agent is capable of increasing the stability to extreme pH of a functional conformational state of a protein or receptor. In relation to an extreme of pH, a typical test pH would be chosen for example in the range 6 to 8, the range 5.5 to 8.5, the range 5 to 9, the range 4.5 to 9.5, more specifically in the range 4.5 to 5.5 (low pH) or in the range 8.5 to 9.5 (high pH). The term “(thermo) stabilize”, “(thermo) stabilizing”, “increasing the (thermo) stability of”, as used herein, applies to protein or receptors embedded in lipid particles or lipid layers (for example, lipid monolayers, lipid bilayers, and the like) and to proteins or receptors that have been solubilized in detergent.

In addition to the foregoing, with respect to a functional conformational state of a GPCR, the term “stabilizing” or “stabilized” refers to the retaining or holding of a GPCR protein in a subset of the possible conformations that it could otherwise assume, due to the effects of the interaction of the GPCR with the binding agent according to the invention. Within this context, a binding agent that selectively binds to a specific conformation or conformational state of a protein refers to a binding agent that binds with a higher affinity to a protein in a subset of conformations or conformational states than to other conformations or conformational states that the protein may assume. One of skill in the art will recognize that binding agents that specifically or selectively bind to a specific conformation or conformational state of a protein will stabilize this specific conformation or conformational state, and its related activity. More details are provided further herein.

The term “compound” or “test compound” or “candidate compound” or “drug candidate compound” as used herein describes any molecule, either naturally occurring or synthetic that is tested in an assay, such as a screening assay or drug discovery assay. As such, these compounds comprise organic or inorganic compounds. The compounds include polynucleotides, lipids or hormone analogs that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies, antibody fragments or antibody conjugates. Test compounds can also be protein scaffolds. For high-throughput purposes, test compound libraries may be used, such as combinatorial or randomized libraries that provide a sufficient range of diversity. Examples include, but are not limited to, natural compound libraries, allosteric compound libraries, peptide libraries, antibody fragment libraries, synthetic compound libraries, fragment-based libraries, phage-display libraries, and the like. A more detailed description can be found further in the specification.

As used herein, the term “ligand” means a molecule that specifically binds to a protein referred to herein, such as to a GPCR. A ligand may be, without the purpose of being limitative, a polypeptide, a lipid, a small molecule, an antibody, an antibody fragment, a nucleic acid, a carbohydrate. A ligand may be synthetic or naturally occurring. A ligand also includes a “native ligand” which is a ligand that is an endogenous, natural ligand for a native GPCR. Within the context of the present invention, when a protein is a transmembrane protein such as a GPCR, a ligand may bind to said protein either on a ligand binding site that is exposed to the intracellular environment when the protein is in its native cellular environment (i.e. the ligand may be an “intracellular ligand”), or the ligand may bind to said protein on a ligand binding site that is exposed to the environment outside of the cell when the protein is in its native cellular environment (i.e. the ligand may be an “extracellular ligand”). Extracellular ligands are often classified based on the way in which they act to modulate (as defined herein) the GPCR, for example as an agonist, as a partial agonist, as an inverse agonist, as an antagonist or as an allosteric modulator. An extracellular ligand may bind at either the orthosteric site or at an allosteric site.

As further described herein, an intracellular ligand (such as a binding domain or binding unit that is used in the present invention) may be a “conformation-inducing” (as defined herein) ligand, meaning that said ligand is capable of stabilizing and/or inducing a functional and/or active conformational state of the chimeric GPCR upon binding to the chimeric GPCR (i.e. to the intracellular binding site on the chimeric GPCR). As also further described herein, such a conformation-inducing ligand may also be capable of inducing the formation of and/or stabilizing a complex formed by the GPCR (which in said complex is then preferably in a functional, active and/or druggable state), the conformation-inducing intracellular ligand and an extracellular ligand (in particular when the extracellular ligand can act as an agonist on the GPCR.

In particular embodiments, an (extracellular or intracellular) ligand may be a “conformation-selective ligand” or “conformation-specific ligand”, meaning that such a ligand binds the protein or GPCR in a conformation-selective manner. As further described herein, a conformation-selective ligand binds with a higher affinity to a particular conformation of the protein than to other conformations the protein may adopt. For the purpose of illustration, an extracellular ligand that acts as an agonist is an example of an active conformation-selective ligand, whereas an extracellular ligand that acts as an inverse agonist is an example of an inactive conformation-selective ligand. For the sake of clarity, a neutral antagonist is not considered as a conformation-selective ligand, since a neutral antagonist does not distinguish between the different conformations of a GPCR.

An “orthosteric ligand”, as used herein, refers to a ligand (both natural and synthetic), that binds to the active site of a GPCR, and are further classified according to their efficacy or in other words to the effect they have on signaling through a specific pathway. As used herein, an “agonist” refers to a ligand that, by binding a receptor protein (such as a GPCR), increases the receptor's signaling activity. Full agonists are capable of maximal protein stimulation; partial agonists are unable to elicit full activity even at saturating concentrations. Partial agonists can also function as “blockers” by preventing the binding of more robust agonists. An “antagonist”, also referred to as a “neutral antagonist”, refers to a ligand that binds a receptor without stimulating any activity. An “antagonist” is also known as a “blocker” because of its ability to prevent binding of other ligands and, therefore, block agonist-induced activity. Further, an “inverse agonist” refers to an antagonist that, in addition to blocking agonist effects, reduces a receptor's basal or constitutive activity below that of the unliganded protein.

Ligands as used herein may also be “biased ligands” with the ability to selectively stimulate a subset of a receptor's signaling activities, for example in the case of GPCRs the selective activation of G-protein or β-arrestin function. Such ligands are known as “biased ligands”, “biased agonists” or “functionally selective agonists”. More particularly, ligand bias can be an imperfect bias characterized by a ligand stimulation of multiple receptor activities with different relative efficacies for different signals (non-absolute selectivity) or can be a perfect bias characterized by a ligand stimulation of one receptor protein activity without any stimulation of another known receptor protein activity.

Another kind of ligands is known as allosteric regulators. “Allosteric regulators” or otherwise “allosteric modulators”, “allosteric ligands” or “effector molecules”, as used herein, refer to ligands that bind at an allosteric site (that is, a regulatory site physically distinct from the protein's active site) of a GPCR. In contrast to orthosteric ligands, allosteric modulators are non-competitive because they bind receptor proteins at a different site and modify their function even if the endogenous ligand also is binding. Allosteric regulators that enhance the protein's activity are referred to herein as “allosteric activators” or “positive allosteric modulators” (PAMs), whereas those that decrease the protein's activity are referred to herein as “allosteric inhibitors” or otherwise “negative allosteric modulators” (NAMs).

As used herein, the terms “determining”, “measuring”, “assessing”, “assaying” are used interchangeably and include both quantitative and qualitative determinations.

The term “antibody” is intended to mean an immunoglobulin or any fragment thereof that is capable of antigen binding. The term “antibody” also refers to single chain antibodies and antibodies with only one binding domain.

As used herein, the terms “complementarity determining region” or “CDR” within the context of antibodies refer to variable regions of either H (heavy) or L (light) chains (also abbreviated as VH and VL, respectively) and contains the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains. Immunoglobulin single variable domains, in particular Nanobodies, generally comprise a single amino acid chain that can be considered to comprise 4 “framework sequences or regions” or FRs and 3 “complementary determining regions” or CDRs. The nanobodies have 3 CDR regions, each non-contiguous with the others (termed CDR1, CDR2, CDR3). As mentioned herein, for denoting the amino acid positions/residues CDRs in a VHH, Nanobody or ConfoBody, the Kabat numbering system will be followed, and the frameworks and CDRs are defined on the basis of the Abm definitions (unless explicitly stated otherwise).

Generally, for the purposes of the disclosure herein and its appended claims, a compound of the invention will be considered to a “modulator” of a target, or to “modulate” a target (and/or of the signaling, pathway(s), mechanism of action and/or said biological, physiological and/or pharmacological functions in which said target is involved) when the presence of the compound (i.e. in a suitable amount or concentration, such as a biologically active amount or concentration) in a suitable assay or model changes a suitable or intended read-out of said assay or model (i.e. at least one suitable value or parameter that can be determined using said assay or model) by at least 0.1%, such as at least 1%, for example at least 10% and up to 50% or more, compared to the same value or parameter when it is measured using the same assay or model under essentially the same conditions but without the presence of said compound. Again, said modulation may result in an increase or a decrease of said value or parameter (i.e. by the percentages given in the previous sentence). Also, a compound of the invention will preferably be such that it can modulate said target, signaling, pathway(s), mechanism of action and/or said biological, physiological and/or pharmacological functions in a dose-dependent manner, i.e. in or over at least one range of concentrations of the compound used in the assay or model.

There are numerous prior art references that discuss the sequence and structure of GPCRs. Next to the prior art already cited herein, these include Mirzadegan and Benko, Biochemistry. Mar. 18, 2003; 42(10): 2759-2767; Arakawa et al., Biochimica et Biophysica Acta 1808 (2011) 1170-1178; Han et al., FEBS Open Bio 5 (2015) 182-190; Sanchez-Reyes et al., Biophysical Journal 112, 2315-2326, Jun. 6, 2017 2315; and Kochman, Postepy Hig Med Dosw (Online). Oct. 31, 2014; 68:1225-37. Mirzadegan and Benko give the results of multiple sequence analyses based on homology that were performed on 270 GPCRs of family A (of which 153 were orphan GPCRs with unknown ligands). They indicate that the length of GPCRs from family A varies between 290 and 951 amino acid residues, with the majority of receptors having a length around 310-470 residues. They also indicate that the GPCRs are characterized by a set of conserved residues distributed among the seven helical domains, which facilitate multiple alignments between GPCR sequences. These conserved residues (which are also sometimes referred to in the art as “signature residues”) are in helix I (Gly and Asn), helix II (Leu and Asp), helix III (Cys and AspArgTyr), helix IV (Trp and Pro), helix V (Pro and Tyr), helix VI (Phe, Trp, and Pro), and helix VII (Asn, Pro, and Tyr of the NPXXY motif). Sanchez-Reyes et al in Table S1 also list GPCR signature residues and their degree of conservation within Family A, which signature residues are at the following positions:

TABLE A
Signature residues in Family A GPCRs
(according to Sanchez-Reyes et al., 2017):

| Position | Residue |
|---|---|
| 1.49 | Gly (G) |
| 1.50 | Asn (N) |
| 2.46 | Leu (L) |
| 2.47 | Ala (A) |
| 2.50 | Asp (D) |
| 3.25 | Cys (C) |
| 3.39 | Ser (S) |
| 3.43 | Leu (L) |
| 3.49 | Asp (D) |
| 3.50 | Arg (R) |
| 3.51 | Tyr (Y) |
| 4.50 | Trp (W) |
| 5.50 | Pro (P) |
| 5.58 | Tyr (Y) |
| 6.44 | Phe (F) |
| 6.47 | Cys (C) |
| 6.48 | Trp (W) |
| 6.50 | Pro (P) |
| 7.49 | Asn (N) |
| 7.50 | Pro (P) |
| 7.53 | Tyr (Y) |

Note:
Reyes et al use the “corrected structure-based Ballesteros-Weinstein numbering system” for describing the relative position of amino acids within a TM sequence in the family A GPCRs, indicating as well the residue identity with the highest conservation in the family A receptors. This numbering system is also used in the present description and claims, unless indicated otherwise. Reference is made to Ballesteros and Weinstein, Methods Neurosci. 25: 366-428; and to Isberg, cited herein.

Most of these signature residues are for example also indicated in red in FIG. 1 of Arakawa et al., which schematically shows their position within the overall structure of the beta-adrenergic receptor.

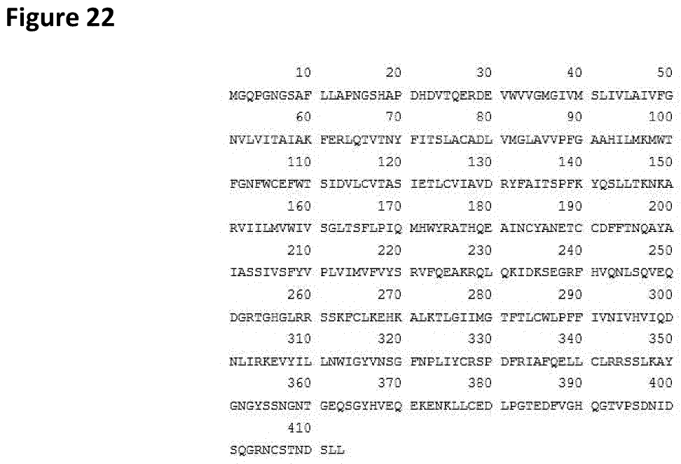

Alternatively to Table A, the position of the various signature residues can be understood by their relative relation to particular residues in a chosen canonical GPCR. Thus, when examining a position in a particular GPCR, reference can be made to the relative location in the overall structure of the canonical GPCR in order to determine if the residue being considered is present in that location on the particular GPCR. If the human β₂AR (UniProt P07550 (ADRB2_HUMAN), see SEQ ID NO: 17 and FIG. 22) is taken as the base GPCR then—the signature residues can be described as:
a Gly at a position relative to amino acid 50 of ADRB2_HUMAN;
a Ans at a position relative to amino acid 51 of ADRB2_HUMAN;
a Leu at a position relative to amino acid 75 of ADRB2_HUMAN;
a Ala at a position relative to amino acid 76 of ADRB2_HUMAN;
a Asp at a position relative to amino acid 79 of ADRB2_HUMAN;
a Cys at a position relative to amino acid 106 of ADRB2_HUMAN;
a Ser at a position relative to amino acid 120 of ADRB2_HUMAN;
a Leu at a position relative to amino acid 124 of ADRB2_HUMAN;
a Asp at a position relative to amino acid 130 of ADRB2_HUMAN;
a Arg at a position relative to amino acid 131 of ADRB2_HUMAN;
a Tyr at a position relative to amino acid 132 of ADRB2_HUMAN;
a Trp at a position relative to amino acid 158 of ADRB2_HUMAN;
a Pro at a position relative to amino acid 211 of ADRB2_HUMAN;
a Tyr at a position relative to amino acid 219 of ADRB2_HUMAN;
a Phe at a position relative to amino acid 282 of ADRB2_HUMAN;
a Cys at a position relative to amino acid 285 of ADRB2_HUMAN;
a Trp at a position relative to amino acid 286 of ADRB2_HUMAN;
a Pro at a position relative to amino acid 288 of ADRB2_HUMAN;
a Asn at a position relative to amino acid 322 of ADRB2_HUMAN;
a Pro at a position relative to amino acid 323 of ADRB2_HUMAN; and
a Tyr at a position relative to amino acid 326 of ADRB2_HUMAN.

As described herein, the present invention aims to provide a methodology for providing assay and screening techniques for a desired GPCR that do not require conformation-specific VHHs to be raised or generated against (the intracellular part(s) of) said specific GPCRs, and thus to avoid any issues or limitations of prior art methodology that may be associated with the need to provide the desired GPCR in an isolated and suitably purified form and in a desired conformation for screening and selection purposes and, when naïve libraries are to be used, for immunization and display purposes.

The invention generally achieves this objective by providing the chimeric proteins described herein and by using said chimeric proteins together with binding domains or binding units that are specific for (the binding site formed by) the intracellular loops of the chimeric protein (as further described herein). Such chimeric GPCRs, in their various aspects and embodiments as described herein, form a first aspect of the invention, and said chimeric proteins (which are also referred to herein as “chimeric proteins of the invention” or “chimeric GPCRs of the invention”, which terms are used interchangeably herein), such binding domains or binding units that can bind to the chimeric GPCR, and their use in the invention are as further described herein.

Generally, as further described herein, the chimeric proteins of the invention have at least one or more parts of their amino acid sequence that are derived from a first GPCR and at least one or more other parts of their sequence that are derived from a second GPCR (different from the first). In particular, the chimeric proteins of the invention have (at least) extracellular loops that are derived from a first GPCR and intracellular loops that are derived from a second GPCR (different from the first).

Preferably, both said first and said second GPCR are naturally occurring GPCRs. Also, when the invention is to be used to discover or develop pharmaceuticals, at least the first GPCR and preferably also the second GPCR are GPCRs that naturally occur in the body of a human being (i.e. on the surface of at least one cell that is present in the body of a human being), and in particular in the body of a subject that is to be treated with a compound, ligand or other therapeutic entity that has been discovered and/or developed using the chimeric proteins and methods described herein (i.e. on the surface of at least one cell that is present in the body of said subject), for example for the purposes of therapy or prophylaxis (as further described herein, one preferred use of the invention is to generate compounds that can modulate—as defined herein—the “first” GPCR from which the ECLs have been derived).

Although usually and preferably, the chimeric proteins of the invention will essentially consist of (and/or only be comprised of) stretches of amino acid residues that are derived either from the first or the second GPCR, it is not excluded from the scope of the invention in its broadest sense that the chimeric proteins of the invention will also suitably comprise one or more stretches of amino acid residues that are derived from one or more other GPCRs, one or more stretches of amino acid residues that are derived from other proteins (although this will usually be less preferred) and/or one or more stretches of amino acid residues that are synthetic or semi-synthetic, for example obtained by introducing one or more mutations (as defined herein) into a stretch of amino acid residues that has been obtained from the first or second (or another) GPCR.

The chimeric proteins of the invention will generally and preferably comprise an N-terminal sequence, a C-terminal sequence, 7 transmembrane domains (TMs), 3 extracellular loops (ECs or ECLs) and 3 intracellular loops (ICs or ICLs). More preferably, in a chimeric protein of the invention, these parts of the overall sequence are arranged into the structure that is most common for naturally occurring GPCRs, i.e. as follows (from the N-terminal end to its C-terminal end):

[N-terminal sequence]-[TM1]-[IC1]-[TM2]-[EC1]-[TM3]-[IC2]-[TM4]-[EC2]-[TM5]-[IC3]-[TM6]-[EC3]-[TM7]-[C-terminal sequence].

In particular, in a chimeric protein of the invention, at least one (such as at least two) of the extracellular loops are derived from a first GPCR and at least one (such as at least two) of the intracellular loops are be derived from a second GPCR (different from the first).

Most preferably, in a chimeric protein of the invention, all three (or essentially all three) of the extracellular loops are derived from a first GPCR and all three (or essentially all three) of the intracellular loops are derived from a second GPCR (different from the first). This should be understood to mean that the extracellular loops preferably have no more than 2, more preferably no more than 1, and most preferred no amino acid differences (as defined herein) with the extracellular loops of the (first) GPCR from which said extracellular loops have been derived, and that the intracellular loops preferably have no more than 2, more preferably no more than 1, and most preferred no amino acid differences (as defined herein) with the intracellular loops of the (second) GPCR from which said intracellular loops have been derived.

The TMs that are present in the chimeric protein of the invention are preferably all essentially derived from the same GPCR. More preferably, the TMs that are present in the chimeric protein of the invention are such that they, together with the extracellular loops, form a functional ligand binding site, and in particular a ligand binding site that closely resembles and/or mimics the binding site for extracellular ligands of the (first) GPCR from which the extracellular loops have been derived. Usually in the invention, and preferably, this means that the TMs are essentially the same as, and/or are essentially derived from, the first GPCR (except that, as further described herein and dependent how the ICLs are provided/inserted into the chimeric GPCR, they may contain some amino acid residues which are the same as and/or derived from the second GPCR in positions of the TM7 that are adjacent to the ICLs). In particular, the parts of the sequence of the chimeric GPCR of the invention that are derived from the first GPCR may be such that they form a functional binding site for extracellular ligands which is essentially the same as and/or closely mimics the extracellular binding site of the first GPCR from which said parts of the chimeric sequence have been derived.

Preferably, each TM in the chimeric GPCR has no more than 4, more preferably no more than 3, such as no more than 2, and in particular no more than 1 and most preferably no amino acid differences with the amino acid sequence of the corresponding TM that is present in the naturally occurring GPCR from which said TM has been derived (not taking into account any amino acid residues that are the same as those present in and/or that are derived from the second GPCR in positions next to the ICLs).

Also, when taking into account any amino acid residues that are the same as those present in and/or that are derived from the second GPCR in positions next to the ICLs, each such TM preferably has at least 80%, more preferably at least 85%, such as at least 90%, for example more than 95% and up to and including 100%, sequence identity with the amino acid sequence of the corresponding TM from the naturally occurring GPCR from which said TM has been derived (again also depending on how many any amino acid residues in each TM are the same as and/or derived from the second GPCR). When any amino acid residues that are the same as those present in and/or that are derived from the second GPCR in positions next to the ICLs are not taken into account, each such TM preferably has at least 90%, more preferably at least 95%, such as at least 98%, and up to and including 100%, sequence identity with the amino acid sequence of the corresponding TM from the naturally occurring GPCR from which said TM has been derived.

The N-terminal sequence of the chimeric protein of the invention will usually be derived from the same GPCR as the first of the TMs (and as described herein, in the practice of the invention, this will usually and preferably be first GPCR). The C-terminal sequence will usually also be derived from the same GPCR as TM7 (and as described herein, in the practice of the invention, this will again usually and preferably be first GPCR). However, it is also possible that the C-terminal part is derived from the second GPCR, and it may be that using the C-terminal part of the second GPCR may improve expression levels and/or other properties (i.e. compared to the same chimeric GPCR but with the C-terminal sequence from the first GPCR).

In one aspect of the invention, the amino acid sequences that are derived from the first GPCR and the amino acid sequences that are derived from the second GPCR are all derived from GPCRs that belong to the same class or family of GPCRs (in other words, in the invention, the first and second GPCR preferably belong to the same class of family of GPCRs). Thus, when the standard classification from the IUPHAR database (as of January 2019) is used, the first and second GPCR both preferably belong to Class A (rhodopsin-like), to Class B (secretin receptor family), to Class C (metabotropic glutamate) or to Class F (frizzled/smoothened) (as the present invention is mainly directed towards applications in vertebrate animals and in particular in human beings, GPCR sequences from Classes D and E will usually not find any utility in the present invention). When the “GRAFS” classification for GPCRs from vertebrates is used, the first and second GPCR both preferably belong to the Glutamate family, the Rhodopsin family, the Adhesion family, the Frizzled family or the Secretin family. However, surprisingly, as will be seen from the experimental data shown herein, it is also possible in the invention to provide and/or use a chimeric GPCR of the invention in which the ECs and TMs have essentially been derived from a GPCR from one class or family and the ICLs have essentially been derived from another class or family.

Preferably, the ICLs are derived from a GPCR belonging to Class A (rhodopsin-like) (classification according to the IUPHAR database as of January 2019). Also, the ECLs, the TMs and the C-terminal and N-terminal sequence are also preferably derived from a GPCR belonging to Class A (rhodopsin-like). Thus, more generally in the invention, the first GPCR is preferably a GPCR belonging to Class A and the second GPCR is preferably a GPCR belonging to Class A. Reference is also made to the experimental section, in which some particularly preferred combinations of ICLs and VHHs specific for said ICls are used.

For example and without limitation, in one aspect, the ICLs may be obtained from the beta-2-adrenergic receptor, and the binding domain may be an ISVD binding to the ICLs of the beta-2-adrenergic receptor, such as one of the ISVDs described in the International Application 2012/007593, which also gives the sequences and CDRs of particular VHHs that are conformation-inducing (as defined herein) with respect to the beta-adrenergic receptor, such as for example CA2780 (SEQ ID NO: 4 in WO2012/007593 and SEQ ID NO:20 herein), which is also referred to as “Nb80” and which is also used in the Experimental Part below).

In another non-limiting aspect, the ICLs may be obtained from an opioid receptor (and in particular the Mu-opioid receptor), and the binding domain may be an ISVD binding to the ICLs of an opioid receptor (and in particular the Mu-opioid receptor), such as one of the ISVDs described in the International Application 2015/121092, which also gives the sequences and CDRs of particular VHHs that are conformation-inducing (as defined herein) with respect to the Mu-opioid receptor, such as for example X8633 (SEQ ID NO: 19 in WO2014/118297 and SEQ ID NO: 21 herein) which is also used in the Experimental Part below.

The ICLs used are preferably further such (and are incorporated into the chimeric GPCR of the invention such) that they form a functional binding site, in particular a functional binding site for the binding domain or binding unit that is used in the methods of the invention (which binding domain or binding unit, as further described herein, is most preferably a conformation-inducing binding domain or binding unit and in particular a conformation-inducing ISVD such as a ConfoBody). More in particular, the ICLs used are such that, together with the rest of the chimeric GPCR, they form (part of) a conformational epitope, i.e. an epitope or binding site that changes it “shape” (e.g. its geometry and/or spatial arrangement) depending on the conformational state of the chimeric GPCR, for example when the chimeric GCPR undergoes a conformational change, such as a conformational change from an inactive or less active state into an active, more active and/or functional state and/or a conformational change that occurs when a first ligand binds to the extracellular binding site on the chimeric GPCR (essentially similar to the conformational changes that a naturally occurring GPCRs can undergo, for example when it is bound by an agonist).

Also, the ICLs used are preferably further such (and are incorporated into the chimeric GPCR of the invention such) that they form a functional (intracellular) binding site that mimics the corresponding binding site on the naturally occurring GPCR from which said ICLs have been derived. In particular, the ICLs used may be such that they mimic the G-protein binding site of the naturally occurring GPCR from which the ICLs have been derived. Thus, in one specific aspect, the chimeric protein of the invention is such that its ICLs form (or form part of) a functional binding site for a G-protein or G-protein complex, as further described herein. Often, in the invention, the chimeric protein of the invention will be such that its ICLs form (or form part of) a functional binding site that is both a functional binding site for a G-protein or G-protein complex as well as being a functional binding site for a specific VHH raised against said ICLs.

Thus, generally, a chimeric GPCR of the invention will comprise at least two distinct ligand binding sites, i.e. at least:
a first ligand binding site that most preferably closely mimics or essentially corresponds to the extracellular ligand binding site on the GPCR from which the extracellular loops (and preferably also the TMs) have been derived. As is known for GPCRs generally, said ligand binding site may be formed by one or more of the ECLs and/or one or more of the TMs, but will not comprise the ICLs. Said ligand binding site on the chimeric GPCR of the invention will also be generally referred to herein as the “extracellular binding site”. As further described herein, and as will also be clear to the skilled person, usually (and preferably) said extracellular binding site on the chimeric GPCR of the invention will correspond to the orthosteric binding site of the first GPCR from which the ECLs (and preferably also the TMs) have been derived (although as mentioned herein, the term “extracellular binding site” in its broadest sense as used herein also includes suitable allosteric binding sites); and
a second ligand binding site that comprises at least one (and preferably at least two such as all three) of the ICLs. As described herein, said ligand binding site should be such that it can be bound by the protein binding domain (in particular, the ISVD) used in the present invention. Also, preferably, said ligand binding site comprises and/or mimics a G-protein binding site. This ligand binding site on the chimeric GPCR of the invention will also be generally referred to herein as the “intracellular binding site”. However, it should be noted that, as is also known per se for naturally occurring GPCRs (reference is for example made to Eglen and Reisine, cited herein), a chimeric GPCR of the invention may, in addition to the binding site that corresponds to the orthosteric binding site of the first GPCR, also contain one or more allosteric binding sites, depending on the ECLs and TMs that are present in the chimeric GPCR of the invention. Preferably, when the ECLs and TMs are both derived from the first GPCR, the chimeric GPCR of the invention also contains essentially the same allosteric binding sites as the first GPCR. Thus, it is envisaged that the present invention can also be used to identify, generate, screen for, test and/or develop compounds and ligands that are directed against an allosteric binding site. As discussed by Eglen and Reisine (cited herein) such allosteric binders may be valuable alternatives for ligands and compounds that are directed against the orthosteric binding site, in particular for therapeutic uses.

Accordingly, it should be understood that, although the term “extracellular binding site” as used in the present description and claims preferably refers to an orthosteric binding site (i.e. the orthosteric binding site of the GPCR from which the ECLs have been derived), the term “extracellular binding site” in its broadest sense also includes allosteric binding sites, and in particular allosteric binding sites that, in the GPCR from which the ECLs have been derived when said GPCR is in its native cellular environment extend out (as defined herein) into the extracellular environment.

With respect to the terms “extracellular binding site” and the “intracellular binding site”, respectively, it should also be understood that the use of these terms does not mean or imply that a chimeric GPCR of the invention needs to be present in a cellular environment. Instead, these binding sites are referred to by these terms because the extracellular binding site on the chimeric GPCR will generally be provided so as essentially correspond to and/or closely mimic the extracellular binding site(s) (i.e. at least the orthosteric site and optionally also one or more allosteric sites, if present) oihu[ of the naturally occurring GPCRs from which the ECLs (and usually also the TMs) have been derived and because the intracellular binding site on the chimeric GPCR will generally be provided so as essentially correspond to and/or closely mimic the intracellular binding site of the naturally occurring GPCRs from which the ICLs have been derived, respectively.

Thus, in one aspect, the invention relates to a chimeric GPCR as further described herein which comprises an extracellular binding site which is as further described herein and an intracellular binding site which is as further described herein.

The invention further relates to a chimeric GPCR which comprises an extracellular binding site that (essentially) is derived from a first GPCR and an intracellular binding site that (essentially) is derived from a second GPCR (different from the first). The invention also relates to a composition that comprises such a chimeric GPCR, which composition can be as further described herein. The invention further relates to a composition that comprises such a chimeric GPCR and that further comprises a binding domain or binding unit that can specifically bind to intracellular binding site on said chimeric GPCR. Again, such a composition can be as further described herein, and the binding domain or binding unit that is present in said composition is preferably a conformation-inducing (as defined herein) binding domain or binding unit and more preferably a conformation-inducing ISVD (such as a ConfoBody).

The invention further relates to a chimeric GPCR that comprises ECLs and TMs which are derived from a first GPCR, which ECLs and TMs are such that the chimeric GPCR comprises a (functional) extracellular binding site that (essentially) corresponds to (and/or closely mimics) the extracellular binding site of said first GPCR, that comprises ICLs which are derived from a second GPCR (different from the first), which ICLs are such that they form (part of) a functional intracellular binding site. The invention further relates to a composition that comprises such a chimeric GPCR and that further comprises a binding domain or binding unit that can specifically bind to said intracellular binding site on said chimeric GPCR. Again, such a composition can be as further described herein, and the binding domain or binding unit that is present in said composition is preferably a conformation-inducing (as defined herein) binding domain or binding unit and more preferably a conformation-inducing ISVD (such as a ConfoBody).

In another aspect, the invention relates to a composition which at least comprises:
a) a chimeric protein comprising an N-terminal sequence, a C-terminal sequence, 7 transmembrane domains (TMs), 3 extracellular loops and 3 intracellular loops, in which:
   the extracellular loops that are present in the chimeric protein are (essentially) derived from a first GPCR; and
   the intracellular loops that are present in the chimeric protein are (essentially) derived from a second GPCR;
      and
b) a binding domain or binding unit that can specifically bind to (the binding site formed by) the intracellular loops that are present in said chimeric protein, which binding domain or binding unit is preferably a conformation-inducing (as defined herein) binding domain or binding unit and more preferably a conformation-inducing ISVD (such as a ConfoBody).

In such a composition, the chimeric protein and the binding domain or binding unit are preferably as further described herein.

In one aspect, the binding domain or binding unit may be fused to the chimeric protein, essentially as described in the International application WO 2014/118297, which describes fusions of GPCRs and ConfoBodies and uses thereof. Thus, in a further aspect, the invention relates to a fusion protein that comprises a chimeric GPCR of the invention that is fused, either directly or via a suitable linker or spacer, and preferably at its C-terminal end, to a binding domain or binding unit as further described herein (which binding domain or binding unit is preferably a conformation-inducing binding domain or binding unit, and preferably a conformation-inducing ISVD).

In another aspect (which is also as described herein), the invention relates to a fusion protein that comprises a chimeric GPCR of the invention that is fused, either directly or via a suitable linker or spacer, to a binding domain or binding unit that is a first binding member of a binding pair that comprises at least a first and a second binding member, which binding pair can generate a detectable signal when said first and second binding member come into contact or in close proximity to each other.

In a more general aspect, the invention relates to fusion proteins that comprise a chimeric GPCR of the invention and at least one further amino acid sequence, protein or peptide (such as at least one binding domain or binding unit).

As also further described herein, a composition comprising a chimeric protein of the invention (and preferably also a binding domain or binding unit as described herein) can be a cell, a cell line or a suitable fractions or preparations that are derived from a cell or cell line such as a membrane fraction, a cell fraction comprising one or more kinds of organelles or a suitable cell lysate (such cells and fractions derived from such cells are also referred to herein as “cellular composition”). Such a composition may also be a liposome, vesicle or other suitable a liposomal composition which may comprise natural or synthetic lipids or a combination thereof, including but not limited to Virus Like Lipoparticles, lipid layers (bilayers and monolayers), lipid vesicles, high-density lipoparticles (e.g. nanodisks), and the like. Usually, the composition will be such that, and the chimeric GPCR will be present in said composition in such a way that, the GPCR can take on the barrel-like tertiary structure that is characteristic of GPCRs. Often, this will mean that the GPCR will be suitably associated with (e.g. suitably anchored in or to) one or more other components of the composition such as a cell wall, cell membrane, a fragment of a cell wall or cell membrane, the wall of a liposome or vesicle, or a lipid bilayer in such a way that, the GPCR can take on the barrel-like tertiary structure that is characteristic of GPCRs. Also, often, the composition will be such that, and the chimeric GPCR will be present in said composition in such a way that, at least at the scale of the size of the GPCR, the extracellular binding site of the GPCR is separated from the intracellular binding site by (at least a part or fragment of) a cell wall, cell membrane or other layer (such as a lipid bilayer).

As described herein, the chimeric protein of the invention that is present in said composition preferably has the following overall structure (from the N-terminus to the C-terminus):

[N-terminal sequence]-[TM1]-[IC1]-[TM2]-[EC1]-[TM3]-[IC2]-[TM4]-[EC2]-[TM5]-[IC3]-[TM6]-[EC3]-[TM7]-[C-terminal sequence].

Preferably, the chimeric protein of the invention is such that its TMs can take on the barrel-like structure that is characteristic for (the 7TMs in) a naturally occurring GPCR, at least under the conditions that are applied when the chimeric GPCR of the invention is used for assay or screening purposes. Reference is again made to the prior art cited herein.

Preferably, in the chimeric protein that is present in said composition:
the extracellular loops that are present in the chimeric protein form a functional ligand binding site (optionally together with one or more of the TMs); and
the intracellular loops that are present in the chimeric protein form a functional ligand binding site to which the binding domain or binding unit can bind, and in particular functional ligand binding site that is conformation-dependent (as described herein).

Also, preferably, in the chimeric protein that is present in said composition, the TMs that are present in the chimeric protein are all derived (or essentially derived, as further described herein) from the same GPCR. More preferably, the TMs that are present in the chimeric protein are derived (or essentially derived, as further described herein) from the same (first) GPCR from which the extracellular loops are derived.

Also, preferably, the extracellular loops that are present in the chimeric protein have no more than 2, preferably no more than 1, and more preferably no amino acid differences with the extracellular loops of the first GPCR from which said extracellular loops have been derived.

As described herein, when the invention is to be used to identify, select, generate, test or develop ligands or compounds that are intended for therapeutic and/or prophylactic use in human beings, the parts of the sequence of the chimeric protein of the invention that are derived from the first GPCR and the parts of the sequence of the chimeric protein of the invention that are derived from the second GPCR are preferably both derived from GPCRs that are present in the human body.

Also, preferably, the extracellular loops that are present in the chimeric protein have no more than 2, preferably no more than 1, and more preferably no amino acid differences (as defined herein) with the extracellular loops of the first GPCR from which said extracellular loops have been derived. In addition, preferably, the intracellular loops that are present in the chimeric protein have no more than 2, preferably no more than 1, and more preferably no amino acid differences with the intracellular loops of the second GPCR from which said intracellular loops have been derived.

Furthermore, preferably, each of the TMs that is present in the chimeric protein has at least 80%, more preferably at least 85%, such as at least 90%, for example more than 95% and up to and including 100%, sequence identity with the amino acid sequence of the corresponding TM from the naturally occurring GPCR from which said TM has been derived (taking into account any amino acid residues that are the same as those present in and/or that are derived from the second GPCR in positions next to the ICLs, as further described herein). Also, preferably, each of the TMs that is present in the chimeric protein has no more than 7, preferably no more than 5, such as 5, 4, 3, 2, 1 or no amino acid differences (as defined herein) with the amino acid sequence of the corresponding TM from the naturally occurring GPCR from which said TM has been derived (in this case, not taking into account any amino acid residues that are the same as those present in and/or that are derived from the second GPCR in positions next to the ICLs, as further described herein).

Also, preferably, when the chimeric protein contains amino acid residues that are the same as those present in and/or that are derived from the second GPCR in positions next to the ICLs (also referred to herein as “ICL-flanking residues”), then said chimeric protein preferably contains, next to each ICL (i.e. in positions immediately adjacent to the first amino acid residue of the relevant ICL or last amino acid residue of the relevant ICL, respectively) no more than 10, preferably no more than 7, such as no more than 5, such as 5, 4, 3, 2 or 1 such ICL-flanking residues. Also, preferably, any such ICL-flanking residues (if present) will be same or essentially the same as the amino acid residues that flank the relevant ICL in the second GPCR from which said ICL has been derived. Also, any such ICL-flanking residues derived from the second GPCR will, if they are present in the chimeric GPCR, preferably be contiguous with the amino acid sequence of the relevant ICL from the second GPCR.

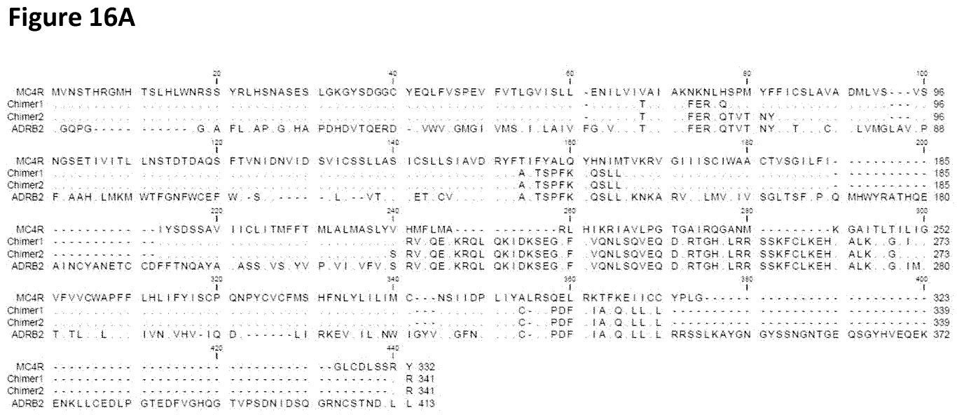

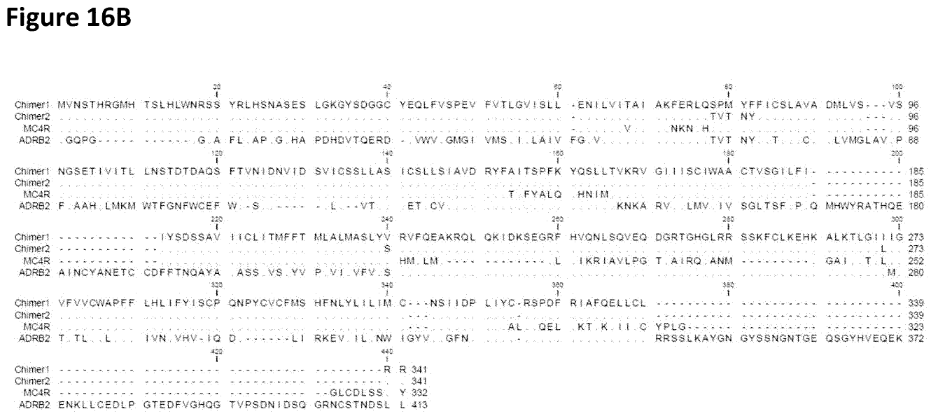

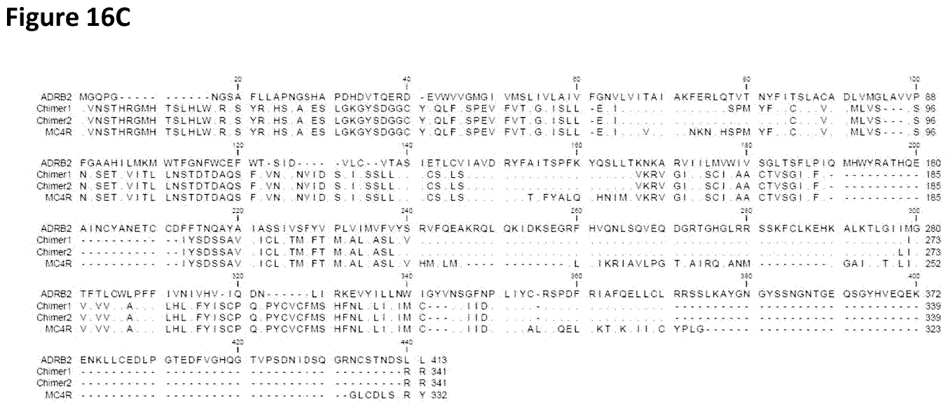

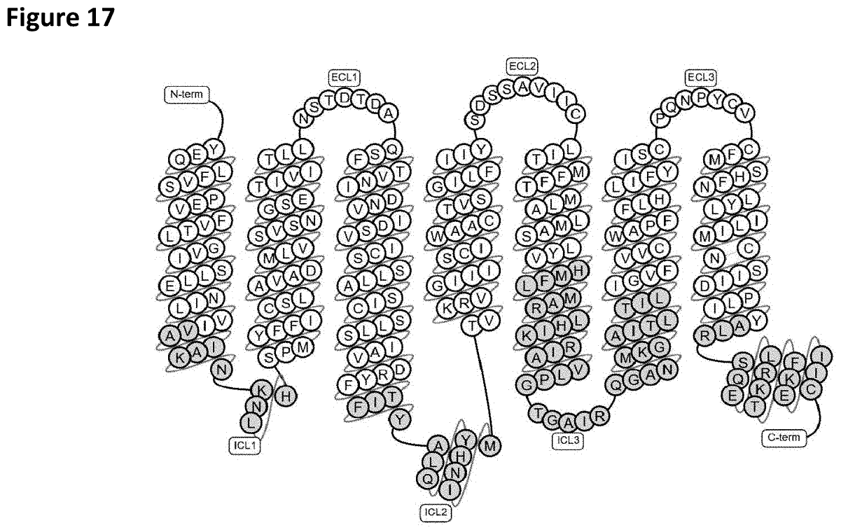

Overall, this means that, schematically represented, said ICL and any ICL-flanking residues taken from the second GPCR will have the following structure (indicated in bold/underline) from N-terminal end to C-terminal end):

[7TM]-[ICL-flanking residues, if any]-[ICL]-[ICL-flanking residues, if any]-[7TM].

in which the amino acid sequences from the relevant 7TMs that (in turn) flank the ICL-flanking residues are taken from the first GPCR. Reference is for example also made to the non-limiting FIGS. 16A-16C and 17, which give examples of chimeric GPCRs of the invention.

Preferably, when taken together, each ICL and any ICL-flanking residues will essentially have no amino acid differences (as defined herein) with the corresponding part of the amino acid sequence in the second GPCR from which the relevant ICL and the ICL-flanking residues have been derived. However, in the chimeric proteins of the invention, it also possible that each stretch of amino acid residues that is formed by an ICL and any ICL-flanking residues taken from the second GPCR has some amino acid differences (including substitutions, mutations or deletions) with the corresponding stretch of amino acid residues in the second GCPR from which said part of the sequence has been derived, but preferably no more than 5 amino acid differences, such as 5, 4, 3, 2 or 1 amino acid differences for each such stretch of ICL-flanking residues and ICL. And also, as described herein, each of the ICLs themselves will preferably have no more than 2, more preferably no more than 1, and most preferred no amino acid differences (as defined herein) with the intracellular loops of the (second) GPCR from which said intracellular loops have been derived.

It should also be noted that, for each ICL, whether any ICL-flanking residues are present or not, on which side of the ICL such ICL-flanking residues are present (i.e. on the N-terminal end, on the C-terminal end, or both), how many ICL-flanking residues are present (if any), and whether or not the stretch amino acid residues that is formed by each ICL and any ICL-flanking residues contains any amino acid differences with the corresponding stretch of amino acid residues in the second GPCR (and if so, how many amino acid differences and which amino acid differences, and where in the sequence), can each time be independently chosen for each of the ICLs.

Also, although it is usually preferred that any ICL-flanking residues replace the amino acid residues on the corresponding positions of the TM to which the relevant ICL is linked, it is also possible that the stretch of amino acid residues that is formed by each ICL and any ICL-flanking residues flanking said ICL is suitably inserted into the sequence of the first GPCR, with the ICL from the second GPCR replacing the corresponding ICL from the first GPCR and any ICL-flanking residues from the second GPCR either being inserted in the sequence or replacing some (but not all) of the amino acid residues in the TM that, in the first GPCR, flank the relevant ICL. Also, any amino acid differences that, in the final sequence of the chimeric GPCR, are present in the part(s) of the sequence that are formed by the each of the ICLs and its ICL flanking sequences (if any) can be derived from the amino acid sequence of the first GPCR (for example, because one or more of the ICL-flanking amino acid residues from the second GPCR are replaced by the amino acid residues that are present on the corresponding positions in the amino acid sequence of the first GPCR), but it is also possible that the final sequence of the chimeric GPCR contains one or more other amino acid differences at these positions (or a suitable combination of one or more amino acid differences derived from the first GPCR and one or more other amino acid differences).

It will also be clear to the skilled person that some of the stretches of amino acid residues in a GPCR that flank each of the ICLs contain amino acid residues are certain positions that are highly conserved. Reference is made to Table A above, which lists some of the so-called “signature residues” within Family A GPCRs.

It will be clear to the skilled person that preferably, such highly conserved amino acid residues in positions close to the ICLs will preferably also be conserved in the chimeric GCPRs of the invention, in particular when said conserved amino acid residues are also present in both the first and second GPCRs. Thus, in one aspect, a chimeric GPCR will preferably contain one or more, such as at least 5, preferably at least 10, more preferably at least 15, such as 15, 16, 17, 18, 19, 20 or all 21 of the signature residues listed in Table A above, in a suitable combination.

Preferably, a chimeric GPCR of the invention contains at least the following amino acid residues at the indicated positions:
G at position 1.49 and N at position 1.50;
L at position 2.46 and A at position 2.47;
D at position 3.49 and R at position 3.50;
W at position 4.50;
P at position 5.50 and Y at position 5.58;
F at position 6.44, C at position 6.47 and P at position 6.50;
   and preferably further at least 1 (such as 1, 2, 3, 4, 5, 6, 7, 8 or 9), more preferably at least 5 (such as 5, 6, 7, 8 or 9) of the further signature amino acid residues listed in Table A at the relevant positions in the sequence.

Restated in terms of position relative to the human β₂AR (UniProt P07550 (ADRB2_HUMAN), preferably, a chimeric GPCR of the invention contains at least the following amino acid residues at the indicated positions:
G at position 50 and N at position 51 relative to ADRB2_HUMAN;
L at position 75 and A at position 76 relative to ADRB2_HUMAN;
D at position 130 and R at position 131 relative to ADRB2_HUMAN;
W at position 158 relative to ADRB2_HUMAN;
P at position 211 and Y at position 219 relative to ADRB2_HUMAN;
F at position 282, C at position 285 and P at position 288 relative to ADRB2_HUMAN; and preferably further at least 1 (such as 1, 2, 3, 4, 5, 6, 7, 8 or 9), more preferably at least 5 (such as 5, 6, 7, 8 or 9) of the further signature amino acid residues listed in Table A at the relative relevant positions in the sequence.

In order to provide the sequence of a chimeric GPCR of the invention, the ICLs (and optionally also some ICL-flanking residues) in the amino acid sequence of the first GPCR should be replaced by the ICLs (and optionally also some ICL-flanking residues) from the second GPCR. This can be done using techniques of recombinant DNA known per se. Also, based on the information provided herein, the amino acid sequence of a chimeric GPCR can be designed (for example, taking the amino acid sequences of the first and second GPCR or an alignment of these sequences as a starting point) after which the corresponding chimeric GPCR can generated by synthesizing a nucleotide sequence that encodes said chimeric GPCR and by expressing said nucleotide sequence in a suitable host organism, again using techniques of recombinant DNA known per se.

Irrespective of how a chimeric GPCR of the invention is provided (i.e. the specific manner in which the ICLs and optionally any ICL-flanking residues from the first GPCRs are replaced with the ICLs and optionally any ICL-flanking residues from the second GPCR), preferably a chimeric GPCR of the invention is such that:
a) the amino acid residues that form ICL1 in the first GPCR and optionally one or more of the further amino acid residues that are present at the positions in the amino acid sequence of the first GPCR that lie between (and including) positions 1.49 and 1.50 (the positions relative to positions 50 and 51 of ADRB2_HUMAN), which as mentioned in most cases are GN; and position 2.50 (the position relative to position 79 of ADRB2_HUMAN), which as mentioned in most cases is D; are (suitably) replaced by the amino acid residues that form ICL1 in the second GPCR and optionally one or more of the further amino acid residues that are present at the positions in the amino acid sequence of the second GPCR that lie between (and including) positions 1.49 and 1.50 and position 2.50; or
b) the amino acid residues that form ICL2 in the first GPCR and optionally one or more of the further amino acid residues that are present at the positions in the amino acid sequence of the first GPCR that lie between (and including) positions 3.49 and 3.50 (the positions relative to positions 130 and 131 of ADRB2_HUMAN), which as mentioned in most cases are DR; and position 4.50 (the position relative to position 158 of ADRB2_HUMAN), which as mentioned in most cases is W; are (suitably) replaced by the amino acid residues that form ICL2 in the second GPCR and optionally one or more of the further amino acid residues that are present at the positions in the amino acid sequence of the second GPCR that lie between (and including) positions 3.49 and 3.50 and positions 4.50; or
c) the amino acid residues that form ICL3 in the first GPCR and optionally one or more of the further amino acid residues that are present at the positions in the amino acid sequence of the first GPCR that lie between (and including) positions 5.50 (the position relative to position 211 of ADRB2_HUMAN), which as mentioned in most cases is P; and position 6.50 (the position relative to position 288 of ADRB2_HUMAN), which as mentioned in most cases is P; are (suitably) replaced by the amino acid residues that form ICL3 in the second GPCR and optionally one or more of the further amino acid residues that are present at the positions in the amino acid sequence of the second GPCR that lie between (and including) position 5.50 and position 6.50;
   and preferably such that at least a) and b), at least a) and c) or at least b) and c) apply, and most preferably such that all of a), b) and c) apply.

Based on the disclosure herein and on an alignment and comparison between the amino acid sequence of the first GPCR and the amino acid sequence of the second GPCR, the skilled person will be able to select one or more ICL-flanking residues in the first GPCR that (in addition to the relevant ICL) can be suitably “replaced” by one or more ICL-flanking residues from the second GPCR, optionally after a limited degree of trial-and-error. For example and without limitation, from an alignment between the amino acid sequence of the first GPCR and the second GPCR, the skilled person can derive amino acid residues and/or positions that are the same and/or appear to be conserved between the amino acid sequences of the first and second GPCRs and such residues/positions can be used to guide the replacement/insertions of the ICLs and any ICL-flanking residues. For example and without limitation, based on a comparison of about 60 GPCR sequences from the GPCR database (https://gpcrdb.org/), it would appear that positions 1.52, 1.53 (the positions relative to positions 53 and 54 of ADRB2_HUMAN), the LA-motif at positions 2.46 and 2.47 (the positions relative to positions 75 and 76 of ADRB2_HUMAN) and positions 6.44 and 6.47 (the positions relative to positions 282 and 285 of ADRB2_HUMAN), which positions 6.44 and 6.47 together with the P at position 6.50 may form a FxxCxxP motif) may be conserved between a given first GPCR and a given second GPCR and such conserved residues may guide the replacement/insertions of the ICLs and any ICL-flanking residues.

It should also be noted that the stretch of amino acid residues from the second GPCR and the (corresponding) stretch of amino acid residues from the first GPCR that is “replaced” by said stretch of amino acid residues from the second GPCR do not need to be of the same length, in particular when it comes to ICL3, which is known to vary in length between different GPCRs, and even between GPCRs from the same family.

It is generally envisaged that the chimeric proteins of the invention, in combination with the ISVDs that are specific for the ICLs that are present in said chimeric proteins (which ISVDs are as further described herein), can find various uses, in particular in applications in which combinations of a GPCR and a conformation-inducing binding domain or binding unit (and in particular, a combination of a GPCR and a conformation-inducing ISVD) are being used. These include, but are not limited to, the various applications and uses described herein and in WO2012/007593, WO2012/007594, WO 2012/175643, WO 2014/118297, WO2014/122183 and WO 2014/118297. As further described herein, such applications and uses also include the use in the methods and arrangements that are as described in the co-pending US provisional application of assignee filed on Apr. 29, 2019 and entitled “Screening methods and assays for use with transmembrane proteins, in particular with GPCRs” and assignee's co-pending PCT application of the same title which has the same international filing date as the present application and invokes the same priority applications as the present application.

Further applications and uses will be clear to the skilled person based on the disclosure herein.

In particular, it is envisaged that the chimeric proteins of the invention, in combination with the conformation-inducing binding domains or binding units that are specific for the ICLs that are present in said chimeric proteins, as well as cells, cell lines, cellular compositions, vesicles, liposome and other compositions that contain and where appropriate express such a chimeric protein (and preferably also such a binding domain or binding unit), will find applications and uses in various assay techniques and screening methods, more in particular to identify, screen for, generate, test and develop compounds and ligands that are specific for, that are directed against and/or that can be used to modulate the GPCR from which the ECLs (and usually also essentially all of the TMs, as described herein) have been derived. As such, the chimeric proteins of the invention, in combination with the conformation-inducing binding domain or binding units that are specific for the ICLs that are present in said chimeric proteins, can be used in such assay and screening methodologies as an alternative to (i.e. to replace) the (non-chimeric) GPCR (and the ISVD that is specific for the ICLs of such non-chimeric GPCR) that are used in such methods (such as those described in WO2012/007593, WO2012/007594, WO 2012/175643, WO 2014/118297, WO2014/122183 and WO 2014/118297). This means that, compared to using a naturally occurring GPCR and a conformation-inducing ISVD that is directed towards the ICLs of said naturally occurring GPCR, the invention provides the skilled person with an alternative route to providing assay and screening methods for a desired naturally occurring GPCR, which route does not require that conformation-inducing ISVDs have to be raised against (the ICLs of) said naturally occurring GPCR, thus avoiding any practical issues or technical limitations (as mentioned herein) that may be associated with the need to do so. It is also envisaged that in some cases, replacing the ICLs of a desired naturally occurring GPCR with the ICLs of another GPCR may result in a chimeric GPCR that is more practical to work with (e.g. in terms of expression, folding, purification and/or stability) than the original non-chimeric GPCR, in particular under the conditions used for assay and screening techniques.

Thus, overall, it is envisaged that the invention will not only provide the skilled person with an alternative route towards establishing assay and screening methods involving GPCRs (which alternative methods may even in some respects be more practical or easier to establish or implement than the corresponding methods involving the use of the corresponding naturally occurring GPCRs), but may also make it possible to establish assay and screening methods for GPCRs that are currently essentially not possible or difficult to attain with naturally occurring or non-chimeric GPCR due to potential issues of technical feasibility.

The binding domain or binding unit that is used in the invention should be able to bind (and should, together with the ICLs, be chosen to be able to bind) to at least one of the ICLs in the chimeric GPCR of the invention, and preferably to at least two, such as essentially to all three ICLs. In particular, binding domain or binding unit that is used in the invention should bind, and preferably specifically bind, to the intracellular binding site (as defined herein) on the chimeric GPCR, which intracellular binding site may comprise 1, 2 or essentially all such ICLs.

Preferably, the binding domain or binding unit is such that it is capable of stabilizing and/or inducing a functional and/or active conformational state of the chimeric GPCR upon binding to the chimeric GPCR (i.e. to the intracellular binding site on the chimeric GPCR). Such a binding domain or binding unit is also referred to herein as a “conformation-inducing” binding domain or binding unit or a “conformation-stabilizing” binding domain or binding unit (which terms are used interchangeably herein). In particular, such a conformation-inducing binding domain or binding unit may be such that it is capable of stabilizing and/or inducing a druggable (as defined herein) conformational state of the chimeric GPCR upon binding to the chimeric GPCR (i.e. to the intracellular binding site on the chimeric GPCR).

Generally, this means that a conformation-inducing binding domain or binding unit will be specific for at least one functional conformational state of the chimeric GPCR (i.e. compared to at least one other, non-functional conformational state of the chimeric GPCR) and/or specific for at least one active or more active conformational state of the chimeric GPCR (i.e. compared to at least one inactive or less active conformational state of the chimeric GPCR). Preferably, a conformation-inducing binding domain or binding unit will be specific for at least one druggable conformational state of the chimeric GPCR (i.e. compared to at least one other conformational state of the chimeric GPCR that is not or less druggable).

In particular, a conformation-inducing binding domain or binding unit may be such that it preferentially binds to the chimeric GPCR of the invention (i.e. to the intracellular binding site as defined herein) when said chimeric GPCR is bound by an agonist-bound, i.e. such that it preferentially binds to the conformation(s) that the chimeric GPCR of the invention adopts when it is bound by an agonist (i.e. compared to binding to at least one conformation that the chimeric GPCR of the invention adopts when it is not bound by an agonist and/or when it is bound by an inverse agonist and/or an antagonist).

Also, a conformation-inducing binding domain or binding unit is preferably such that is enhances the affinity of the chimeric GPCR for an agonist, more preferably at least twofold, and even more preferably at least fivefold, such as at least tenfold.

Also, without being limited to any specific hypothesis or mechanism, a conformation-inducing binding domain or binding unit is preferably such that, upon binding to the chimeric GPCR (i.e. to the intracellular binding site as defined herein) it is capable of stabilizing and/or inducing the formation of a complex comprising the binding domain or binding unit, the chimeric GPCR and a compound or ligand that binds to the extracellular binding site (as defined herein) of the chimeric GPCR. In particular, the binding domain or binding unit may be such that, upon binding to the chimeric GPCR (i.e. to the intracellular binding site as defined herein) it is capable of stabilizing and/or inducing the formation of a complex comprising the binding domain or binding unit, the chimeric GPCR and an agonist that binds to the extracellular binding site (as defined herein) of the chimeric GPCR.

Preferably, the conformation-inducing binding domain or binding unit is derived from an immunoglobulin. More preferably, the conformation-inducing binding domain or binding unit is an amino acid sequence that has an immuoglobulin fold and that comprises 4 framework regions and 3 complementary determining regions. More preferably, the conformation-inducing binding domain or binding unit is an immunoglobulin single variable domain, such as an ISVD that has been derived from a Camelid antibody, such as a VHH or Nanobody. The conformation-inducing binding domain or binding unit may also be a suitable fragment from such an immunoglobulin. ISVDs that are capable of inducing or stabilizing a functional, active and/or druggable conformational state of a GPCR (and/or that are specific for a functional, active and/or druggable conformational state of a GPCR) are for example known from WO2012/007593, WO2012/007594, WO 2012/175643, WO 2014/118297, WO2014/122183 and WO 2014/118297, and such ISVDs (which are also referred to as ConfoBodies) may be used in the present invention as a conformation-inducing binding domain or binding unit, in combination with a chimeric GPCR that contains the ICLs from the GPCR against which the relevant ConfoBody was raised (reference is again made to WO2012/007593, WO2012/007594, WO 2012/175643, WO 2014/118297, WO2014/122183 and WO 2014/118297).

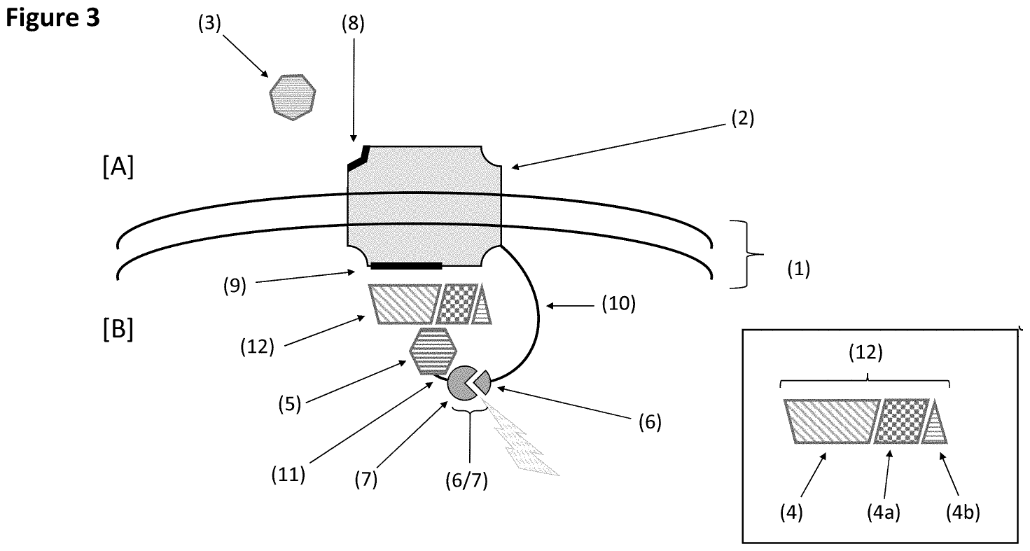

The binding domain or binding unit can be used as such (i.e. as a distinct binding protein, for example as a monovalent VHH) or it can be part of a large protein that contains one or more further amino acid sequences, binding domains or binding units. For example and without limitation, and as further described herein, when the chimeric protein of the invention and the binding domain or binding unit are used in the methods and arrangements that are as described in the co-pending US provisional application filed on Apr. 29, 2019 and entitled “Screening methods and assays for use with transmembrane proteins, in particular with GPCRs” referred to below, the binding domain or binding unit can form part of the “second fusion protein” that is used in said methods and arrangements. As further described herein, in such a second fusion protein, the binding domain or binding unit may be linked, directly or via a suitable spacer or linker, to a binding member that is part of a binding pair that can generate a detectable signal. A similar arrangement is also shown in FIG. 3 in the publication of Jacobs et al., Int. J. Mol. Sci., 2019, 20, 2597.

Also, as mentioned herein, in one aspect, the binding domain or binding unit may be fused to the chimeric protein of the invention, essentially as described in the International application WO 2014/118297.

The invention also relates to the use of such a binding domain or binding unit to induce a conformational change in a chimeric GPCR of the invention (as further described herein). In particular, the invention also relates to the use of such a binding domain or binding unit to induce a functional, active and/or druggable conformational state in a chimeric GPCR of the invention and/or to stabilize such a conformational state of a chimeric GPCR of the invention.

The invention further relates to the use of such a binding domain or binding unit to induce the formation of and/or to stabilize a complex that comprises said binding domain or binding unit and a chimeric GPCR of the invention. Such a complex may further comprise a ligand or compound that is bound to the extracellular binding site (as defined herein) on the chimeric GPCR (which ligand or compound can also be as further described herein, and can in particular be an agonist). In particular, the invention also relates to the use of such a binding domain or binding unit to induce the formation of and/or to stabilize such a complex in which the chimeric GPCR of the invention is in a functional, active, druggable conformational state. In one specific aspect, the invention also relates to the use of such a binding domain or binding unit to induce the formation of and/or to stabilize such a complex in which the chimeric GPCR of the invention is in a ligand-bound (and preferably an agonist-bound) conformational state in a chimeric GPCR of the invention (as further described herein).

In a further aspect, the invention relates to a complex comprising:
a) a chimeric protein comprising an N-terminal sequence, a C-terminal sequence, seven transmembrane domains (TM1 to TM7), three extracellular loops (EC1 to EC3) and three intracellular loops (IC1 to IC3), in which:
   the extracellular loops that are present in the chimeric protein are (essentially) derived from a first GPCR; and
   the intracellular loops that are present in the chimeric protein are (essentially) derived from a second GPCR;
      and:
b) a binding domain or binding unit that can specifically bind to (the binding site formed by) the intracellular loops that are present in said chimeric protein;
   and optionally:
c) a ligand or compound that is bound to the extracellular binding site (as defined herein) on the chimeric GPCR.

In a further aspect, the invention relates to such a complex that comprises all three of the chimeric GPCR referred to under a), the binding domain or binding unit as referred to under b) and the ligand or compound referred to under c).

Also, in any such complex, the binding domain or binding unit may be fused to the chimeric protein, essentially as described in the International application WO 2014/118297, and the invention also relates to complexes that comprise such a fusion protein and (optionally) a ligand or compound that is bound to the extracellular binding site (as defined herein) on the chimeric GPCR.

Preferably, the chimeric GPCR in said complex is in a functional conformational state and/or in an active conformational change. In particular, the chimeric GPCR in said complex may be in a druggable conformational change. Said functional, active and/or druggable conformational state may also be induced by the binding of the binding domain or binding unit referred to under b) to the chimeric GPCR, by the binding of the ligand or compound referred to under c) to the chimeric GPCR, and/or by the binding of both said binding domain or binding unit and compound or ligand to the chimeric GPCR. In one aspect, said functional, active and/or druggable conformational state is a conformational state that is induced by the binding of an agonist to the chimeric GPCR (with said agonist being the ligand or compound referred to under c)), optionally together with the binding of a binding domain or binding unit as referred to under b) to the chimeric GPCR.

The binding domain or binding unit that is present in the complex is again preferably a conformation-inducing (as defined herein) binding domain or binding unit, i.e. a binding domain or binding unit that is capable of stabilizing and/or inducing a functional and/or active conformational state of the chimeric GPCR upon binding to the chimeric GPCR (i.e. to the intracellular binding site on the chimeric GPCR). More preferably, the binding domain or binding unit will be capable of inducing the formation of the complex that is formed by the chimeric protein referred to under a), the binding domain or binding unit referred to under b) and the ligand or compound referred to under c), and/or will be capable of stabilizing such a complex.

The ligand or compound that is present in said complex is preferably a full agonist, a partial agonist, an inverse agonist or an antagonist, and is more preferably a full agonist or partial agonist. In particular, said ligand or compound may be a small molecule, a protein, a peptide, a protein scaffold, a nucleic acid, an ion, a carbohydrate or an antibody, or any suitable fragment thereof.

In a further aspect, the complex formed by the chimeric GPCR referred to under a), the binding domain or binding unit referred to under b) and (optionally) the ligand or compound referred to under c) is bound to and/or immobilized on a suitable solid support. Such a complex may for example be formed by first forming a complex of the invention that only comprises the chimeric GPCR referred to under a) and the binding domain or binding unit referred to under b), which complex is bound to a solid support, and then contacting said complex with the ligand or compound referred to under c), which may be present in a suitable (preferably liquid and usually aqueous) medium. In one specific aspect, such a complex is formed by performing the following steps (performed in the indicated order):
providing a binding domain or binding unit as referred to under b) above that is immobilized on a suitable solid support;
contacting said immobilized binding domain or binding unit with a chimeric GCPR of the invention (i.e. under conditions such that the immobilized binding domain or binding unit captures said chimeric GPCR, and preferably such that the immobilized binding domain or binding unit captures said chimeric GPCR in a functional and/or active conformational state, and more preferably in a druggable conformational state); and
contacting the complex formed by the immobilized binding domain or binding unit and the captured chimeric GPCR with a compound or ligand as referred to under c) above.

The binding domain or binding unit is again preferably a conformation-inducing binding domain or binding unit (as defined herein) and again preferably an ISVD (and preferably a conformation-inducing ISVD).

Suitable solid supports and immobilisation techniques will be clear to the skilled person and for example include beads, columns, slides, chips or plates. Reference is for example made to WO 2012/007593, pages 55 to 57.

In another aspect, the invention relates to a solid support onto which is immobilized a complex comprising a chimeric GPCR as referred to under a), a binding domain or binding unit as referred to under b) and optionally a ligand or compound as referred to under c).

The invention also relates to uses of a solid support onto which is immobilized a complex comprising a chimeric GPCR as referred to under a) and a binding domain or binding unit as referred to under b). In particular, the invention relates to the use of such a solid support in:
identifying, generating and/or screening for ligands or compounds that can bind (and in particular, specifically binding as defined herein) to the chimeric GPCR that is present in said complex (e.g. using standard screening techniques known per se); and/or
determining at least one property of a compound or ligand, such as its ability to bind (and in particular, specifically bind as defined herein) to the chimeric GPCR that is present in said complex and/or to modulate said chimeric GPCR (e.g. using standard assay techniques known per se).

The invention also relates to a method for determining at least one property of a compound or ligand, said method comprising at least the steps of:
providing a complex of a chimeric GPCR of the invention and a binding domain or binding unit as described herein;
contacting said complex with said compound or ligand.

Said method preferably also comprises the step of measuring (the change in) at least one signal or parameter that is representative for said at least one property. As described herein, said property can be the ability to bind (and in particular the ability to specifically bind) to the chimeric GPCR of the invention and/or to said complex. Said at least one property can also be the ability to modulate said chimeric GPCR, for example the ability to act as an agonist (e.g. a partial or a full agonist) of the chimeric GPCR, the ability to act as an antagonist of the chimeric GPCR as an antagonist, and/or the ability to act as an inverse agonist of the chimeric GPCR. Again, preferably, said at least one property as determined using a chimeric GPCR of the invention is representative for essentially the same or an essentially similar property of the naturally occurring GPCR from which the ECLs (and preferably essentially also the TMs, as further described herein) have been derived.

Again, in said method, the complex may be immobilized on a solid support. Also, the binding domain or binding unit is preferably a conformation-inducing (as defined herein) binding domain or binding unit and more preferably a conformation-inducing ISVD (such as a ConfoBody). Also, preferably, in said complex, the chimeric GPCR of the invention is in a functional, active and/or druggable state.

Also, based on the disclosure herein, it will also be clear to the skilled person that, when the chimeric GPCR of the invention and the binding domain and binding unit are provided as a fusion protein (e.g. as described in the International application WO 2014/118297), a complex as described herein may also be formed by the chimeric GPCR that is present in said fusion protein and the binding domain or binding unit that is present in said fusion protein, optionally together with a compound or ligand as referred to under c).

In another aspect, the invention relates to a method of identifying and/or generating compounds or ligands that are capable of binding to an extracellular binding site (as defined herein) of a GPCR, said method comprising the steps of:
a) providing a chimeric GPCR that essentially comprises (at least) the extracellular binding site of said GPCR and that comprises the intracellular loops of another GPCR;
b) providing a binding domain or binding unit that can bind to a binding site on said chimeric GPCR that comprises at least one of said intracellular loops;
c) contacting said chimeric GPCR with said binding domain or binding unit under conditions that allow said binding domain or binding unit to bind to said binding site on the chimeric GPCR that comprises at least one of said intracellular loops;
d) contacting said chimeric GPCR with one or more test compounds or ligands under conditions that allow said test compounds to bind to the extracellular binding site of said chimeric GPCR;
e) evaluating whether each of the test compounds or ligands (and/or which of said test compounds or ligands) binds to the chimeric GPCR in the presence of said binding domain or binding unit; and optionally
f) selecting the test compounds or ligands that bind to the chimeric GPCR in the presence of said binding domain or binding unit.

In said methods, the chimeric GPCR and the binding domain or binding unit are preferably again as further described herein (with any preferences or preferred aspects described herein for a chimeric GPCR of the invention and/or for such a binding domain/binding unit also being preferred for use in said method). Also, again, the chimeric GPCR that is provided and used in the above steps is preferably such, and the conditions under which said chimeric GPCR and the binding domain or binding unit are used in the above steps are preferably chosen such, that binding of the test compounds to the chimeric GPCR under the conditions used is representative for binding of said test compound(s) to said GPCR. Also, in such a method, the chimeric GPCR and the binding domain or binding unit may be present in a suitable cellular composition and/or expressed by a suitable cell or cell line or they may be present in a suitable liposome or vesicle, all as further described herein. The chimeric GPCR or the binding domain or binding unit may also be immobilized on a solid support, again as further described herein. The chimeric GPCR or the binding domain or binding unit may also be suitably provided and used as a fusion protein, as further described herein and in the International application WO 2014/118297.

It should also be noted that, as mentioned herein and as is known per se for naturally occurring GPCRs (reference is for example again made to Eglen and Reisine, cited herein), a chimeric GPCR of the invention may, in addition to the binding site that corresponds to the orthosteric binding site of the first GPCR, also contain one or more allosteric sites that correspond to one or more allosteric binding sites on the first GPCR, depending on the ECLs and TMs that are present in the chimeric GPCR of the invention. Thus, it is expected that, more generally, the present invention can also be used to identify, generate, screen for, test and/or develop compounds and ligands that act as orthosteric binders for the first GPCR as well as compounds and ligands that act as allosteric binders for the first GPCR.

Therefore, in a further aspect, the invention relates to a method of identifying and/or generating compounds or ligands that are capable of binding to a GPCR, said method comprising the steps of.
a) providing a chimeric GPCR that comprises the extracellular loops of said GPCR and the TMs of said GPCR (or essentially all of the TMs of said GPCR, as further described herein) and that comprises the intracellular loops of another GPCR;
b) providing a binding domain or binding unit that can bind to a binding site on said chimeric GPCR that comprises at least one of said intracellular loops;
c) contacting said chimeric GPCR with said binding domain or binding unit under conditions that allow said binding domain or binding unit to bind to said binding site on the chimeric GPCR that comprises at least one of said intracellular loops;
d) contacting said chimeric GPCR with one or more test compounds or ligands under conditions that allow said test compounds to bind to (at least) the extracellular loops of said chimeric GPCR;
e) evaluating whether each of the test compounds or ligands (and/or which of said test compounds or ligands) binds to the chimeric GPCR in the presence of said binding domain or binding unit; and optionally
f) selecting the test compounds or ligands that bind to the chimeric GPCR in the presence of said binding domain or binding unit.

In a further aspect, the invention relates to a method of identifying and/or generating compounds or ligands that are capable of binding to an extracellular binding site of a GPCR, said method comprising the steps of:
a) providing a chimeric GPCR that comprises the extracellular loops of said GPCR and the TMs of said GPCR (or essentially all of the TMs of said GPCR, as further described herein), such that said extracellular loops and said TMs form a functional extracellular binding site, and that further comprises the intracellular loops of another GPCR;
b) providing a binding domain or binding unit that can bind to a binding site on said chimeric GPCR that comprises at least one of said intracellular loops;
c) contacting said chimeric GPCR with said binding domain or binding unit under conditions that allow said binding domain or binding unit to bind to said binding site on the chimeric GPCR that comprises at least one of said intracellular loops;
d) contacting said chimeric GPCR with one or more test compounds or ligands under conditions that allow said test compounds to bind to (at least) the extracellular loops of said chimeric GPCR;
e) evaluating whether each of the test compounds or ligands (and/or which of said test compounds or ligands) binds to the extracellular binding site of said chimeric GPCR in the presence of said binding domain or binding unit; and optionally
f) selecting the test compounds or ligands that bind to the chimeric GPCR in the presence of said binding domain or binding unit.

In a further aspect, the invention relates to a method of identifying and/or generating compounds or ligands that are capable of binding to an extracellular binding site of a GPCR, said method comprising the steps of:
a) providing a cell or cell line that contains:
   a chimeric GPCR within its cell wall or cell membrane, in which said chimeric GPCR comprises (at least) the extracellular loops of said GPCR and in which said chimeric GPCR comprises the intracellular loops of another GPCR and in which said extracellular loops of the chimeric GPCR extend out (as defined herein) into the extracellular environment and in which said intracellular loops of the chimeric GPCR extend out into the intracellular environment of said cell or cell line;
   and that further contains:
   a binding domain or binding unit that can bind to a binding site on said chimeric GPCR that comprises at least one of said intracellular loops;
b) contacting said cell or cell line with one or more test compounds or ligands under conditions that allow said test compounds to bind to (at least) the extracellular loops of said chimeric GPCR;
c) evaluating whether each of the test compounds or ligands (and/or which of said test compounds or ligands) binds to said chimeric GPCR that is present in said cell or cell line; and optionally
d) selecting the test compounds or ligands that bind to said chimeric GPCR.

In this method of the invention, the cell or cell line that contains said chimeric GPCR and said binding domain or binding unit can in particular be provided by maintaining or culturing a cell or cell line that is capable of expressing said chimeric GPCR and said binding domain or binding unit under conditions such that said cell line expresses said chimeric GPCR (and in particular suitably expresses said chimeric GPCR, as defined herein) and also expresses said binding domain or binding unit.

In a further aspect, the invention relates to a method of identifying and/or generating compounds or ligands that are capable of binding to an extracellular binding site of a GPCR, said method comprising the steps of:
a) providing a vesicle or liposome that contains:
   a chimeric GPCR within its wall or membrane, in which said chimeric GPCR comprises (at least) the extracellular loops of said GPCR and in which said chimeric GPCR comprises the intracellular loops of another GPCR and in which said extracellular loops of the chimeric GPCR extend out (as defined herein) into the environment outside of the vesicle or liposome and in which said intracellular loops of the chimeric GPCR extend out into the environment within said vesicle or liposome;
   and that further contains:
   a binding domain or binding unit that can bind to a binding site on said chimeric GPCR that comprises at least one of said intracellular loops;
b) contacting said vesicle or liposome with one or more test compounds or ligands under conditions that allow said test compounds to bind to (at least) the extracellular loops of said chimeric GPCR;
c) evaluating whether each of the test compounds or ligands (and/or which of said test compounds or ligands) binds to said chimeric GPCR that is present in said vesicle or liposome; and optionally
d) selecting the test compounds or ligands that bind to said chimeric GPCR.

In another aspect, the invention relates to a method of identifying and/or generating compounds or ligands that are capable of binding to an extracellular binding site (as defined herein) of a GPCR, said method comprising the steps of:
a) providing a chimeric GPCR that essentially comprises (at least) the extracellular binding site of said GPCR and that comprises the intracellular loops of another GPCR;
b) contacting said chimeric GPCR with:
   a binding domain or binding unit that can bind to a binding site on said chimeric GPCR that comprises at least one of said intracellular loops; and with
   a test compound or ligand;
   under conditions that allow the formation of complex between said chimeric GPCR, said binding domain or binding unit and said test compound or ligand;
c) evaluating whether said test compound or ligand forms a complex with said chimeric GPCR and said binding domain or binding unit;
   and optionally:
d) selecting one or more test compounds or ligands that form a complex with said chimeric GPCR and said binding domain or binding unit.

In each of said methods, as well as the other methods described herein, the chimeric GPCR and the binding domain or binding unit are preferably as described herein (with any preferences or preferred aspects described herein for a chimeric GPCR of the invention and/or for such a binding domain/binding unit also being preferred for use in said methods). Also, as described herein, the chimeric GPCR and the binding domain or binding unit may also be suitably provided and used as part of a fusion protein, again essentially as described in WO 2014/118297.

In another aspect, the invention relates to a method of identifying compounds that are capable of binding to a functional conformational state of a GPCR, said method comprising the steps of:
a) providing a chimeric GPCR that essentially comprises (at least) the extracellular binding site of said GPCR and that comprises the intracellular loops of another GPCR;
b) providing a binding domain or binding unit that can bind to a binding site on said chimeric GPCR that comprises at least one of said intracellular loops, in which said binding domain or binding unit is a conformation-inducing (as defined herein) binding domain or binding unit (i.e. a binding domain or binding unit that is capable of stabilizing and/or inducing a functional and/or active conformational state of the chimeric GPCR upon binding to the chimeric GPCR);
c) contacting said chimeric GPCR with said binding domain or binding unit under conditions that allow said binding domain or binding unit to bind to said binding site on the chimeric GPCR;
d) contacting said chimeric GPCR with one or more test compounds or ligands under conditions that allow said test compounds to bind to the extracellular binding site of said chimeric GPCR;
e) evaluating whether each of the test compounds or ligands (and/or which of said test compounds or ligands) binds to the chimeric GPCR in the presence of said binding domain or binding unit; and optionally
f) selecting the test compounds or ligands that bind to the chimeric GPCR in the presence of said binding domain or binding unit.

In another aspect, the invention relates to a method of identifying compounds that are capable of binding to an active conformational state of a GPCR, said method comprising the steps of
a) providing a chimeric GPCR that essentially comprises (at least) the extracellular binding site of said GPCR and that comprises the intracellular loops of another GPCR;
b) providing a binding domain or binding unit that can bind to a binding site on said chimeric GPCR that comprises at least one of said intracellular loops, in which said binding domain or binding unit is a conformation-inducing (as defined herein) binding domain or binding unit (i.e. a binding domain or binding unit that is capable of stabilizing and/or inducing a functional and/or active conformational state of the chimeric GPCR upon binding to the chimeric GPCR);
c) contacting said chimeric GPCR with said binding domain or binding unit under conditions that allow said binding domain or binding unit to bind to said binding site on the chimeric GPCR that comprises at least one of said intracellular loops;
d) contacting said chimeric GPCR with one or more test compounds or ligands under conditions that allow said test compounds to bind to the extracellular binding site of said chimeric GPCR;
e) evaluating whether each of the test compounds or ligands (and/or which of said test compounds or ligands) binds to the chimeric GPCR in the presence of said binding domain or binding unit; and optionally
f) selecting the test compounds or ligands that bind to the chimeric GPCR in the presence of said binding domain or binding unit.

In another aspect, the invention relates to a method of identifying compounds that are capable of binding to a functional conformational state of a GPCR, said method comprising the steps of:
a) providing a chimeric GPCR that comprises the extracellular loops of said GPCR and the TMs of said GPCR (or essentially all of the TMs of said GPCR, as further described herein) and that comprises the intracellular loops of another GPCR;
b) providing a binding domain or binding unit that can bind to a binding site on said chimeric GPCR that comprises at least one of said intracellular loops, in which said binding domain or binding unit is a conformation-inducing (as defined herein) binding domain or binding unit;
c) contacting said chimeric GPCR with said binding domain or binding unit under conditions that allow said binding domain or binding unit to bind to said binding site on the chimeric GPCR that comprises at least one of said intracellular loops;
d) contacting said chimeric GPCR with one or more test compounds or ligands under conditions that allow said test compounds to bind to (at least) the extracellular loops of said chimeric GPCR;
e) evaluating whether each of the test compounds or ligands (and/or which of said test compounds or ligands) binds to the chimeric GPCR in the presence of said binding domain or binding unit; and optionally
f) selecting the test compounds or ligands that bind to the chimeric GPCR in the presence of said binding domain or binding unit.

In another aspect, the invention relates to a method of identifying compounds that are capable of binding to an active conformational state of a GPCR, said method comprising the steps of:
a) providing a chimeric GPCR that comprises the extracellular loops of said GPCR and the TMs of said GPCR (or essentially all of the TMs of said GPCR, as further described herein) and that comprises the intracellular loops of another GPCR;
b) providing a binding domain or binding unit that can bind to a binding site on said chimeric GPCR that comprises at least one of said intracellular loops, in which said binding domain or binding unit is a conformation-inducing (as defined herein) binding domain or binding unit;
c) contacting said chimeric GPCR with said binding domain or binding unit under conditions that allow said binding domain or binding unit to bind to said binding site on the chimeric GPCR that comprises at least one of said intracellular loops;
d) contacting said chimeric GPCR with one or more test compounds or ligands under conditions that allow said test compounds to bind to (at least) the extracellular loops of said chimeric GPCR;
e) evaluating whether each of the test compounds or ligands (and/or which of said test compounds or ligands) binds to the chimeric GPCR in the presence of said binding domain or binding unit; and optionally
f) selecting the test compounds or ligands that bind to the chimeric GPCR in the presence of said binding domain or binding unit.

In another aspect, the invention relates to a method of identifying compounds that are capable of binding to a functional conformational state of a GPCR, said method comprising the steps of
a) providing a cell or cell line that contains:
   a chimeric GPCR within its cell wall or cell membrane, in which said chimeric GPCR comprises (at least) the extracellular loops of said GPCR and in which said chimeric GPCR comprises the intracellular loops of another GPCR and in which said extracellular loops of the chimeric GPCR extend out (as defined herein) into the extracellular environment and in which said intracellular loops of the chimeric GPCR extend out into the intracellular environment of said cell or cell line;
   and that further contains:
   a binding domain or binding unit that can bind to a binding site on said chimeric GPCR that comprises at least one of said intracellular loops;
b) contacting said cell or cell line with one or more test compounds or ligands under conditions that allow said test compounds to bind to (at least) the extracellular loops of said chimeric GPCR;
c) evaluating whether each of the test compounds or ligands (and/or which of said test compounds or ligands) binds to said chimeric GPCR that is present in said cell or cell line; and optionally
d) selecting the test compounds or ligands that bind to said chimeric GPCR.

In this method of the invention that involves the use of a cell or cell line, said chimeric GPCR and said binding domain or binding unit can in particular be provided by maintaining or culturing a cell or cell line that is capable of expressing said chimeric GPCR and said binding domain or binding unit under conditions such that said cell line expresses said chimeric GPCR (and in particular suitably expresses said chimeric GPCR, as defined herein) and also expresses said binding domain or binding unit.

In another aspect, the invention relates to a method of identifying compounds that are capable of binding to an active functional conformational state of a GPCR, said method comprising the steps of:
a) providing a cell or cell line that contains:
   a chimeric GPCR within its cell wall or cell membrane, in which said chimeric GPCR comprises (at least) the extracellular loops of said GPCR and in which said chimeric GPCR comprises the intracellular loops of another GPCR and in which said extracellular loops of the chimeric GPCR extend out (as defined herein) into the extracellular environment and in which said intracellular loops of the chimeric GPCR extend out into the intracellular environment of said cell or cell line;
   and that further contains:
   a binding domain or binding unit that can bind to a binding site on said chimeric GPCR that comprises at least one of said intracellular loops;
b) contacting said cell or cell line with one or more test compounds or ligands under conditions that allow said test compounds to bind to (at least) the extracellular loops of said chimeric GPCR;
c) evaluating whether each of the test compounds or ligands (and/or which of said test compounds or ligands) binds to said chimeric GPCR that is present in said cell or cell line; and optionally
d) selecting the test compounds or ligands that bind to said chimeric GPCR.
   In this method of the invention, the cell or cell line that contains said chimeric GPCR and said binding domain or binding unit can in particular be provided by maintaining or culturing a cell or cell line that is capable of expressing said chimeric GPCR and said binding domain or binding unit under conditions such that said cell line expresses said chimeric GPCR (and in particular suitably expresses said chimeric GPCR, as defined herein) and also expresses said binding domain or binding unit.

In this method of the invention that involves the use of a cell or cell line, said chimeric GPCR and said binding domain or binding unit can in particular be provided by maintaining or culturing a cell or cell line that is capable of expressing said chimeric GPCR and said binding domain or binding unit under conditions such that said cell line expresses said chimeric GPCR (and in particular suitably expresses said chimeric GPCR, as defined herein) and also expresses said binding domain or binding unit.

In another aspect, the invention relates to a method of identifying compounds that are capable of binding to a functional conformational state of a GPCR, said method comprising the steps of:
a) providing a vesicle or liposome that contains:
   a chimeric GPCR within its wall or membrane, in which said chimeric GPCR comprises (at least) the extracellular loops of said GPCR and in which said chimeric GPCR comprises the intracellular loops of another GPCR and in which said extracellular loops of the chimeric GPCR extend out (as defined herein) into the environment outside of the vesicle or liposome and in which said intracellular loops of the chimeric GPCR extend out into the environment within said vesicle or liposome;
   and that further contains:
   a binding domain or binding unit that can bind to a binding site on said chimeric GPCR that comprises at least one of said intracellular loops;
b) contacting said vesicle or liposome with one or more test compounds or ligands under conditions that allow said test compounds to bind to (at least) the extracellular loops of said chimeric GPCR;
c) evaluating whether each of the test compounds or ligands (and/or which of said test compounds or ligands) binds to said chimeric GPCR that is present in said vesicle or liposome; and optionally
d) selecting the test compounds or ligands that bind to said chimeric GPCR.

In another aspect, the invention relates to a method of identifying compounds that are capable of binding to an active conformational state of a GPCR, said method comprising the steps of:
a) providing a vesicle or liposome that contains:
   a chimeric GPCR within its wall or membrane, in which said chimeric GPCR comprises (at least) the extracellular loops of said GPCR and in which said chimeric GPCR comprises the intracellular loops of another GPCR and in which said extracellular loops of the chimeric GPCR extend out (as defined herein) into the environment outside of the vesicle or liposome and in which said intracellular loops of the chimeric GPCR extend out into the environment within said vesicle or liposome;
   and that further contains:
   a binding domain or binding unit that can bind to a binding site on said chimeric GPCR that comprises at least one of said intracellular loops;
b) contacting said vesicle or liposome with one or more test compounds or ligands under conditions that allow said test compounds to bind to (at least) the extracellular loops of said chimeric GPCR;
c) evaluating whether each of the test compounds or ligands (and/or which of said test compounds or ligands) binds to said chimeric GPCR that is present in said vesicle or liposome; and optionally
d) selecting the test compounds or ligands that bind to said chimeric GPCR.

In another aspect, the invention relates to a method of identifying compounds that are capable of binding to a functional conformational state of a GPCR, said method comprising the steps of:
a) providing a chimeric GPCR that essentially comprises (at least) the extracellular binding site of said GPCR and that comprises the intracellular loops of another GPCR;
b) contacting said chimeric GPCR with:
   a binding domain or binding unit that can bind to a binding site on said chimeric GPCR that comprises at least one of said intracellular loops; and with
   a test compound or ligand;
   under conditions that allow the formation of complex between said chimeric GPCR, said binding domain or binding unit and said test compound or ligand;
c) evaluating whether said test compound or ligand forms a complex with said chimeric GPCR and said binding domain or binding unit;
   and optionally:
d) selecting one or more test compounds or ligands that form a complex with said chimeric GPCR and said binding domain or binding unit.

In another aspect, the invention relates to a method of identifying compounds that are capable of binding to a functional conformational state of a GPCR, said method comprising the steps of:
a) providing a chimeric GPCR that essentially comprises (at least) the extracellular binding site of said GPCR and that comprises the intracellular loops of another GPCR;
b) contacting said chimeric GPCR with:
   a binding domain or binding unit that can bind to a binding site on said chimeric GPCR that comprises at least one of said intracellular loops; and with
   a test compound or ligand;
   under conditions that allow the formation of complex between said chimeric GPCR, said binding domain or binding unit and said test compound or ligand;
c) evaluating whether said test compound or ligand forms a complex with said chimeric GPCR and said binding domain or binding unit;
   and optionally:
d) selecting one or more test compounds or ligands that form a complex with said chimeric GPCR and said binding domain or binding unit.

Again, in all these methods, the chimeric GPCR and the binding domain or binding unit are preferably again as further described herein (with any preferences or preferred aspects described herein for a chimeric GPCR of the invention and/or for such a binding domain/binding unit also being preferred for use in said method). Also, again, the chimeric GPCR that is provided and used in the above steps is preferably such, and the conditions under which said chimeric GPCR and the binding domain or binding unit are used in the above steps are preferably chosen such, that binding of the test compounds to the chimeric GPCR under the conditions used is representative for binding of said test compound(s) to the GPCR from which the extracellular binding site has been derived. Also, in such a method, the chimeric GPCR and the binding domain or binding unit may be present in a suitable cellular composition and/or expressed by a suitable cell or cell line or they may be present in a suitable liposome or vesicle, all as further described herein. The chimeric GPCR or the binding domain or binding unit may also be immobilized on a solid support, again as further described herein. The chimeric GPCR or the binding domain or binding unit may also be suitably provided and used as a fusion protein, as further described herein and in the International application WO 2014/118297.

The invention further relates to a method of identifying and/or generating compounds or ligands that are capable of binding to an extracellular binding site (as defined herein) of a GPCR, said method comprising the steps of
a) providing a composition that comprises (i) a chimeric GPCR that essentially comprises (at least) the extracellular binding site of said GPCR and that comprises the intracellular loops of another GPCR; and (ii) a binding domain or binding unit that can bind to a binding site on said chimeric GPCR that comprises at least one of said intracellular loops;
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR;
c) evaluating whether each of the test compounds or ligands (and/or which of said test compounds or ligands) binds to the chimeric GPCR; in said composition; and optionally
d) selecting the test compounds or ligands that bind to the chimeric GPCR in said composition.

The invention also relates to a method of identifying and/or generating compounds or ligands that are capable of binding to an extracellular binding site (as defined herein) of a GPCR, said method comprising the steps of:
a) providing a composition that comprises (i) a chimeric GPCR that comprises the extracellular loops of said GPCR and the TMs of said GPCR (or essentially all of the TMs of said GPCR, as further described herein) and that comprises the intracellular loops of another GPCR; and (ii) a binding domain or binding unit that can bind to a binding site on said chimeric GPCR that comprises at least one of said intracellular loops;
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR;
c) evaluating whether each of the test compounds or ligands (and/or which of said test compounds or ligands) binds to the chimeric GPCR; in said composition; and optionally
d) selecting the test compounds or ligands that bind to the chimeric GPCR in said composition.

The invention also relates to a method of forming a complex of a chimeric GPCR, a binding domain or binding unit, and a compound or ligand that is capable of binding to an extracellular binding site (as defined herein) on said chimeric GPCR, said method comprising the steps of:
a) providing a composition that comprises (i) a chimeric GPCR, which chimeric GPCR comprises (at least) the extracellular loops of a first GPCR and the intracellular loops of a second GPCR (different from said first GPCR); and (ii) a binding domain or binding unit that can bind to a binding site on said chimeric GPCR that comprises at least one of said intracellular loops; and
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR.

The invention also relates to a method of identifying and/or generating compounds or ligands that are capable of binding to a functional conformation of a GPCR, said method comprising the steps of
a) providing a composition that comprises (i) a chimeric GPCR, which chimeric GPCR comprises (at least) the extracellular loops of said GPCR and the intracellular loops of a second GPCR (different from said first GPCR); and (ii) a binding domain or binding unit that can bind to a binding site on said chimeric GPCR that comprises at least one of said intracellular loops, in which said binding domain or binding unit is a conformation-inducing (as defined herein) binding domain or binding unit;
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR;
c) evaluating whether each of the test compounds or ligands (and/or which of said test compounds or ligands) binds to the chimeric GPCR; in said composition; and optionally
d) selecting the test compounds or ligands that bind to the chimeric GPCR in said composition.

The invention also relates to a method of identifying and/or generating compounds or ligands that are capable of binding to an active conformation of a GPCR, said method comprising the steps of:
a) providing a composition that comprises (i) a chimeric GPCR, which chimeric GPCR comprises (at least) the extracellular loops of said GPCR and the intracellular loops of a second GPCR (different from said first GPCR); and (ii) a binding domain or binding unit that can bind to a binding site on said chimeric GPCR that comprises at least one of said intracellular loops, in which said binding domain or binding unit is a conformation-inducing (as defined herein) binding domain or binding unit;
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR;
c) evaluating whether each of the test compounds or ligands (and/or which of said test compounds or ligands) binds to the chimeric GPCR; in said composition; and optionally
d) selecting the test compounds or ligands that bind to the chimeric GPCR in said composition.

The invention also relates to a method of identifying and/or generating compounds or ligands that are capable of binding to a functional conformation of a GPCR, said method comprising the steps of:
a) providing a composition that comprises (i) a chimeric GPCR that comprises the extracellular binding site (as defined herein) of said GPCR and that comprises the intracellular loops of another GPCR; and (ii) a binding domain or binding unit that can bind to a binding site on said chimeric GPCR that comprises at least one of said intracellular loops, in which said binding domain or binding unit is a conformation-inducing (as defined herein) binding domain or binding unit;
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR;
c) evaluating whether each of the test compounds or ligands (and/or which of said test compounds or ligands) binds to the chimeric GPCR; in said composition; and optionally
d) selecting the test compounds or ligands that bind to the chimeric GPCR in said composition.

The invention also relates to a method of identifying and/or generating compounds or ligands that are capable of binding to an active conformation of a GPCR, said method comprising the steps of:
a) providing a composition that comprises (i) a chimeric GPCR that comprises the extracellular binding site (as defined herein) of said GPCR and that comprises the intracellular loops of another GPCR; and (ii) a binding domain or binding unit that can bind to a binding site on said chimeric GPCR that comprises at least one of said intracellular loops, in which said binding domain or binding unit is a conformation-inducing (as defined herein) binding domain or binding unit;
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR;
c) evaluating whether each of the test compounds or ligands (and/or which of said test compounds or ligands) binds to the chimeric GPCR; in said composition; and optionally
d) selecting the test compounds or ligands that bind to the chimeric GPCR in said composition.

The invention also relates to a method of identifying and/or generating compounds or ligands that are capable of binding to a functional conformation of a GPCR, said method comprising the steps of:
a) providing a composition that comprises (i) a chimeric GPCR that comprises the extracellular loops of said GPCR and the TMs of said GPCR (or essentially all of the TMs of said GPCR, as further described herein) and that comprises the intracellular loops of another GPCR; and (ii) a binding domain or binding unit that can bind to a binding site on said chimeric GPCR that comprises at least one of said intracellular loops, in which said binding domain or binding unit is a conformation-inducing (as defined herein) binding domain or binding unit;
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR;
c) evaluating whether each of the test compounds or ligands (and/or which of said test compounds or ligands) binds to the chimeric GPCR; in said composition; and optionally
d) selecting the test compounds or ligands that bind to the chimeric GPCR in said composition.

The invention also relates to a method of identifying and/or generating compounds or ligands that are capable of binding to an active conformation of a GPCR, said method comprising the steps of:
a) providing a composition that comprises (i) a chimeric GPCR that comprises the extracellular loops of said GPCR and the TMs of said GPCR (or essentially all of the TMs of said GPCR, as further described herein) and that comprises the intracellular loops of another GPCR; and (ii) a binding domain or binding unit that can bind to a binding site on said chimeric GPCR that comprises at least one of said intracellular loops, in which said binding domain or binding unit is a conformation-inducing (as defined herein) binding domain or binding unit;
b) contacting said composition with one or more test compounds or ligands under conditions that (i) allow said binding domain or binding unit to bind to said binding site on the chimeric GPCR that comprises at least one of said intracellular loops; and (ii) allow said test compounds to bind to the extracellular binding site of said chimeric GPCR;
c) evaluating whether each of the test compounds or ligands (and/or which of said test compounds or ligands) binds to the chimeric GPCR; in said composition; and optionally
d) selecting the test compounds or ligands that bind to the chimeric GPCR in said composition.

Again, in all these methods, the chimeric GPCR and the binding domain or binding unit that are present in the compositions used are preferably again as further described herein (with any preferences or preferred aspects described herein for a chimeric GPCR of the invention and/or for such a binding domain/binding unit also being preferred for use in said method). Also, again, the chimeric GPCR that is present in the compositions used, and the conditions under which the composition is used in the above steps are preferably chosen such, that binding of the test compounds to the chimeric GPCR under the conditions used is representative for binding of said test compound(s) to the GPCR from which the extracellular binding site has been derived. The chimeric GPCR or the binding domain or binding unit may also be immobilized on a solid support, again as further described herein. The chimeric GPCR or the binding domain or binding unit may also be suitably provided and used as a fusion protein, as further described herein and in the International application WO 2014/118297.

Also, in methods in which a composition is used that comprises a chimeric GPCR of the invention and a binding domain or binding unit that can bind to an intracellular binding site of said chimeric GPCR, said composition can be a cellular composition as described herein, or the composition may comprise in a suitable liposome or vesicle that suitably contains (as described herein) the chimeric GPCR and the binding domain or binding unit.

In further aspects, the invention also relates to:
a composition that comprises (i) a chimeric GPCR, which chimeric GPCR comprises (at least) the extracellular loops of a first GPCR and the intracellular loops of a second GPCR (different from said first GPCR); and (ii) a binding domain or binding unit that can bind to a binding site on said chimeric GPCR that comprises at least one of said intracellular loops, in which said binding domain or binding unit is preferably a conformation-inducing (as defined herein) binding domain or binding unit;
a composition that comprises (i) a chimeric GPCR, which chimeric GPCR comprises the extracellular binding site (as defined herein) of a first GPCR and the intracellular loops of a second GPCR (different from said first GPCR); and (ii) a binding domain or binding unit that can bind to a binding site on said chimeric GPCR that comprises at least one of said intracellular loops, in which said binding domain or binding unit is preferably a conformation-inducing (as defined herein) binding domain or binding unit;
a composition that comprises (i) a chimeric GPCR that comprises the extracellular loops of a first GPCR and the TMs of said first GPCR (or essentially all of the TMs of said first GPCR, as further described herein) and that comprises the intracellular loops of a second GPCR (different from said first GPCR); and (ii) a binding domain or binding unit that can bind to a binding site on said chimeric GPCR that comprises at least one of said intracellular loops, in which said binding domain or binding unit is preferably a conformation-inducing (as defined herein) binding domain or binding unit.

The invention also relates to uses of such compositions, in particular in methods as described herein.

Said compositions can be a cellular composition as described herein, or the composition may comprise in a suitable liposome or vesicle that suitably contains (as described herein) the chimeric GPCR and the binding domain or binding unit. Also, the chimeric GPCR and the binding domain or binding unit that are present in said compositions used are preferably again as further described herein (with any preferences or preferred aspects described herein for a chimeric GPCR of the invention and/or for such a binding domain/binding unit also being preferred for use in said method). Furthermore, the chimeric GPCR or the binding domain or binding unit may also be immobilized on a solid support, again as further described herein. The chimeric GPCR or the binding domain or binding unit may also be suitably provided and used as a fusion protein, as further described herein and in the International application WO 2014/118297.

As described herein, in some preferred aspects of the invention, the chimeric GPCR of the invention is present in and/or expressed by a suitable cell or cell line.

Thus, in a further aspect, the invention relates to a cell or cell line that comprises, expresses and/or is capable of expressing a chimeric GPCR of the invention. Such a cell or cell line is preferably such that said chimeric GPCR is present in (i.e. anchored in) the cell membrane or cell wall of said cell or cell line and/or such that said cell or cell line suitably expresses (as defined herein) said chimeric GPCR. More preferably, said cell or cell line is such (and/or is capable of expressing the chimeric GPCR such) that the chimeric GPCR of the invention spans the cell membrane or cell wall of said cell or cell line such that the extracellular loops extend out into the extracellular environment and the intracellular loops extend out into the intracellular environment of said cell or cell line. In the context of the present application and claims, when one or more ECLs are said to “extend out” into an environment (such as an extracellular environment of a cell or the environment outside of a liposome or vesicle), this should generally be understood to mean that said ECLs are exposed to said environment and/or is accessible for binding by a ligand, compound or other chemical entity that is present in said environment. In particular, for a chimeric GPCR of the invention, this means that the extracellular binding site (as defined herein) of the chimeric protein is accessible for binding by a ligand, compound or other chemical entity that is present in said environment. Similarly, when one or more ICLs are said to “extend out” into an environment (such as the intracellular environment of a cell or the environment insider of a liposome or vesicle), this should generally be understood to mean that said ICLs are exposed to said environment and/or is accessible for binding by a ligand, compound or other chemical entity that is present in said environment. In this respect, it should also be noted that the wording “accessible for binding” should generally be taken to mean that a ligand, compound or other chemical entity that is present in the relevant environment can bind to a binding pocket or binding site on or within the chimeric GPCR, even if the actual binding site or binding pocket lies deep(er) within the structure of the chimeric GPCR (even such that the actual binding site or binding pocket is located within a part of the chimeric that itself does not physically extend out beyond the boundary layer). Reference is for example made to the publication by Chevillard (cited herein) which shows that the binding sites on GPCRs for fragments that are used in FBDD screening techniques may lie deep within the GPCR structure (see for example FIG. 2 on page 1120) and not be on the surface of the GPCR, but nevertheless are accessible for fragment binding. Reference is also made to the teachings on GPCR structure, GPCR signaling mechanisms and GPCR ligand binding sites from some of the other scientific references cited herein.

A cell or cell line that comprises or expresses a chimeric GPCR of the invention is preferably further such that it also comprises expresses and/or is capable of expressing a binding domain or binding unit that can specifically bind to (the binding site formed by) the intracellular loops that are present in said chimeric protein. Again, such a binding domain or binding unit that is present in and/or expressed by said cell or cell line preferably a conformation-inducing (as defined herein) binding domain or binding unit and more preferably a conformation-inducing ISVD (such as a ConfoBody). Also, said cell or cell line is preferably such that it contains or expresses said binding domain or binding unit such that it can bind to the intracellular binding site (as defined herein) of the chimeric GPCR of the invention (which, as will be clear to the skilled person, will usually mean that said cell or cell line contains or expresses said binding domain or binding unit within its intracellular environment).

Such a cell or cell line that that comprises or expresses a chimeric GPCR of the invention (and preferably also a binding domain or binding unit that can specifically bind to the intracellular loops that are present in said chimeric protein) can generally be as further described herein.

The invention also relates to uses of a cell or cell line that contains, expresses and/or is capable of expressing a chimeric GPCR of the invention. In particular, the invention relates to uses of a cell or cell line that contains, expresses and/or is capable of expressing a chimeric GPCR of the invention in:
identifying, generating and/or screening for ligands or compounds that can bind (and in particular, specifically binding as defined herein) to the chimeric GPCR that is present in said complex (e.g. using standard screening techniques known per se); and/or
determining at least one property of a compound or ligand, such as its ability to bind (and in particular, specifically bind as defined herein) to the chimeric GPCR that is present in said complex and/or to modulate said chimeric GPCR (e.g. using standard assay techniques known per se).

For such uses, the cell or cell line that contains, expresses and/or is capable of expressing a chimeric GPCR of the invention can be as further described herein. Preferably, the cell or cell line used is also such that it contains, expresses or is capable of expressing a binding domain or binding unit that can specifically bind to (the binding site formed by) the intracellular loops that are present in said chimeric protein. Such a binding domain is again preferably a conformation-inducing binding domain or binding unit (as defined herein) and again preferably an ISVD (and preferably a conformation-inducing ISVD).

The invention also relates to a method for determining at least one property of a compound or ligand, said method comprising at least the steps of.
providing a cell or cell line that contains, expresses or is capable of expressing of a chimeric GPCR of the invention, which cell or cell line preferably also contains, expresses or is capable of expressing a binding domain or binding unit that can specifically bind to (the binding site formed by) the intracellular loops that are present in said chimeric GPCR, which binding domain or binding unit is preferably a conformation-inducing (as defined herein) binding domain or binding unit;
contacting said cell or cell line with said compound or ligand, in which said compound or ligand is present in the extracellular environment and in which said cell or cell line is such that the extracellular binding site (as defined herein) on the chimeric GPCR is available/accessible for binding by a compound or ligand that is present in the extracellular environment.

Where required, said method may also comprise a step of maintaining or cultivating said cell or cell line under conditions such that said cell or cell line expresses (and in particular suitably expresses, as defined herein) the chimeric GPCR of the invention and also expresses said binding domain or binding unit such that said binding domain or binding unit can bind to the ICLs of the chimeric GPCR and/or form a complex (as described herein) with said chimeric GPCR and optionally also with said compound or ligand.

Said method again preferably also comprises the step of measuring (the change in) at least one signal or parameter that is representative for said at least one property. As described herein, said property can be the ability to bind (and in particular the ability to specifically bind) to the chimeric GPCR of the invention. Said at least one property can also be the ability to modulate said chimeric GPCR, for example the ability to act as an agonist (e.g. a partial or a full agonist) of the chimeric GPCR, the ability to act as an antagonist of the chimeric GPCR as an antagonist, and/or the ability to act as an inverse agonist of the chimeric GPCR. Again, preferably, said at least one property as determined using a chimeric GPCR of the invention is representative for essentially the same or an essentially similar property of the naturally occurring GPCR from which the ECLs (and preferably essentially also the TMs, as further described herein) have been derived.

The invention also relates to a method for forming a complex that comprises:
a) a chimeric protein comprising an N-terminal sequence, a C-terminal sequence, seven transmembrane domains (TM1 to TM7), three extracellular loops (EC1 to EC3) and three intracellular loops (IC1 to IC3), in which:
   the extracellular loops that are present in the chimeric protein are (essentially) derived from a first GPCR; and
   the intracellular loops that are present in the chimeric protein are (essentially) derived from a second GPCR;
      and:
b) a binding domain or binding unit that can specifically bind to (the binding site formed by) the intracellular loops that are present in said chimeric protein;
   and optionally:
c) a ligand or compound that is bound to the extracellular binding site (as defined herein) on the chimeric GPCR;
   which method at least comprises the step of:
   maintaining or cultivating a cell or cell line that expresses a chimeric protein as referred to under a) and a binding domain or binding unit as referred to under b) under conditions such that said cell or cell line expresses said chimeric protein and said binding domain; and optionally also comprises the step of
   contacting said cell or cell line with the ligand or compound as referred to under c).

Again, said cell or cell line is preferably such that the extracellular loops of the chimeric GPCR extend out (as defined herein) into the extracellular environment and/or such that the extracellular binding site on the chimeric GPCR is available/accessible for binding by the ligand or compound as referred to under c) when said ligand or compound is present in the extracellular environment. Also, again, the binding domain or binding unit that is present in and/or expressed by said cell or cell line preferably a conformation-inducing (as defined herein) binding domain or binding unit and more preferably a conformation-inducing ISVD (such as a ConfoBody). Also, preferably, the chimeric GPCR in said complex (once it is formed) is preferably in a functional, active and/or druggable conformation. In one specific aspect, the ligand or compound referred to under c) is an agonist and the chimeric GPCR in said complex (once it is formed) is in an agonist-bound conformation.

As described herein, it is envisaged in the invention that a chimeric GPCR of the invention may be fused to a binding domain or binding unit that can specifically bind to (the binding site formed by) the intracellular loops that are present in said chimeric protein. Such fusions and their uses may essentially be as described in the International application WO 2014/118297 entitled “Novel chimeric polypeptides for screening and drug discovery purposes”, which describes fusions of GPCRs and ConfoBodies that are specific for the GPCR and in particular for an intracellular binding site on the GPCR.

Generally, such a fusion protein will comprise a chimeric GPCR of the invention as described herein that is fused or linked, optionally via a suitable spacer or linker, to a binding domain or binding unit that can specifically bind to (the binding site formed by) the intracellular loops that are present in said chimeric protein. The spacer or linker that is present in such a fusion protein may also essentially be as described in the International application WO 2014/118297. Also, the chimeric GPCR and the binding unit or binding domain that is present in said fusion protein may essentially be as further described herein. In particular, the binding unit or binding domain may be a conformation-inducing binding domain or binding unit and more preferably a conformation-inducing ISVD (such as a ConfoBody).

The invention also relates to uses of such fusion proteins, in particular for assay drug discovery and screening purposes. Such uses may be as further described herein and/or in the International application WO 2014/118297. For such uses, the chimeric protein may be expressed in a suitable cell or cell line (essentially as described herein and in the International application WO 2014/118297) and cells or cell lines that express such fusion proteins form further aspects of the invention. Also, for such uses, such a fusion protein may be immobilized on a solid support (again, essentially as described herein and in the International application WO 2014/118297) and such a fusion protein that is immobilized on a solid support and a solid support upon which is immobilized such a fusion protein form further aspects of the invention.

Thus, in a further aspect, the invention relates to a method of identifying and/or generating compounds or ligands that are capable of binding to an extracellular binding site on a GPCR, said method comprising the steps of:
a) providing a fusion protein comprising: (i) a chimeric GPCR that essentially comprises (at least) the extracellular binding site of said GPCR and that comprises the intracellular loops of another GPCR, which chimeric GPCR is fused or linked, optionally via a suitable spacer or linker to (ii) a binding domain or binding unit that can bind to a binding site on said chimeric GPCR that comprises at least one of said intracellular loops;
b) contacting said fusion protein with one or more test compounds or ligands under conditions that allow said test compounds to bind to the extracellular binding site of the chimeric GPCR that is present in said fusion protein;
c) evaluating whether each of the test compounds or ligands (and/or which of said test compounds or ligands) binds to the chimeric GPCR that is present in said fusion protein; and optionally
d) selecting the test compounds or ligands that bind to the chimeric GPCR that is present in said fusion protein.

In another aspect, the invention relates to a method of identifying and/or generating compounds or ligands that are capable of binding to a GPCR, said method comprising the steps of:
a) providing a fusion protein comprising: (i) a chimeric GPCR that comprises the extracellular loops of said GPCR and the TMs of said GPCR (or essentially all of the TMs of said GPCR, as further described herein) and that comprises the intracellular loops of another GPCR, which chimeric GPCR is fused or linked, optionally via a suitable spacer or linker to (ii) a binding domain or binding unit that can bind to a binding site on said chimeric GPCR that comprises at least one of said intracellular loops;
b) contacting said fusion protein with one or more test compounds or ligands under conditions that allow said test compounds to bind to the extracellular binding site of the chimeric GPCR that is present in said fusion protein;
c) evaluating whether each of the test compounds or ligands (and/or which of said test compounds or ligands) binds to the chimeric GPCR that is present in said fusion protein; and optionally
d) selecting the test compounds or ligands that bind to the chimeric GPCR that is present in said fusion protein.

In the methods of the invention in which such a fusion protein is used, the chimeric GPCR and the binding domain or binding unit that are present in said fusion protein are again as further described herein. Also, again, the chimeric GPCR that is present in said fusion protein, and the conditions under which said fusion protein and the binding domain or binding unit are used in the above steps are preferably chosen such, that binding of the test compounds to the chimeric GPCR under the conditions used is representative for binding of said test compound(s) to said GPCR. Also, in such a method, the fusion protein may be present in a suitable cellular composition and/or expressed by a suitable cell or cell line or they may be present in a suitable liposome or vesicle, all as further described herein. The fusion protein may also be immobilized on a solid support, again as further described herein.

Also, as with the other methods described herein, methods involving the use of such a fusion protein may be used to identify and/or generate compounds or ligands that bind to the extracellular binding site on the chimeric GPCR that is present is said fusion protein and/or compounds or ligands that bind to an allosteric site on the chimeric GPCR.

In a further aspect, the invention relates to a method of identifying and/or generating compounds or ligands that are capable of binding to an extracellular binding site of a GPCR, said method comprising the steps of:
a) providing a cell or cell line that contains a fusion protein comprising: (i) a chimeric GPCR that essentially comprises (at least) the extracellular binding site of said GPCR and that comprises the intracellular loops of another GPCR, which chimeric GPCR is fused or linked, optionally via a suitable spacer or linker to (ii) a binding domain or binding unit that can bind to a binding site on said chimeric GPCR that comprises at least one of said intracellular loops;
b) contacting said cell or cell line with one or more test compounds or ligands under conditions that allow said test compounds to bind to (at least) the extracellular loops of said chimeric GPCR;
c) evaluating whether each of the test compounds or ligands (and/or which of said test compounds or ligands) binds to said chimeric GPCR that is present in said cell or cell line; and optionally
d) selecting the test compounds or ligands that bind to said chimeric GPCR.

In this method of the invention, the cell or cell line that contains said fusion protein can in particular be provided by maintaining or culturing a cell or cell line that is capable of expressing said fusion protein under conditions such that said cell line expresses said fusion protein, preferably such that the chimeric GPCR in the fusion protein becomes anchored or incorporated in the cell wall or cell membrane of said cell or cell line (as generally described herein for the chimeric GPCRs of the invention) and such that the binding domain or binding unit that is part of the fusion protein is expressed in the intracellular environment such that it can bind to the intracellular binding site (as defined herein) on the chimeric protein.

In a further aspect, the invention relates to a method of identifying and/or generating compounds or ligands that are capable of binding to an extracellular binding site of a GPCR, said method comprising the steps of:
a) providing a liposome or vesicle that contains a fusion protein comprising: (i) a chimeric GPCR that essentially comprises (at least) the extracellular binding site of said GPCR and that comprises the intracellular loops of another GPCR, which chimeric GPCR is fused or linked, optionally via a suitable spacer or linker to (ii) a binding domain or binding unit that can bind to a binding site on said chimeric GPCR that comprises at least one of said intracellular loops;
b) contacting said cell or cell line with one or more test compounds or ligands under conditions that allow said test compounds to bind to (at least) the extracellular loops of said chimeric GPCR;
c) evaluating whether each of the test compounds or ligands (and/or which of said test compounds or ligands) binds to said chimeric GPCR that is present in said cell or cell line; and optionally
d) selecting the test compounds or ligands that bind to said chimeric GPCR.

In further aspects, the invention also relates to compositions that comprise such fusion proteins, and to uses of such fusion proteins and such compositions, in particular in the methods described herein. Again, such compositions can be cellular compositions as described herein or comprise vesicles or liposomes that suitably contain such a fusion protein, as further described herein.

It is also envisaged in the invention that a chimeric GPCR of the invention can find use in methods and arrangements that are as described in the co-pending US provisional application filed on Apr. 29, 2019 and entitled “Screening methods and assays for use with transmembrane proteins, in particular with GPCRs” (herein also referred to as the “Co-Pending Application”), which co-pending application has been assigned to Confo Therapeutics N.V., the disclosure of which is incorporated herein by reference.

The Co-Pending Application generally describes an arrangement that comprises at least the following elements (all as further defined in the Co-Pending Application):
a boundary layer that separates a first environment from a second environment;
a translayer protein;
a first ligand for the translayer protein that is present in (as defined herein) the first environment;
a second ligand for the translayer protein that is present in (as defined herein) the second environment; and
a binding pair that consists of at least a first binding member and a second binding member, which binding pair is capable of generating a detectable signal;
   and also describes uses of said arrangements (in particular for assay and screening techniques) and methods using such arrangements (which again, can in particular be assays and screening methods).

In one specific and preferred aspect, the chimeric proteins of the invention are used as the translayer protein in the arrangements and methods described in the Co-Pending Application.

Generally, this means that such an arrangement will comprise, as the translayer protein, a chimeric GPCR in which the intracellular loops are be derived from a first 7TM or GPCR and the extracellular loops are be derived from a second 7TM or GPCR different from the first. The transmembrane domains of such a chimeric protein may be derived from the first or the second 7TM or GPCR, and are preferably essentially all derived from the same GPCR, and are more preferably derived from the same GPCR as the extracellular loops (but may contain some amino acid residues from the GPCR from which the intracellular loops have been derived, depending on the positions chosen for recombinantly deleting the native intracellular loops and inserting the replacement intracellular loops).

In this aspect of the invention, the resulting chimeric translayer protein should most preferably still be such that it can be suitably used in the methods and arrangements as described in the Co-Pending Application. Again, the chimeric GPCR of the invention that is used as the translayer protein will comprise three intracellular loops and three extracellular loops, with the three intracellular loops forming a functional ligand binding site for the second ligand (which second ligand will then be selected such that it can bind to the ligand binding site (9) that is formed by said intracellular loops). Again, the binding site that is formed by the three intracellular loops will preferably extend out into the second environment [B] (i.e. the environment inside of the cell or liposome when the methods of the invention are performed in cells or liposomes, respectively) and the three extracellular loops will preferably extend out into the first environment [A] (and may form a functional binding site for the first ligand or said binding site may lie deeper within the structure of the 7TM).

Thus, in a further aspect, the invention relates to an arrangement as described in the Co-Pending Application, in which the translayer protein is a 7TM that comprises 7 transmembrane domains, 3 intracellular loops and 3 extracellular loops (which are linked to each other and in an order as is known per se for 7TMs, i.e. [N-terminal sequence]-[TM1]-[IC1]-[TM2]-[EC1]-[TM3]-[IC2]-[TM4]-[EC2]-[TM5]-[IC3]-[TM6]-[EC3]-[TM7]-C-terminal sequence), in which the intracellular loops are derived from a first 7TM and the extracellular loops are derived from a second 7TM different from the first 7TM, in which the intracellular loops form a functional ligand binding site. Preferably, the TM domains from said translayer protein are essentially derived from the same 7TM as the extracellular loops.

Also, said intracellular loops and the 7TM as a whole are such that they form a functional ligand binding site, and in particular a functional ligand binding site to which a (suitable) second ligand (as defined herein) can bind. Said ligand binding site again preferably extends out into the second environment [B].

The invention in particular relates to an arrangement as described in the Co-Pending Application that comprises such a chimeric 7TM and a second ligand that can bind to the ligand binding site that is formed by said intracellular loops.

For the remainder, provided that the second ligand is suitably chosen such that it can bind to the ligand binding site (9) on the chimeric translayer protein so as to provide an operable arrangement as described in the Co-Pending Application (and provided that the chimeric translayer protein itself is operable in such an arrangement), such arrangements in which a chimeric translayer protein is used can be essentially as further described herein and in the Co-Pending Application.

Another aspect of the invention is a composition or kit-of-parts as described in the Co-Pending Application that comprises at least said chimeric translayer protein and a ligand that can bind to the intracellular loops that are present in said GPCRs. Said ligand is preferably a protein and more preferably a protein that comprises or essentially consists of an immunoglobulin single variable domain (such as a VHH domain) and may in particular be a ConfoBody (as described herein).

It should also be understood that, when in the further description herein and in the claims reference is made to such an arrangement or to any element of such an arrangement, such arrangement or element(s) are generally (and preferably) as further described in the Co-Pending Application. Also, any terms not specifically defined otherwise herein should generally be understood as having the meaning set forth in the Co-Pending Application.

Thus, in a further aspect, the invention relates to an arrangement that comprises at least the following elements (all as further defined herein):
a boundary layer that separates a first environment from a second environment;
a translayer protein;
a first ligand for the translayer protein that is present in (as defined herein) the first environment;
a second ligand for the translayer protein that is present in (as defined herein) the second environment; and
a binding pair that consists of at least a first binding member and a second binding member, which binding pair is capable of generating a detectable signal;
   in which said translayer protein is a chimeric GPCR of the invention as described herein and in which the elements of the arrangement are arranged with respect to each other (and where applicable operably linked to and/or associated with each other) in the manner as further described herein and in the Co-Pending Application.

In a specific aspect of such an arrangement, the second ligand will be a binding domain or binding unit as described herein, i.e. a binding domain or binding unit that can specifically bind to (the binding site formed by) the intracellular loops that are present in said chimeric protein.

As also described in the Co-Pending Application, the arrangements and methods of the Co-Pending Application generally (and preferably) comprise the use of two fusion proteins, namely a first fusion protein that comprises the translayer protein and the first binding member of the binding pair, and a second fusion protein comprising the second member of the binding pair and a protein that can bind directly or indirectly (as defined in the Co-Pending Application) to the translayer protein. Accordingly, when a chimeric protein of the invention is used as the translayer protein in such methods and arrangements, it may be part of such a first fusion protein, together with a first binding member of the binding pair used in such an arrangement.

Thus, in a further aspect, the invention relates to a fusion protein that comprises a chimeric protein of the invention which is linked, via a suitable linker or spacer, to a binding domain, binding unit or other peptide, protein or amino acid sequence that is a member of a binding pair, which binding pair is as further described herein and in the Co-Pending Application. The invention also relates to nucleotide sequences and/or nucleic acids that encode such a fusion protein and to cells, cell lines or other host cells or host organisms that express (and in particularly suitably express, as described in the Co-Pending Application) or are capable of (suitably) expressing such a fusion protein.

In particular, an arrangement for performing the methods of the invention may comprise at least the following elements:
a boundary layer that separates a first environment from a second environment;
a binding pair that consists of at least a first binding member and a second binding member, which binding pair is capable of generating a detectable signal;
a translayer protein that is suitably fused or linked (either directly or via a suitable linker or spacer) to one of the binding members of said binding pair (i.e. so as to form a first fusion protein);
a first ligand for the translayer protein that is present in the first environment; and
a second ligand for the translayer protein that is present in the second environment; in which said translayer protein is a chimeric GPCR of the invention as described herein and in which the elements of the arrangement are arranged with respect to each other (and where applicable operably linked to and/or associated with each other) in the manner as further described herein. As further described herein and in the Co-Pending Application, the second member of the binding pair may be part of a second fusion protein (which is different from the first fusion protein that comprises the translayer protein and the first binding member of the binding pair), which second fusion protein is as further described herein.

More in particular, an arrangement for performing the methods of the invention may comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a second fusion protein comprising a protein that can bind directly or indirectly to the translayer protein and the other binding member of said binding pair, which second fusion protein is present in the second environment; and
a first ligand for the translayer protein that is present in the first environment; in which said translayer protein is a chimeric GPCR of the invention as described herein and in which the elements of the arrangement are arranged with respect to each other (and where applicable operably linked to and/or associated with each other) in the manner as further described herein.

It should be noted that in the present description and claims, when it is said that a ligand, binding domain, binding unit or other compound or protein “can bind to” another protein or compound, that such binding is most preferably “specific binding” as further defined herein. Also, as further described herein, when a fusion protein is described as “comprising” a first protein, ligand, binding domain, binding member or binding unit and a second protein, ligand, binding domain, binding member or binding unit (and optionally one or more further proteins, ligands, binding domains, binding members or binding units), it should be understood that in such a fusion protein, such proteins, ligands, binding domains, binding members or binding units are suitably linked to each other, either directly or via a suitable spacer or linker.

As generally described in the Co-Pending Application, in an arrangement according to the Co-Pending application, a protein (such as a binding domain, binding unit or ligand) is said to bind “directly or indirectly” to the translayer protein if (i) said protein itself binds (and/or is capable of binding) to the translayer protein (e.g. to an epitope or binding site on the translayer protein, as further described herein); or if (ii) said protein binds (and/or is capable of binding) to a ligand or protein that binds (and/or is capable of binding) to said translayer protein; or if (iii) said protein binds (and/or is capable of binding) to a protein complex that comprises a ligand or protein that binds (and/or is capable of binding) to said translayer protein. In the case of (i), the protein is said herein to bind “directly” to the translayer protein, and in the case of (ii) and (iii), the protein is said herein to bind “indirectly” to the translayer protein. Also, when a protein binds to a protein complex that comprises a ligand or protein that binds to the translayer protein, said protein may bind to said ligand or protein or to any other part, epitope or binding site of said complex).

When a chimeric GPCR of the invention is used as the translayer protein in an arrangement according to the Co-Pending Application, the protein that binds to the translayer protein (i.e. to the chimeric GPCR of the invention) can also be chosen from (i) a binding domain, binding unit or other protein that binds (and/or is capable of binding) to an epitope or binding site on the translayer protein; (ii) a binding domain, binding unit or other protein that binds (and/or is capable of binding) to a ligand or protein that binds (and/or is capable of binding) to said translayer protein; and (iii) a binding domain, binding unit or other protein that binds (and/or is capable of binding) to a protein complex that comprises a ligand or protein that binds (and/or is capable of binding) to said translayer protein. In each such case, such a binding domain, binding unit or other protein is preferably as further described herein.

When, in such an arrangement comprising a chimeric GPCR of the invention, the protein that binds to the translayer protein (i.e. to the chimeric GPCR of the invention) is a protein that binds “indirectly” to the chimeric GPCR of the invention, then said protein and the second ligand will be as further described in the Co-Pending Application and the arrangement will usually not comprise a binding domain or binding unit as described herein (i.e. a conformation-inducing binding domain or binding unit that can bind to the intracellular loops of the chimeric GPCR).

However, when in such an arrangement comprising a chimeric GPCR of the invention, the protein that binds to the translayer protein (i.e. to the chimeric GPCR of the invention) is a protein that binds “directly” to the chimeric GPCR of the invention, then said arrangement will comprise a binding domain or binding unit as described herein, which binding domain or binding unit will serve as the “second ligand”. Also, as generally described in the Co-Pending Application, when said protein binds “directly” to the translayer protein, it is preferably part of the second fusion protein. Accordingly, when such an arrangement that comprises a chimeric GPCR of the invention also comprises a binding domain or binding unit as described herein as the second ligand, then said binding domain or binding unit most preferably also forms part of the second fusion protein.

Thus, in a further aspect, the invention relates to a fusion protein that comprises a chimeric protein of the invention which is linked, via a suitable linker or spacer, to a binding domain, binding unit or other peptide, protein or amino acid sequence that is a member of a binding pair, which binding pair is as further described herein and in the Co-Pending Application. The invention also relates to nucleotide sequences and/or nucleic acids that encode such a fusion protein and to cells, cell lines or other host cells or host organisms that express (and in particularly suitably express, as described in the Co-Pending Application) or are capable of (suitably) expressing such a fusion protein.

In a further aspect of the invention, an arrangement for performing the methods of the invention may comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a second fusion protein comprising a protein that can bind directly (as defined herein) to the translayer protein and the other binding member of said binding pair, which second fusion protein is present in the second environment; and
a first ligand for the translayer protein that is present in the first environment; in which said translayer protein is a chimeric GPCR of the invention as described herein and in which said protein is a binding domain or binding unit that can bind to a binding site on said chimeric GPCR that comprises at least one of said intracellular loops (and is preferably a conformation-inducing binding domain as described herein) and in which the further elements of the arrangement are arranged with respect to each other (and where applicable operably linked to and/or associated with each other) in the manner as further described herein. In this aspect of the invention, the protein that can bind directly (as defined herein) to the translayer protein (i.e. to the chimeric GPCR of the invention) and that is present in the second fusion protein is preferably a binding domain or binding unit and more preferably an immunoglobulin single variable domain. It should also be understood that in this aspect of the invention, the protein that can bind directly (as defined herein) to the translayer protein and that is present in the second fusion protein acts as the second ligand.

In another aspect of the invention, an arrangement for performing the methods of the invention may comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment;
a second ligand for the translayer protein, which may optionally be part of a protein complex;
a second fusion protein comprising a protein that can bind indirectly (as defined herein) to the translayer protein and the other binding member of said binding pair, which second fusion protein is present in the second environment;
   in which said translayer protein is a chimeric GPCR of the invention as described herein and in which the elements of the arrangement are arranged are arranged with respect to each other (and where applicable operably linked to and/or associated with each other) in the manner as further described herein. In this aspect of the invention, the second ligand can be any suitable ligand (as further described herein) but is most preferably a G-protein (or G-protein complex) and the protein that can bind indirectly (as defined herein) to the translayer protein and that is present in the second fusion protein is preferably a binding domain or binding unit and more preferably an immunoglobulin single variable domain. Also, when the second ligand is a G-protein or G-protein complex, the chimeric GPCR of the invention is most preferably such that its ICLs form (or form part of) a functional binding site for a G-protein. It will also be clear that in this aspect of the invention, the second ligand will not form part of the second fusion protein.

It should be noted that, as further described herein, in the practice of the invention, the first ligand will often be added to the further elements of an already formed/established arrangement of the invention as described herein, and that consequently arrangements of the invention without the first ligand being present (i.e. before the first ligand is added) form further aspects of the invention (as do methods in which a first ligand is added to an arrangement of the invention in which said first ligand is not or not yet present).

In the present description and claims, the term “second ligand” is used to denote the ligand, binding domain, binding unit or other chemical entity that, in the methods and arrangements described herein, binds directly to the translayer protein (i.e. to the chimeric GPCR of the invention) or is capable of binding directly to the translayer protein (or forms part of a protein complex that binds directly to the translayer protein or that is capable of binding directly to the translayer protein).

As will be clear from the further description herein, when the chimeric protein of the invention is used as part of an arrangement as described herein and in the Co-Pending Application, said second ligand can either be part of the second fusion protein or it can be separate from the second fusion protein. In either case (i.e. irrespective of whether the second ligand is part of the second fusion protein or not), the second ligand is preferably such that it is capable of binding to a conformational epitope on the chimeric GPCR (or such that it can form part of a protein complex that binds directly to the chimeric GPCR or that is capable of binding directly to the chimeric GPCR), and in particular to the conformational epitope on the chimeric GPCR that comprises one or more of the ICls. More preferably, the second ligand (and/or the protein complex that comprises the second ligand) is preferably such that it specifically binds to one or more functional, active and/or druggable conformations of the translayer protein (i.e. of the chimeric GPCR of the invention), that it induces the formation of and/or stabilizes one or more functional, active and/or druggable conformations of the translayer protein (and/or shifts the conformational equilibrium of the translayer protein towards one or more such conformations); and/or that it induces the formation of and/or stabilizes a complex of the translayer protein, the first ligand and the second ligand.

When the second ligand is part of the second fusion protein, it can be any ligand, binding domain, binding unit, peptide, protein or other chemical entity that can bind directly to the ICLs of the chimeric protein of the invention that is used as the translayer protein and that can suitably be included in the second fusion protein. Preferably, as further described herein, when it is part of the second fusion protein, the second ligand will be a suitable binding domain or binding unit, and in particular an immunoglobulin single variable domain (and preferably, as conformation-inducing ISVD as defined herein). Again, in this aspect of the invention, when a chimeric GPCR of the invention is used as the translayer protein together with an immunoglobulin single variable domain that is specific for the ICLs that are present in the chimeric GPCR (which ISVD is used as the “second ligand” that is present in the “second fusion protein”), this avoids any issues or limitations that may be associated with the need to provide the desired GPCR in an isolated and suitably purified form and in a desired conformation for screening and selection purposes and, when naïve libraries are to be used, for immunization and display purposes.

When the second ligand is separate from the second fusion protein, it can be any ligand or protein that can bind directly to the translayer protein (i.e. to the chimeric GPCR of the invention) and/or that can form part of a protein complex that can bind to the translayer protein, but it is preferably a G-protein or G-protein complex (in particular in those aspects of the invention in which the ICLs that are present in the chimeric GPCR of the invention form a functional binding site for a G-protein or G-protein complex). For example, as further described herein, in such aspects of the invention, the second ligand may be a naturally occurring G-protein, such as—when an arrangement as described herein that comprises a chimeric GPCR of the invention is present in a cell—the G-protein may be the G-protein that is natively expressed by the cell in which the chimeric GPCR of the invention is present or expressed. Such a second ligand may also be a semi-synthetic or synthetic analog or derivative of a naturally occurring ligand G-protein or, again when the arrangement of the invention is present in a cell, it may be an ortholog of the G-protein that naturally occurs in said cell. Also, when the second ligand is not part of the second fusion protein, the second fusion protein will comprise a binding domain or binding unit that can bind indirectly (as defined herein) to the translayer protein, i.e. a binding domain or binding unit that can bind to the second ligand and/or to a protein complex that comprises the second ligand. Again, as also further described herein, such a binding domain or binding unit may in particular be an immunoglobulin single variable domain, such as a camelid-derived ISVD.

As further described herein, in one aspect of the invention, an arrangement as described herein that comprises and/or uses a chimeric GPCR of the invention can be present in a suitable cell or cell line and/or the methods of the invention can be performed using a suitable cell or cell line that suitably expresses a chimeric GPCR of the invention and/or that contains a chimeric GPCR of the invention in an (operable) arrangement that is present in said cell or cell line. Such a cell or cell line can again be as further described herein, and can also express a second fusion protein that comprises a binding domain or binding unit that can bind to the ICls that are present in the chimeric GPCR.

In aspects of the invention where a chimeric GPCR of the invention is used as part of an arrangement as described herein and in the Co-Pending Application, such a cell or cell line most preferably also contains and/or suitably expresses (or is capable of suitably expressing) the further elements of such an arrangement, in particular so as to provide an arrangement that is operable in said cell or cell line. The invention also relates to a cell or cell line that comprises and/or that suitably expresses (as defined herein) or is capable of suitably expressing a first fusion protein as described herein, which first fusion protein comprises a chimeric GPCR of the invention. The invention also relates to a cell or cell line that comprises and/or that suitably expresses or is capable of suitably expressing a second fusion protein as described herein. In yet another aspect, the invention relates to a cell or cell line that comprises and/or that suitably expresses or is capable of suitably expressing both a first fusion protein comprising a chimeric GPCR of the invention as described herein and a second fusion protein as described herein. In aspects and embodiments where the second ligand does not form part of the second fusion protein, such cells or cell lines may also contain or suitably express a suitable second ligand, an in particular a G-protein or analog or derivative thereof, as further described herein.

As also described herein, in one aspect of the invention, an arrangement as described herein that comprises and/or uses a chimeric GPCR of the invention can be present in a suitable liposome or vesicle and/or the methods of the invention can be performed using a liposome or vesicle that suitably contains a chimeric GPCR of the invention in an (operable) arrangement as described herein. Such a vesicle or liposome can again be as further described herein, and can also contain second fusion protein that comprises a binding domain or binding unit that can bind to the ICls that are present in the chimeric GPCR.

In aspects of the invention where a chimeric GPCR of the invention is used as part of an arrangement as described herein and in the Co-Pending Application, such a liposome or vesicle most preferably also contains the further elements of such an arrangement, in particular so as to provide an arrangement that is operable in said liposome or vesicle. The invention also relates to a liposome or vesicle that comprises a first fusion protein as described herein, which first fusion protein comprises a chimeric GPCR of the invention. The invention also relates to liposome or vesicle a cell or cell line that comprises a second fusion protein as described herein. In yet another aspect, the invention relates to a liposome or vesicle that comprises both a first fusion protein as described herein and a second fusion protein as described herein. In aspects and embodiments where the second ligand does not form part of the second fusion protein, such a liposome or vesicle may also contain a suitable second ligand (which again is preferably a naturally occurring G-protein or a synthetic or semi-synthetic analog or derivative of a G-protein, in particular when the ICLs that are present in a chimeric GPCR of the invention form a functional G-protein binding site).

Thus, as further described herein and as will be illustrated by means of the appended non-limiting Figures, and depending on whether the second ligand is part of the second fusion protein or not, the invention envisages at least three preferred embodiments of the methods and arrangements of the invention in which a chimeric GPCR of the invention is used.

In a first such preferred embodiment (schematically shown in FIG. 1), the second binding member of the binding pair will be suitably fused or linked (either directly or via a suitable linker or spacer) to the second ligand. According to this preferred embodiment, an arrangement for performing the methods of the invention may thus comprise at least the following elements:
a boundary layer that separates a first environment from a second environment;
a binding pair that consists of at least a first binding member and a second binding member, which binding pair is capable of generating a detectable signal;
a translayer protein that is suitably fused or linked (either directly or via a suitable linker or spacer) to one of the binding members of said binding pair;
a first ligand for the translayer protein that is present in the first environment; and
a second ligand for the translayer protein that is present in the second environment and that is suitably fused or linked (either directly or via a suitable linker or spacer) to the other binding member of said binding pair;
   in which said translayer protein is a chimeric GPCR of the invention as described herein and in which the elements of the arrangement are arranged with respect to each other (and where applicable operably linked to and/or associated with each other) in the manner as further described herein. As further described herein, in this embodiment, the second ligand is preferably a binding domain or binding unit that can bind to a binding site on said chimeric GPCR that comprises at least one of said intracellular loops (as described herein) and is more preferably a conformation-inducing binding domain or binding unit (also as described herein) and may in particular be an ISVD and more in particular a conformation-inducing ISVD.

In particular, as further described herein, such an arrangement may comprise the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment; and
a second fusion protein comprising a second ligand for the translayer protein and the other binding member of said binding pair, which second fusion protein is present in the second environment;
   in which said translayer protein is a chimeric GPCR of the invention as described herein and in which the elements of the arrangement are arranged with respect to each other (and where applicable operably linked to and/or associated with each other) in the manner as further described herein. As further described herein, in this embodiment, the second ligand that is present in the second fusion protein is preferably a binding domain or binding unit that can bind to a binding site on said chimeric GPCR that comprises at least one of said intracellular loops (as described herein) and is more preferably a conformation-inducing binding domain or binding unit (also as described herein) and may in particular be an ISVD and more in particular a conformation-inducing ISVD.

In a second such preferred embodiment (schematically shown in FIG. 2), the second binding member of the binding pair will be suitably fused or linked (either directly or via a suitable linker or spacer) to a binding domain or binding unit that does not bind directly to the translayer protein (i.e. to the chimeric GPCR), but instead binds to the second ligand (which in turn can bind to the chimeric GPCR). According to this preferred embodiment, an arrangement for performing the methods of the invention may thus comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment;
a second ligand for the translayer protein that is present in the second environment; and
a second fusion protein that is present in the second environment and that comprises a binding domain or binding unit that can bind to the second ligand, which binding domain or binding unit is suitably fused or linked (either directly or via a suitable linker or spacer) to the other binding member of said binding pair;
   in which said translayer protein is a chimeric GPCR of the invention as described herein and in which the elements of the arrangement are arranged with respect to each other (and where applicable operably linked to and/or associated with each other) in the manner as further described herein.

It will be clear to the skilled person that in this second embodiment, the binding domain or binding unit that is present in the second fusion protein will bind “indirectly” to the chimeric GPCR, i.e. by binding to the second ligand which binds to the chimeric GPCR. Again, said binding domain or binding unit is preferably an immunoglobulin single variable domain as further described herein. Also, in this embodiment, the second ligand can be any suitable ligand for the chimeric GPCR as further described herein, but as mentioned is preferably a naturally occurring G-protein or a synthetic or semi-synthetic analog or derivative of a G-protein, in particular when the ICLs that are present in a chimeric GPCR of the invention form a functional G-protein binding site.

In a third preferred embodiment (schematically shown in FIG. 3), the second binding member of the binding pair will be suitably fused or linked (either directly or via a suitable linker or spacer) to a binding domain or binding unit that does not bind directly to the translayer protein (i.e. to the chimeric GPCR), but instead binds to a protein complex that comprises at least the second ligand for the chimeric GPCR (which protein complex may either bind to, or be bound by, the chimeric GPCR and/or comprise the chimeric GPCR). According to this preferred embodiment, an arrangement for performing the methods of the invention may thus comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment;
a protein complex that at least comprises a second ligand for the translayer protein, which protein complex is present in the second environment; and
a second fusion protein that is present in the second environment and that comprises a binding domain or binding unit that can bind to the protein complex, which binding domain or binding unit is suitably fused or linked (either directly or via a suitable linker or spacer) to the other binding member of said binding pair;
   in which said translayer protein is a chimeric GPCR of the invention as described herein and in which the elements of the arrangement are arranged with respect to each other (and where applicable operably linked to and/or associated with each other) in the manner as further described herein.

It will be clear to the skilled person that in this third embodiment, the binding domain or binding unit that is present in the second fusion protein will bind “indirectly” to the chimeric GPCR, i.e. by binding to a protein complex that comprises the second ligand. Again, said binding domain or binding unit is preferably an immunoglobulin single variable domain as further described herein, and the second ligand can be any suitable ligand for the that can be part of a protein complex as further described herein, but as mentioned is preferably a G-protein complex, in particular when the ICLs that are present in a chimeric GPCR of the invention form a functional G-protein binding site.

More generally, the arrangements as described herein that comprise a chimeric GPCR of the invention will usually, and preferably, at least comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment;
a second ligand for the translayer protein that is present in the second environment; and
a second fusion protein that comprises the other binding member of said binding pair (i.e. such that said other member of the binding pair is also present in the second environment);
   in which said translayer protein is a chimeric GPCR of the invention as described herein and in which the elements of the arrangement are arranged with respect to each other (and where applicable operably linked to and/or associated with each other) in the manner as further described herein. In particular:
in the first preferred embodiment described herein, the second fusion protein will comprise the other binding member of said binding pair and the second ligand;
in the second preferred embodiment described herein, the second fusion protein will comprise the other binding member of said binding pair and a binding domain or binding unit that can bind to the second ligand; and
in the third preferred embodiment described herein, the second fusion protein will comprise the other binding member of said binding pair and a binding domain or binding unit that can bind to a protein complex that comprises at least the second ligand.

The invention will now be illustrated by means of the further description herein, the Experimental Part below, and the appended non-limiting Figures. In the Figures:
a) FIG. 1 schematically shows a first arrangement of the invention, in which the second ligand (indicated as (4) in FIG. 1), forms part of the second fusion protein (which in the embodiment shown in FIG. 1 is formed by the second ligand (4), the linker (11) and the second member (7) of the binding pair (6/7)) binds directly (as defined herein) to the translayer protein (2). In the set-up shown in FIG. 1:
   the boundary layer is indicated as (1);
   the first environment is indicated as [A];
   the second environment is indicated as [B];
   the translayer protein (i.e. the chimeric GPCR of the invention) is indicated as (2);
   the first ligand is indicated as (3);
   the first binding site on the translayer protein (2) that is exposed to the first environment [A] and to which the first ligand (3) can bind is indicated as (8). As described herein, said first binding site (8) on the translayer protein is the extracellular binding site (as defined herein) on the chimeric GPCR of the invention;
   the second ligand is indicated as (4). As described herein, said second ligand is preferably a binding domain or binding unit that can bind to a binding site on said chimeric GPCR that comprises at least one of said intracellular loops (as described herein) and is more preferably a conformation-inducing binding domain or binding unit (also as described herein) and may in particular be an ISVD and more in particular a conformation-inducing ISVD;
   the second binding site on the translayer protein (2) that is exposed to the second environment [B] and to which the second ligand (4) can bind is indicated as (9). As described herein, said second binding site (9) on the translayer protein is the intracellular binding site (as defined herein) on the chimeric GPCR of the invention and comprises at least one of the ICLs from the second GPCR;
   the binding pair that can generate a detectable signal is indicated as (6/7) and consists of a first binding member (6) linked to the translayer protein (2) (either directly of via linker or spacer (10)) and a second binding member (7) linked to the second ligand (4) (either directly of via linker or spacer (11));
   the first fusion protein comprises the translayer protein (2) that is fused, either directly or via the linker (10), to the first binding member (6);
   the second fusion protein comprises the second ligand (4) that is fused, either directly or via the linker (11), to the second binding member (7); and
   the first and second fusion proteins are arranged with respect to each other and with respect to the boundary layer (1) in such a way that, when the second ligand (4) binds to the translayer protein (2) (i.e. directly via the binding site (9)), the first binding member (6) and the second binding member (7) can come into contact or close proximity with/to each other (or otherwise suitably associate) so as to generate a detectable signal (indicated with the flash symbol in FIG. 1).
b) FIG. 2 schematically shows a second arrangement of the invention, in which the second ligand (indicated as (4) in FIG. 2) is separate from the second fusion protein (which in the embodiment shown in FIG. 2 is formed by the binding domain (5), the linker (11) and the second member (7) of the binding pair (6/7)) and in which the binding domain (5) which is present in the second fusion protein binds indirectly (as defined herein, and in the case of FIG. 2 via the second ligand (4)) to the translayer protein (2). In the set-up shown in FIG. 2:

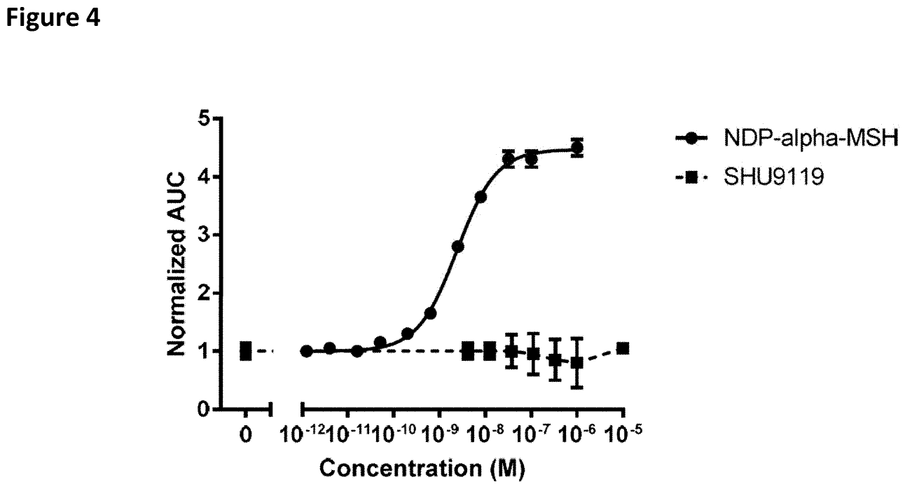

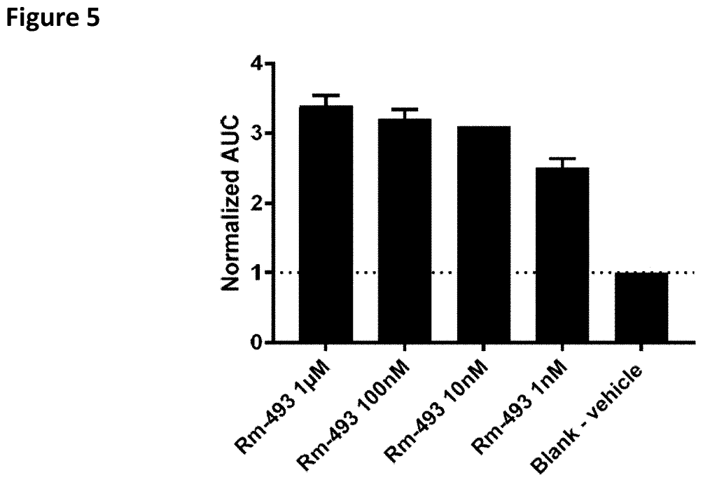

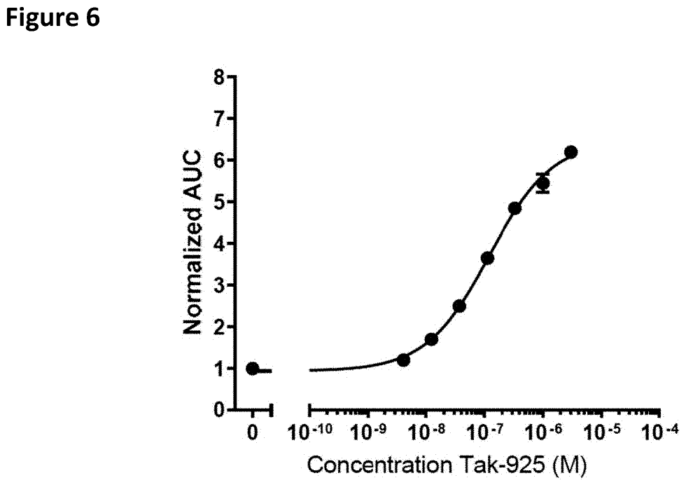

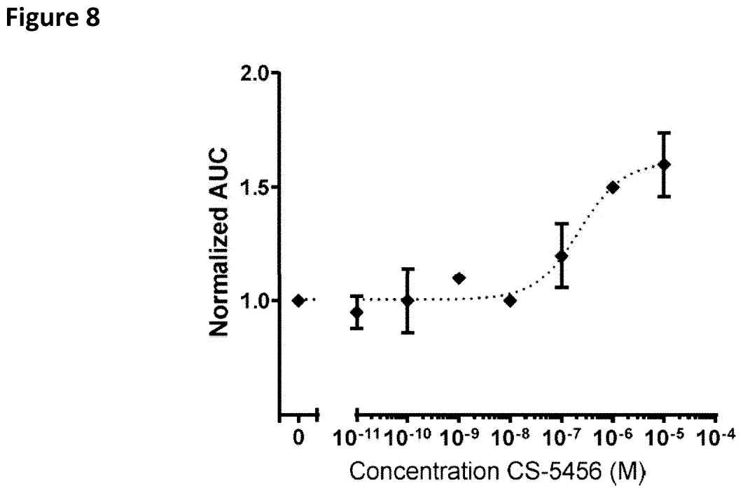

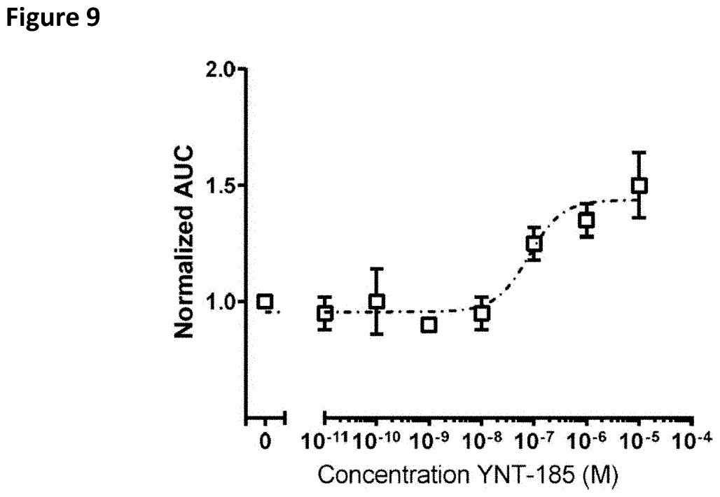

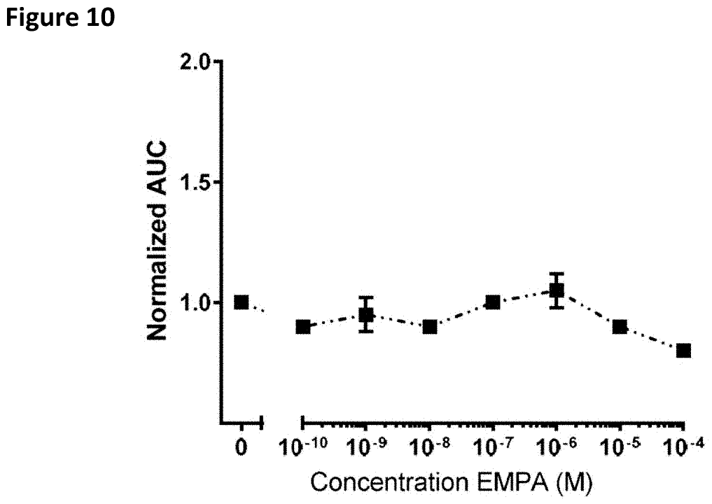

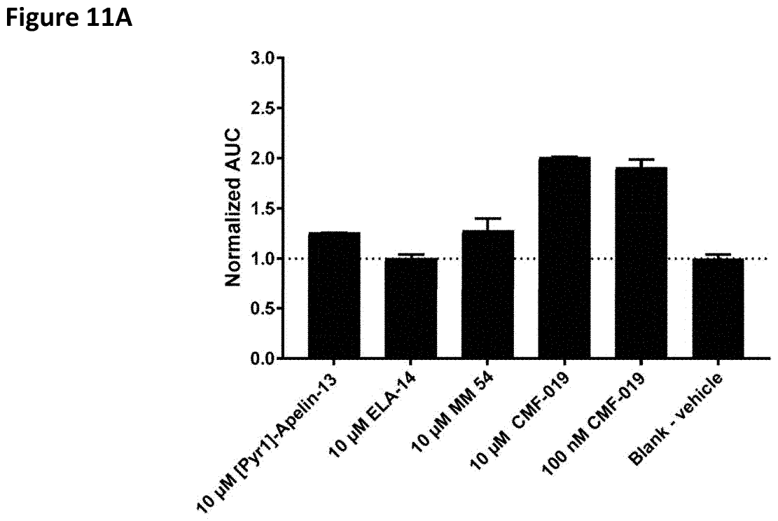

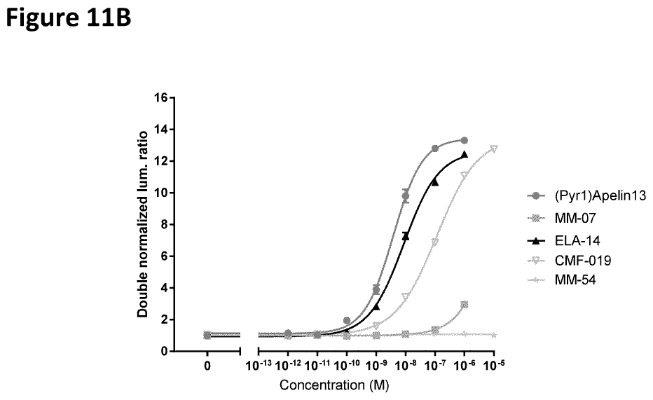

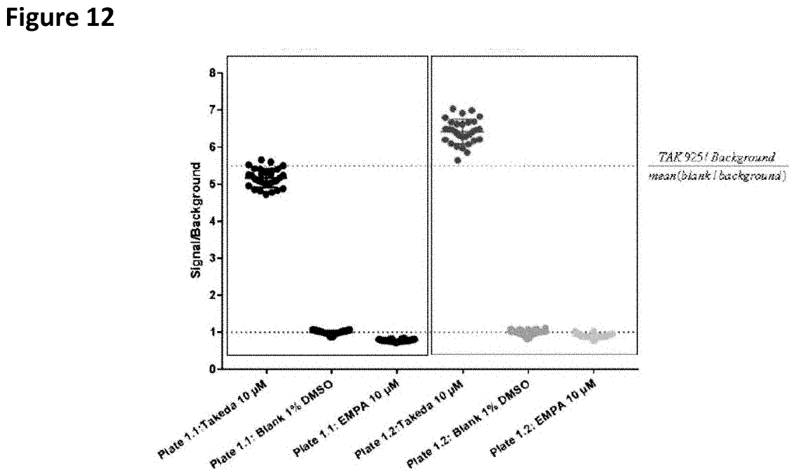

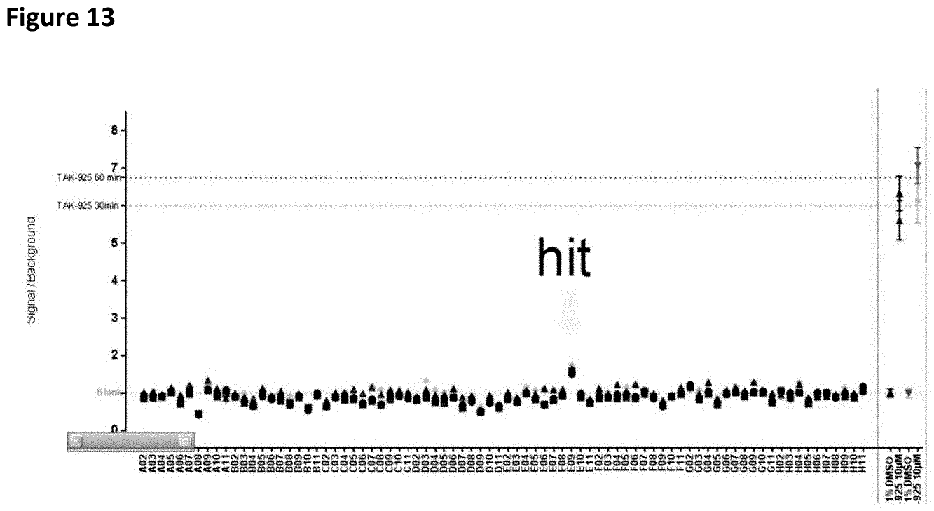

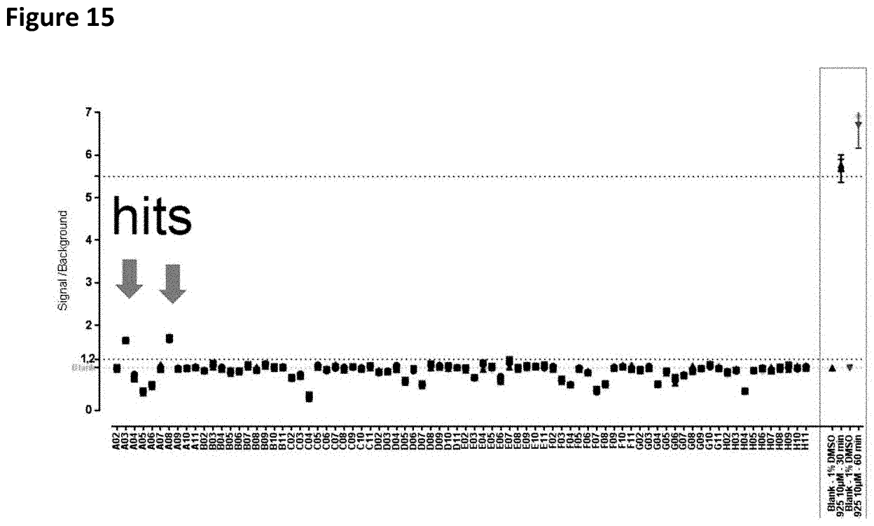

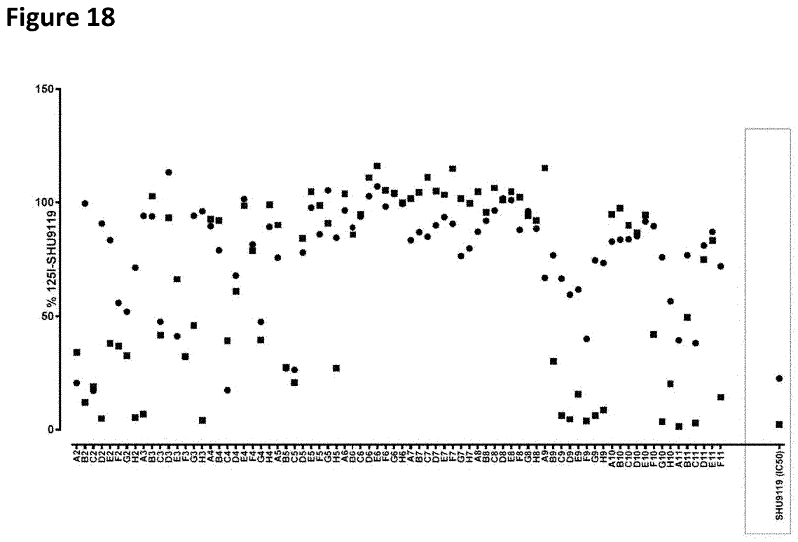

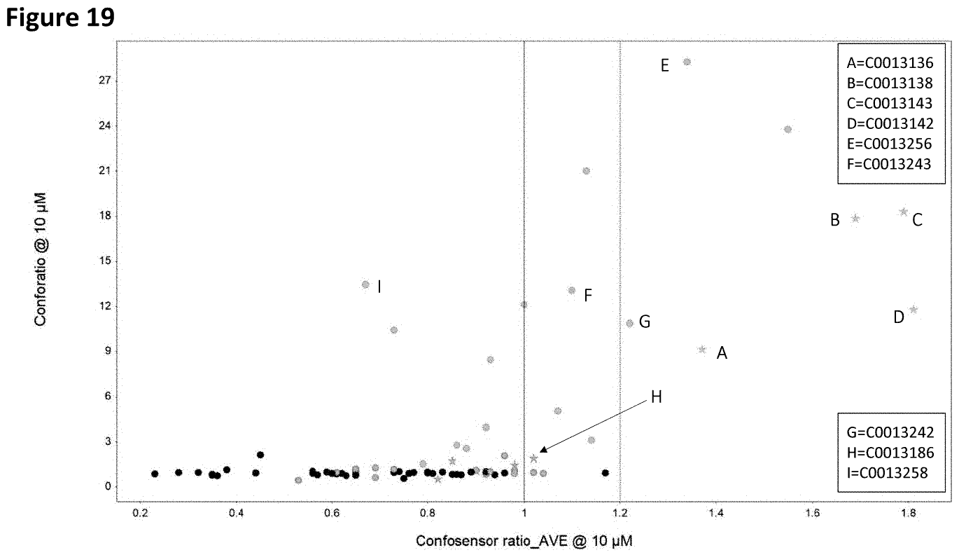

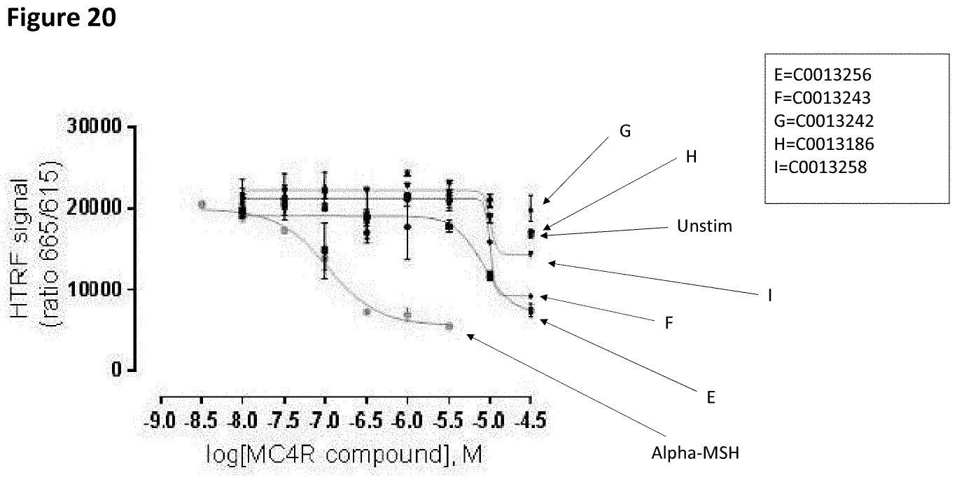

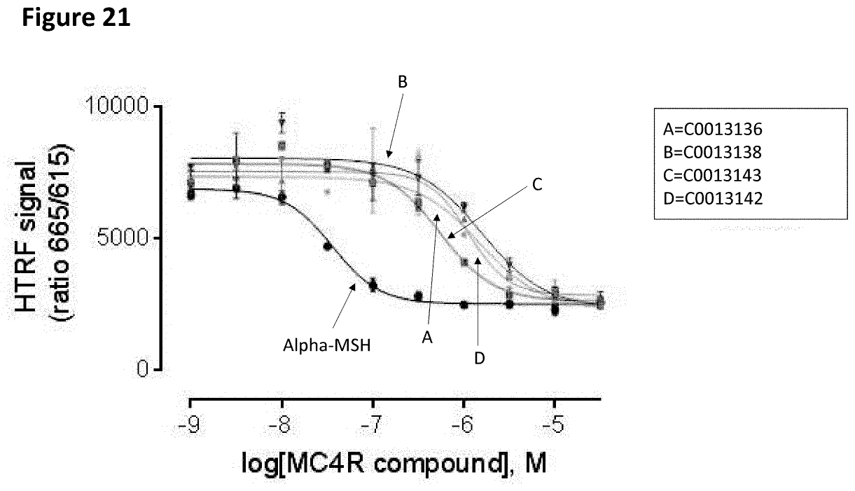

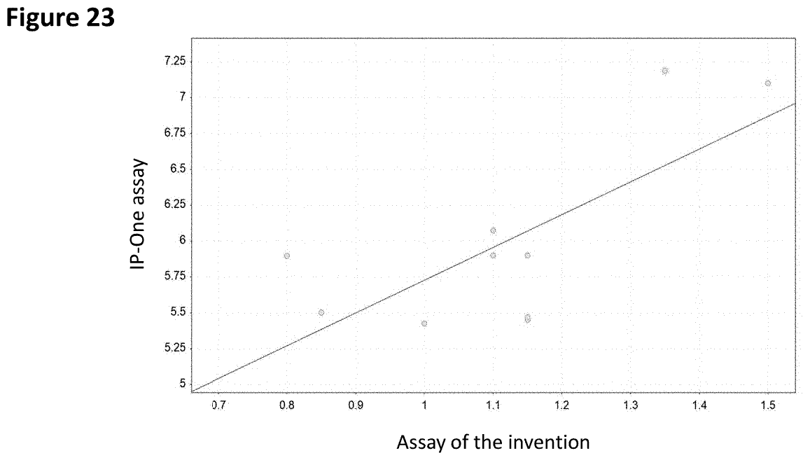

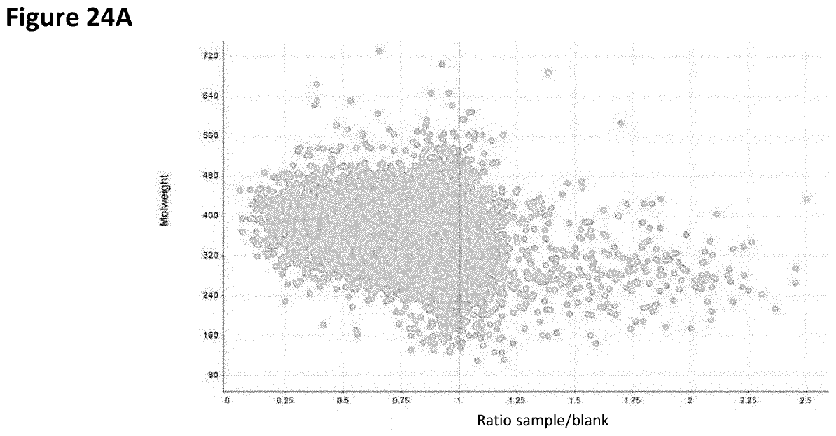

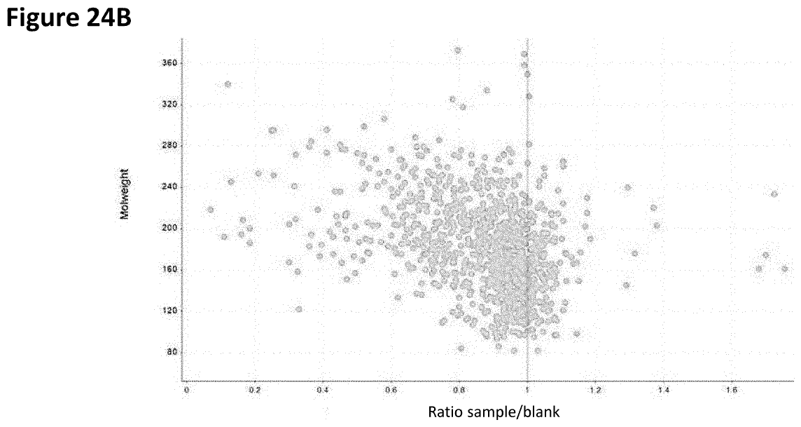

the boundary layer is indicated as (1);
the first environment is indicated as [A];
the second environment is indicated as [B];
the translayer protein (i.e. the chimeric GPCR of the invention) is indicated as (2);
the first ligand is indicated as (3);
the first binding site on the translayer protein (2) that is exposed to the first environment [A] and to which the first ligand (3) can bind is indicated as (8). As described herein, said first binding site (8) on the translayer protein is the extracellular binding site (as defined herein) on the chimeric GPCR of the invention;
the second ligand is indicated as (4);
the second binding site on the translayer protein (2) that is exposed to the second environment [B] and to which the second ligand (4) can bind is indicated as (9). As described herein, said second binding site (9) on the translayer protein is the intracellular binding site (as defined herein) on the chimeric GPCR of the invention and comprises at least one of the ICLs from the second GPCR;
the binding domain or binding unit that can bind to the second ligand (4) is indicated as (5);
the binding pair that can generate a detectable signal is indicated as (6/7) and consists of a first binding member (6) linked to the translayer protein (2) (either directly of via linker or spacer (10)) and a second binding member (7) linked to the binding domain or binding unit (5) (either directly of via linker or spacer (11));
the first fusion protein comprises the translayer protein (2) that is fused, either directly or via the linker (10), to the first binding member (6);
the second fusion protein comprises the binding domain (5) that is fused, either directly or via the linker (11), to the second binding member (7); and
the first and second fusion proteins are arranged with respect to each other and with respect to the boundary layer (1) in such a way that, when the binding domain (5) binds to the translayer protein (2) (i.e. indirectly by binding to the second ligand (4) which in turn binds to the translayer protein (2) via the binding site (9)), the first binding member (6) and the second binding member (7) can come into contact or close proximity with/to each other (or otherwise suitably associate) so as to generate a detectable signal (indicated with the flash symbol in FIG. 2).
c) FIG. 3 schematically shows a third arrangement of the invention, in which the second ligand (indicated as (4) in FIG. 3) is separate from the second fusion protein and forms part of a protein complex (12) that is formed by the second ligand (4) and one or more further proteins (in the case of FIG. 3, for illustration purposes, said complex is exemplified as comprising the second ligand (4) and two further proteins (4a) and (4b)—see also the insert in FIG. 3). In the embodiment shown in FIG. 3, the second ligand (4) is again separate from the second fusion protein (which in the embodiment shown in FIG. 3 is formed by the binding domain (5), the linker (11) and the second member (7) of the binding pair (6/7)) and the binding domain (5) that is present in the second fusion protein binds indirectly (as defined herein, and in the case of FIG. 3 via the protein complex (12)) to the translayer protein (2). In the set-up shown in FIG. 3:
   the boundary layer is indicated as (1);
   the first environment is indicated as [A];
   the second environment is indicated as [B];
   the translayer protein (i.e. the chimeric GPCR of the invention) is indicated as (2);
   the first ligand is indicated as (3);
   the first binding site on the translayer protein (2) that is exposed to the first environment [A] and to which the first ligand (3) can bind is indicated as (8). As described herein, said first binding site (8) on the translayer protein is the extracellular binding site (as defined herein) on the chimeric GPCR of the invention;
   the second ligand is indicated as (4), and forms a complex (12) with one or more other proteins (for illustration purposes, in FIG. 3, the complex (12) is represented as a complex comprising three proteins/subunits, namely the second ligand (4) and two further subunits (4a) and (4b)—see also the insert in FIG. 3);
   the second binding site on the translayer protein (2) that is exposed to the second environment [B] and to which the complex (12) can bind is indicated as (9). As described herein, said second binding site (9) on the translayer protein is the intracellular binding site (as defined herein) on the chimeric GPCR of the invention and comprises at least one of the ICLs from the second GPCR;
   the binding domain or binding unit that can bind to complex (12) is indicated as (5);
   the binding pair that can generate a detectable signal is indicated as (6/7) and consists of a first binding member (6) linked to the translayer protein (2) (either directly of via linker or spacer (10)) and a second binding member (7) linked to the binding domain or binding unit (5) (either directly of via linker or spacer (11));
   the first fusion protein comprises the translayer protein (2) that is fused, either directly or via the linker (10), to the first binding member (6);
   the second fusion protein comprises the binding domain (5) that is fused, either directly or via the linker (11), to the second binding member (7); and
   the first and second fusion proteins are arranged with respect to each other and with respect to the boundary layer (1) in such a way that, when the binding domain (5) binds to the translayer protein (2) (i.e. indirectly by binding to the complex (12) which in turn binds to the translayer protein (2) via the binding site (9)), the first binding member (6) and the second binding member (7) can come into contact or close proximity with/to each other (or otherwise suitably associate) so as to generate a detectable signal (indicated with the flash symbol in FIG. 3).
d) FIG. 4 is a graph showing the dose response curves for the indicated compounds obtained using the recombinant MC4R screening assay described in Example 2;
e) FIG. 5 is a graph showing assay results obtained using the recombinant MC4R screening assay described in Example 2;
f) FIGS. 6 to 10 are graphs showing the dose response curves for the indicated compounds obtained using the recombinant OX2R screening assay described in Example 3;
g) FIGS. 11A and B are graphs showing assay results obtained using the two recombinant APJ receptor screening assays described in Example 4. FIG. 11A shows the results obtained with a recombinant apelin receptor having the ICLs of a mu-opioid receptor (MOR) and FIG. 11B shows the results obtained with a recombinant apelin receptor having the ICLs from a beta-2AR receptor;
h) FIGS. 12 to 15 show the results from the compound library screening performed in Example 5;
i) FIG. 16A to C show alignments of two MC4R chimers referred to in the Experimental part (SEQ ID NOs: 11 and 12) and the amino acid sequences of human MC4R (SEQ ID NO: 13) and beta-2-adrenergic receptor (SEQ ID NO: 14) from which said chimers were derived;
j) FIG. 17 is a schematic representation of the tertiary structure of the amino acid sequence of MC4R-B2AR Chimer 1 (SEQ ID NO: 11), showing the N- and C-terminal sequence, the ECLs, ICLs and TMs. The amino acid residues in the chimeric GPCR that are derived from B2AR (the ICLs and some ICL-flanking residues) have been shaded in gray;
k) FIG. 18 is a graph showing a schematic representation of the testing of a series of compounds (A2 to F11, indicated on the x-axis) in a radioligand assay using wild-type MC4R (SEQ ID NO: 13) and MC4R-B2AR Chimer 1 (SEQ ID NO: 11). For each compound, displacement was measured (see Example 6) with respect to wildtype (value indicated with a dot) and with respect to the chimer (value indicated with a square);
l) FIG. 19 is a graph showing the results obtained for a series of compounds (with each data point in the graph representing a different compound) in a radioligand assay and an assay set-up as shown in FIG. 1 using an MC4R/B2AR chimer. Results from the radioligand assay are set out along the y-axis (“Conforatio@10micromolar”) and results from the ConfoSensor assay are set out along the x-axis (“ConfoSensor ratio”). Compounds selected for further testing in a cAMP cellular assay using wild-type MC4R are indicated as A to I.
m) FIGS. 20 and 21 are graphs from two separate experiments showing a read-out in a cAMP cellular assay using wild-type MC4R for Alpha-MSH (reference), unstimulated cells (“Unstim”), and compounds A to I that were selected for testing in the cAMP assay based on the data shown in FIG. 19;
n) FIG. 22 shows the amino acid sequence of human β2AR (UniProt P07550, see SEQ ID NO: 17 and FIG. 22);
o) FIG. 23 is a plot obtained in Example 9 comparing results from an OX2 assay of the invention (using a recombinant OX2 fusion) and an OX2 IP-One assay, with the x-axis representing the data obtained in the assay of the invention, the y-axis representing the data obtained in the IP-One assay and each dot representing the results for a single compound.
p) FIGS. 24A and 24B are plots obtained in Example 10 when a large compound library was screened against a recombinant OX2 receptor using an assay of the invention. FIG. 24A shows the results obtained when the compounds were tested at 30 μM) and FIG. 24B shows the results obtained when the compounds tested at 200 μM), with the x-axis representing the ratio of the signal obtained with the compound tested (“sample”) vs signal given by the carrier solvent (“blank”) and each dot representing the result obtained for a single compound.

From the Figures and the further description herein, it will be clear to the skilled person that some elements of an arrangement that comprises a chimeric GPCR of the invention (such as the boundary layer, the chimeric GPCR, the binding pair, any linkers and the first ligand) will be present in the various aspects and embodiments of the invention as contemplated herein. Thus, when a detailed description is given herein of any such element (including any preferences for any such element), it should be understood that such description applies to all aspects and embodiments of the invention in which such element is present or used, unless explicitly stated otherwise herein.

In the methods and arrangements of the invention, the boundary layer (1) can be any layer (such as a wall or a membrane) that is suitable to separate the first environment [A] from the second environment [B] (either in a suitable in vitro system or a suitable in vivo system).

For example, in one preferred aspect of the invention in which the methods of the invention are performed in a suitable cell or cell line (as further described herein), the boundary layer (1) is the cell membrane or cell wall of the cell or cell line that is used in the methods of the invention. In this aspect, the environment [A] is preferably the extracellular environment and the environment [B] is preferably the intracellular environment. Also, in this aspect, the first ligand (3) is preferably present in the extracellular environment and the second ligand (4) is preferably present in the intracellular environment. Also, the first and second binding members (6) and (7) and the second fusion protein are preferably also present in the intracellular environment,

In another preferred aspect of the invention in which the methods of the invention are performed in a suitable vesicle or liposome (as further described herein), the boundary layer (1) is the membrane or wall of the vesicle or liposome. In this aspect, the environment [A] is preferably the environment outside of the vesicle or liposome and the environment [B] is preferably the environment inside the vesicle or liposome. Also, in this aspect, the first ligand (3) is preferably present in the environment outside the vesicle or liposome and the second ligand (4) is preferably present in the environment inside the vesicle or liposome. Also, the first and second binding members (6) and (7) and the second fusion protein are preferably also present in the environment inside the vesicle or liposome.

However, it should be understood that, although the invention in some preferred aspects is performed using cells, liposomes or other suitable vesicles, the invention in its broadest sense is not limited to the use of cells or vesicles but can be performed in any other suitable arrangement in which a boundary layer (1) is used to suitably separate a first environment [A] from a second environment [B]. For example, the boundary layer may also be a part or fragment of a cell wall or cell membrane that is present in a membrane extract, for example a membrane extract that is obtained from whole cells by a technique known per se such as suitable osmotic and/or mechanic techniques known per se.

Thus, the boundary layer (1) can be any suitable layer, wall or membrane, and in particular a biological wall or membrane (such as a cell wall or cell membrane, or a part or fragment thereof) or the wall or membrane of a liposome or other suitable vesicle. In particular, the boundary layer (1) can be a suitable lipid bilayer such as a phospholipid bilayer. When the boundary layer (1) is the wall or membrane of a vesicle or liposome, it can be unilamellar or multilamellar. Also, as further described herein, when the boundary layer (1) is a cell membrane or cell wall, it is preferably the wall or membrane of a cell or cell line that suitably expresses (as defined herein) the translayer protein (2) (i.e. the chimeric GPCR of the invention) and in particular suitably expresses a (first) fusion protein as described herein that comprises the translayer protein (2).

As schematically illustrated by the non-limiting FIGS. 1, 2 and 3, the boundary layer (1) contains the translayer protein (2) (i.e. the chimeric GPCR of the invention), which spans the boundary layer (1) such that:
the first binding site (8) for the first ligand (3) (i.e. the extracellular binding site on the chimeric GPCR of the invention, as described herein) extends out (as defined herein) into the first environment [A] (i.e. such that the first binding site (8) is accessible for binding by the first ligand (3) when said first ligand is present in the first environment [A]);
   and also such that
the second binding site (9) for the second ligand (4) (i.e. the intracellular binding site on the chimeric GPCR of the invention, as described herein) extends out (as defined herein) into the second environment [B] (i.e. such that the second binding site (9) is accessible for binding by the second ligand (4) when said second ligand is present in the second environment [B]).

In the methods and arrangements of the invention, the chimeric GPCR of the invention (which serves as the translayer protein (2) in said methods and arrangements) is such (and/or is provided and/or arranged in such a way with respect to the boundary layer) that it spans the boundary layer (1), such that at least one part of the amino acid sequence of the chimeric GPCR (and in particular at least one ECL and preferably all ECLs) extends out (as defined herein) from the boundary layer (1) into the first environment [A] and such that at least one other part of the amino acid sequence of the chimeric GPCR (and in particular at least one ICL and preferably all ICLs) extends out (as defined herein) from the boundary layer (1) into the second environment [B]. In this context, when a part of the amino acid sequence of the chimeric GPCR of the invention (such as an ECL or ICL, respectively) is said to “extend out” from the boundary layer (1) into an environment (i.e. into the first environment [A] or the second environment [B]), this should generally be understood to mean that said part of the sequence is exposed to said environment and/or is accessible for binding by a ligand, compound or other chemical entity that is present in said environment. Accordingly, in the methods and arrangements that employ a chimeric GPCR of the invention, at least one part of the amino acid sequence of the chimeric GPCR (such as an epitope or binding site) should be accessible for binding by a ligand, compound or other chemical entity that is present in the first environment (and in particular, for binding by the first ligand (3)) and at least one other part of the amino acid sequence of the translayer protein (such as another epitope or binding site) should be accessible for binding by a ligand, compound or other chemical entity that is present in the second environment (and in particular, for binding by the second ligand (4))). In this respect, it should also be noted that the wording “accessible for binding” should generally be taken to mean that a ligand, compound or other chemical entity that is present in the relevant environment can bind to a binding pocket or binding site on or within the chimeric GPCR of the invention (such as the extracellular binding site, as defined herein), even if the actual binding site or binding pocket lies deep(er) within the structure of the translayer protein (even such that the actual binding site or binding pocket is located within a part of the translayer protein that itself does not physically extend out beyond the boundary layer). Reference is for example made to the publication by Chevillard (cited herein) which shows that the binding sites on GPCRs for fragments that are used in FBDD screening techniques may lie deep within the GPCR structure (see for example FIG. 2 on page 1120) and not be on the surface of the GPCR, but nevertheless are accessible for fragment binding. Reference is also made to the teachings on GPCR structure, GPCR signaling mechanisms and GPCR ligand binding sites from some of the other scientific references cited herein,

Also, in the present description and claims, when any binding domain, binding unit, epitope, binding site, ligand, protein or other compound or chemical or other structural entity (such as a protein complex) is said to be “present in” an environment (i.e. in the first environment [A] or the second environment [B]), this should generally be understood to mean that said binding domain, binding unit, epitope, binding site, ligand, protein or other compound or chemical or structural entity is exposed to said environment and/or is accessible for binding by another domain, ligand, protein or compound that is present in said environment. Thus, for example, a compound or ligand that is present in an environment may either be “free-floating” in said environment (i.e. not be bound or anchored to any other protein or structure) or may be anchored to the boundary layer or fused to another protein (which other protein may be anchored to the boundary layer). Similarly, a binding domain or binding unit that is present in an environment may be part of a larger protein or structure (such as a fusion protein), which larger structure may be free-floating in said environment or be anchored to the boundary layer or to another structure, as long as the binding domain or binding unit is accessible for binding by another domain, ligand, protein or compound that is present in said environment. Also, an epitope or binding site that is present in an environment may be part of a larger protein or structure, which larger protein or structure may again be free-floating in said environment or be anchored to the boundary layer or to another structure, as long as the epitope or binding site is accessible for binding by another domain, ligand, protein or compound that is present in said environment.

The part or parts of the chimeric GPCR of the invention that extend out into the first environment [A] can be any loop, epitope (linear or conformational), binding site or other part(s) of the amino acid sequence of the translayer protein, and similarly the part or parts of the translayer protein that extend out into the second environment [B] can also be any loop, epitope (linear or conformational), binding site or other part(s) of the amino acid sequence of the translayer protein (but will be different from the part(s) that extend out into the first environment), but as described herein preferably at least one of the ECLs (and more preferably all of the ECLs, and in particular the extracellular binding site) of the chimeric GPCR of the invention will extend out into the first environment [A] and preferably at least one of the ICLs (and more preferably all of the ICLs, and in particular the intextracellular binding site) of the chimeric GPCR of the invention will extend out into the first environment [B].

Generally, the translayer protein (2) (i.e. the chimeric GPCR of the invention) will usually be attached to and/or anchored in the boundary layer (1), for example in a manner that is known per se for GPCRs. As further described herein, this can for example be achieved by suitably expressing (as defined herein) a nucleotide sequence or nucleic acid that expresses the first fusion protein in a suitable host cell such that the chimeric GPCR of the invention becomes suitably anchored in the wall or membrane of said cell. When the method of the invention is performed using a liposome or vesicle, this can be achieved by suitably forming said liposome or vesicle in the presence of the first fusion protein such that the chimeric GPCR becomes suitably anchored into the wall or membrane of the liposome or vesicle.

Also, when the methods of the invention are performed in cells, the arrangement of the N-terminus and the C-terminus of the chimeric GPCR of the invention relative to the wall or membrane of the cell used are preferably the same as the arrangement of said termini in of the first and second GPCRs from which the ECLs and ICLs, respectively, have been derived (i.e. when said first and second GPCRs are in their native cellular environment). This also applies when the C-terminal end of the chimeric GPCR has been derived from the second GPCR instead of the first GPCR.

When the methods of the invention are to be performed in liposomes or vesicles, it may be that the liposomes or vesicles may be a mixture of liposomes/vesicles in which the chimeric GPCR of the invention is arranged in a way that is essentially the same as the way that the first and second GPCRs from which the ECLs and ICLs, respectively, have been derived are arranged with respect to the cell wall or cell membrane in their native environment (i.e. with the N-terminus and the extracellular loop(s) extending to the outside of the vesicle and the C-terminus and the intracellular loop(s) extending to the inside of the vesicle) and vesicles/liposomes in which the protein is arranged the other way around. Usually, this will not affect the performance of the system or set-up described herein.

As is known for naturally occurring GPCRs, the chimeric GPCR of the invention should most preferably be such that it exists (i.e. can take on) two or more conformations (such as a basal state/conformation, an active state/conformation and/or an inactive state/conformation, and/or a ligand-bound or ligand-free conformation) and/or such that it that can undergo a conformational change (and in particular, a functional conformational change). In particular, the chimeric GPCR of the invention should be such that it can take on at least one functional conformation and at least one non-functional conformation (such as a basal conformation) and/or that can undergo a conformational change from a non-functional conformation into a functional conformation; and more in particular such that it that can take on an active (or more active) conformation and an inactive (or less active) conformation and/or that can undergo a conformational change from an inactive (or less active) conformation into an active (or more active) conformation. Also, the chimeric GPCR is preferably such that it can take on at least one ligand-bound (and in particular agonist-bound) conformation and at least one ligand-free conformation. More in particular, the chimeric GPCR may be such that it can take on at least one ligand-bound (and in particular agonist-bound) conformation that is an active or functional conformation.

As described herein, a particular class of functional conformations of (transmembrane) proteins (such as certain GPCRs) is referred to/defined as “druggable conformation”. Thus, in one specific aspect, the chimeric GPCR is such that it can take on at least one such druggable conformation (which will often be an active conformation, although the invention is not limited to use with druggable conformations that are active conformations) and at least one conformation that is not a druggable conformation (which will often be an inactive conformation) and/or a such that it can undergo a conformational change from a non-druggable conformation to a druggable conformation.

In particular, the chimeric GPCR is preferably such that it undergoes a conformational change upon binding of a ligand (and in particular an agonist) to the protein. This conformational change upon binding of the ligand can for example be a conformational change from an active conformation into an inactive conformation or from a functional conformation to a non-functional conformation, but is preferably a change from a non-functional conformation to functional conformation and/or an inactive conformation to an active conformation. In a particular aspect, it is a change from a non-druggable conformation into a druggable conformation.

For example, the conformational change of the chimeric GPCR may be a change from a conformation that is essentially not capable of binding G-protein into a conformation that binds G-protein (or is capable of being bound by G-protein), and may in particular be a change from a conformation that is essentially not capable (or less capable) of binding the conformation-inducing binding domain or binding unit into a conformation that binds the conformation-inducing binding domain or binding unit (or is more capable of being bound by the conformation-inducing binding domain or binding unit).

As mentioned herein, a ligand that is capable of eliciting a conformational change in a GPCR from a non-functional state into a functional state (for example from an inactive state such as a basal state into an active state) is also referred to herein as an “agonist” of said GPCR. In particular, an “agonist” of a GPCR may be capable of eliciting a conformational change from a conformation that is essentially not capable of binding G-protein into a conformation that binds G-protein.

In one preferred aspect, the chimeric GPCR such that it undergoes (or is capable of undergoing) a conformational change (as described herein) when the first ligand (3) binds to it and conversely the first ligand (3) is such that it can invoke a conformational change in chimeric GPCR when it binds to it (and/or the invention is used to identify such first ligands). Again, in one more preferred aspect, said conformational change is a change from an inactive or less active state to a functional or (more) active state and the first ligand (3) used is such that, when it binds to the chimeric GPCR, it can invoke a conformational change in the chimeric GPCR from an inactive or less active state into a functional or (more) active state. Again, said conformational change upon binding of the first ligand (3) may be a change from a conformation that is essentially not capable of binding G-protein into a conformation that binds G-protein.

As also further described herein, the chimeric GPCR may be such that it can form a complex with a first and a second ligand. In this respect, it is known that most naturally occurring GPCRs form a complex with an extracellular ligand and the G-protein (which is the most common native intracellular ligand for a GPCR), and that such a complex is stabilized by the G-protein binding to the intracellular conformational epitope of the GPCR. Similarly, in the invention, the second ligand is preferably such that it stabilizes (the formation of) a complex of the chimeric GPCR, the first ligand and the second ligand. As described herein. for this purpose, the second ligand may be the G-protein that is associated with the second GPCR (i.e. the GPCR from which the ICLs of the chimeric GPCR have been derived) in its native environment (i.e. with signal transduction by the GPCR), another naturally occurring G-protein that is capable of binding to the chimeric GPCR and stabilizing the formation of the aforementioned complex, or a synthetic or semi-synthetic analog or derivative of a GPCR that is capable binding to the chimeric GPCR and stabilizing the formation of the aforementioned complex. As also described herein, the second ligand is preferably a binding domain or binding unit that can bind to a binding site on said chimeric GPCR that comprises at least one of said intracellular loops (as described herein) and is more preferably a conformation-inducing binding domain or binding unit (also as described herein) and may in particular be an ISVD and more in particular a conformation-inducing ISVD.

As further described herein, and as schematically shown in FIGS. 1 to 3, in the arrangements of the invention, the translayer protein (2) (i.e. the chimeric GPCR of the invention) is usually, and preferably, fused or linked, either directly or via a suitable spacer or linker (10), to the first member (6) of the binding pair (6/7) so as to form a first fusion protein. Also, the second binding member (7) of the binding pair (6/7) will usually, and preferably, be part of a second fusion protein that is different from the first fusion protein, which second fusion protein is also as further described herein. Said first fusion protein, said second fusion protein (in its various formats as described herein), nucleotide sequences and/or nucleic acids that encode the first or second fusion protein, and cells, cell lines or other host cells or host organisms that express (and in particularly suitably express, as described herein) or are capable of (suitably) expressing the first and/or the second fusion protein (and preferably both), as well as the various uses of the same as further described herein, form further aspects of the invention.

The binding pair (6/7) that is used in the arrangements that employ a chimeric GPCR of the invention will generally comprise at least two separate binding members (6) and (7), which are also referred to herein as the “first binding member” and the “second binding member”, respectively. The binding pair (6/7) and each member (6) and (7) thereof should be such that the binding pair (6/7) is capable of generating a detectable signal when the members (6) and (7) come into contact or in close proximity to each other. Such a detectable signal can for example be a luminescent signal, fluorescence signal or chemiluminescence signal.

In one specifically preferred aspect, when the methods described herein are performed in a suitable cell, the first member (6) and the second member (7) of the binding pair (6/7) are preferably both a polypeptide, protein, amino acid sequence or other chemical entity that can be obtained by suitably expressing, preferably in the cell that is used in the method of the invention, a nucleic acid or nucleotide sequence that encodes the same.

The first and second binding members can also be part of a suitable reporter assay, can be an enzyme-and-substrate combination, or any other pair of domain or units that can generate a detectable signal when they come into contact with, or close proximity to, each other, such as binding pairs that are commonly used in experimental study of protein-protein interactions. As mentioned, to reduce the level of baseline/background signal, it is preferred that the two members of the binding pair by themselves do not have a substantial binding affinity for each other.

Some preferred but non-limiting examples of suitable binding pairs are pGFP and the NanoBiT® system from Promega. The latter is especially preferred because the Large BiT and the small BiT that make up the NanoBiT® system by themselves have low affinity for each other.

The first binding member (6) can be fused in any suitable manner to the chimeric GPCR of the invention, as long as the resulting first fusion protein is such that it allows the first member (6) to come into contact with (or otherwise suitably in close proximity to) the second member (7) of the binding pair (6/7) when the second fusion protein formed by the second ligand (4) and the second member (7) binds to the chimeric GPCR of the invention via the second binding site (9) (i.e. to the intracellular binding site as defined herein). Also, preferably, first binding member (6) is fused or linked to the chimeric GPCR of the invention in a way that essentially does not affect, under the conditions used to perform the methods of the invention, the conformations and/or conformational changes that the chimeric GPCR of the invention can undergo.

Thus, generally, although it is not excluded in the invention that the first binding member (6) is fused or linked directly to the chimeric GPCR of the invention, it is generally preferred that the first binding member (6) is fused or linked to the chimeric GPCR via a suitable linker (10). The use of a flexible linker, for example with a total of between 5 and 50 amino acids, preferably between 10 and 30 amino acids, such as about 15 to 20 amino acids, is usually preferred. Suitable linkers will be clear to the skilled person and include GlySer linkers (for example a 15GS linker).

In the invention, the first and second binding members of the binding pair (6/7) will be present in (as defined herein) the same environment relative to the boundary layer (1), such that they can come into contact or close proximity to each other (in the manner as further described herein) and upon doing so can generate a detectable signal. In particular, as schematically shown in FIGS. 1, 2 and 3, the first and second binding members of the binding pair (6/7) will be present in (as defined herein) the same environment as the second binding site (9) (i.e. the intracellular binding site as defined herein) on the chimeric GPCR of the invention (again, relative to the boundary layer (1)), so as to allow first and second binding members of the binding pair (6/7) to come into contact when the second fusion protein binds to said binding site, i.e. either directly (as shown in FIG. 1) or indirectly (as shown in FIGS. 2 and 3). For this, the first binding member (6) will generally be attached, directly or via linker (10), to an amino acid residue/position in/on the chimeric GPCR of the invention that is exposed to the same environment as the second binding site (9). As further described herein, said environment (indicated as environment [B] in FIGS. 1 to 3) can for example be the intracellular environment (when the method of the invention is performed in cells) or the environment inside a vesicle or liposome.

In a preferred aspect of the invention, the first binding member (6) will be fused, directly or via the linker (10), to one end of the primary amino sequence of the chimeric GPCR of the invention. This may be the N-terminus or the C-terminus of the chimeric GPCR of the invention, again as long as in the final arrangement of the invention the first binding member (6) is on the same side of the boundary layer (1) as the second binding site (9). Accordingly, in the aspect of the invention that is performed in cells as further described herein, and where the second binding site (9) is exposed to the intracellular environment, the first member (6) may be fused to the end of the primary amino acid sequence that terminates in the intracellular environment (which, in the case of 7TMs, will usually be the C-terminal end).

The first fusion protein may be provided and produced using suitable techniques of protein chemistry and/or recombinant DNA technology known per se. Such techniques will be clear to the skilled person based on the further disclosure herein as well as the standard handbooks and other scientific references referred to herein. When the method of the invention is performed in cells (as further described herein), the first fusion protein is preferably provided by suitably expressing, in said cell, a nucleotide sequence and/or nucleic acid that encodes the first fusion protein. This can again be performed using suitable techniques of recombinant DNA technology known per se, and cells that suitably express or (are capable of suitably expressing) the first fusion protein form a further aspect of the invention.

As further described herein, in the arrangements of the invention, the second member (7) of the binding pair (6/7) will usually and preferably also form part of a fusion protein, which fusion protein will generally comprise said second binding unit which is fused or linked, either directly or via a suitable spacer or linker (11), to another ligand, protein, binding domain or binding unit, which ligand, protein, binding domain or binding unit is such that it can bind directly (as defined herein) or indirectly (as defined herein) to the chimeric GPCR of the invention. For this purpose, as further described herein, said ligand, protein, binding domain or binding unit may for example be the second ligand (resulting in an arrangement of the invention of the type that is schematically shown in FIG. 1, with (4) being the second ligand), be a binding domain or binding unit that can bind to the second ligand (resulting in an arrangement of the invention of the type that is schematically shown in FIG. 2, with (4) being the second ligand and (5) being the binding domain or binding unit binding to the second ligand), or be a binding domain or binding unit that can bind to a protein complex that can bind to the translayer protein (as schematically shown in FIG. 3, with (4) being the second ligand, (12) being said protein complex comprising the second ligand, and (5) being the binding domain or binding unit that binds to the protein complex).

In the second fusion protein, the second binding member (7) is most preferably linked to said other ligand, protein, binding domain or binding unit in a suitable manner that allows the second binding member (7) to come into contact with (or otherwise suitably in close proximity to) the first member (6) of the binding pair (6/7) when the second fusion protein binds directly or indirectly to the second binding site (9) (i.e. to the intracellular binding site as defined herein) on the chimeric GPCR of the invention. For this, the second binding member (7) may be fused or linked directly to said other ligand, protein, binding domain or binding unit, but preferably they are linked via a suitable linker (11), which is preferably a flexible linker, for example with a total of between 5 and 50 amino acids, preferably between and 30 amino acids, such as about 15 to 20 amino acids, is usually preferred. Suitable linkers will be clear to the skilled person and include GlySer linkers (for example a 15GS linker).

As mentioned herein, generally, the second ligand can be any ligand, protein, binding domain or binding unit is capable of binding to the translayer protein, i.e. via the binding site (9) (when the second ligand is part of the second fusion protein, it should most preferably also be such that it can be suitably included in the second fusion protein). Preferably, however, the second ligand is a conformation-inducing (as defined herein) binding domain or binding unit (and more preferably a conformation-inducing ISVD) that can bind to at least one of the ICLs of the chimeric GPCR of the invention.

Generally, in the invention (and irrespective of whether said binding site is bound directly or indirectly by the second fusion protein used in the arrangements of the invention), the binding site (9) can be a conformational epitope on the chimeric GPCR of the invention. More in particular, said binding site (9) can be a conformational epitope on the chimeric GPCR of the invention that changes it “shape” (i.e. the spatial arrangement of the domains, loops and/or amino acid residues that form the epitope) when the chimeric GPCR undergoes a conformational change, for example a conformational change from an inactive or less active state into an active, more active and/or functional state and/or a conformational change that occurs when a first ligand binds to the translayer protein.

Preferably, the binding site (9) and the second ligand are such that the affinity for the interaction between the binding site (9) and the second ligand (4) changes when the binding site (9) changes it shape because the chimeric GPCR undergoes a conformational shape. In particular, the binding site (9) and the second ligand may be such that the affinity for the interaction between the binding site (9) and the second ligand (4) increases when the chimeric GPCR undergoes a conformational change from an inactive or less active state into an active, more active, functional and/or druggable state and/or undergoes a conformational change that occurs when a first ligand (3) (and in particular a first ligand (3) that acts as an agonist in respect of the chimeric GPCR) binds to the chimeric GPCR.

In particular, the second ligand (4) and its interaction with the binding site (9) may be such that the second ligand (4) binds with higher affinity to the binding site (9) when the chimeric GPCR of the invention is an active, more active and/or functional state and/or such that the second ligand (4) binds with higher affinity to the binding site (9) when a first ligand (3) (and in particular a first ligand (3) that acts as an agonist with respect to the chimeric GPCR of the invention) binds to the chimeric GPCR. For example, the second ligand (4) and its interaction with the binding site (9) can be such that the affinity of the second ligand (4) for the chimeric GPCR of the invention increases 10 fold, such as 100 fold or more, when the chimeric GPCR undergoes such a conformational change, for example from an affinity in the micromolar range (i.e. more than 1000 nM) when the translayer protein is in an inactive, less active or ligand-free conformation to an affinity in the nanomolar range (i.e. less than 1000 nM, such as less than 100 nM) when the chimeric GPCR is in a functional, active or more active and/or ligand-bound conformation. For example, it is known that the affinity for the interaction between the G-protein and the G-protein binding site increases when a ligand (and in particular an agonist) binds to the extracellular binding site of the GPCR. Also, WO2012/007593, WO2012/007594, WO2012/75643, WO 2014/118297, WO2014/122183 and WO 2014/118297 describe VHH domains (ConfoBodies) that have higher affinity for a GPCR when the GPCR is in a functional, active or more active and/or ligand-bound conformation compared to when the GPCR is in an inactive, less active or ligand-free conformation (e.g. in the nanomolar range for a functional, active or ligand-bound conformation vs in the micromolar range for an inactive or ligand-free conformation). Such ConfoBodies can be used in the invention as a conformation-inducing ISVD, depending on the ICLs that are present in the chimeric GPCR of the invention.

When the second ligand is not a conformation-inducing binding domain or binding unit, the second ligand (4) will usually be a protein or a proteinaceous ligand. In the aspects of the invention that are performed in a suitable cell or cell line, the second ligand (4) may be a protein that is native to the cell or cell line used or may be a suitable (recombinant) protein that is expressed in the cell or cell line used. For example, when the second ligand (4) is not part of the second fusion protein, it can be a ligand of the “second” GPCR (i.e. the GPCR from which the ICls have been derived) that naturally occurs in said cell or cell line (for example, a G-protein that is natively expressed by the cell or cell line used). Alternatively, the second ligand may be a protein that is recombinantly expressed in the cell or cell line used, for example when said cell or cell line does not natively express a suitable ligand for the chimeric GPCR of the invention or when it is desired to use a ligand that is different from the ligand(s) that natively are expressed by said cell or cell line (for example, when it is desired to use an analog, derivative or ortholog of the natively expressed ligand, in which case the native expression of the natively expressed ligand may also be temporarily or constitutively suppressed or knocked-out in the cell or cell line used). When the second ligand (4) forms part of the second fusion protein, the second ligand will usually be expressed recombinantly as part of the second fusion protein.

As further described herein, the second ligand (4) can either be part of the second fusion protein or it can be separate from the second fusion protein. In either case (i.e. irrespective of whether the second ligand is part of the second fusion protein or not), the second ligand is preferably such that it is capable of binding to a conformational epitope on the chimeric GPCR of the invention (or such that it is part of a protein complex that binds directly to the chimeric GPCR or that is capable of binding directly to the chimeric GPCR). More preferably, the second ligand (and/or the protein complex that comprises the second ligand) is preferably such that it specifically binds to one or more functional, active and/or druggable conformations of the chimeric GPCR of the invention, such that it induces the formation of and/or stabilizes one or more functional, active and/or druggable conformations of the chimeric GPCR (and/or shifts the conformational equilibrium of the translayer protein towards one or more such conformations); and/or such that it induces the formation of and/or stabilizes a complex of the chimeric GPCR, the first ligand and the second ligand.

When the second ligand is part of the second fusion protein, it can be any ligand, binding domain, binding unit, peptide, protein or other chemical entity that can bind directly to the chimeric GPCR of the invention and that can suitably be included in the second fusion protein. Preferably, as further described herein, when it is part of the second fusion protein, the second ligand will be a suitable binding domain or binding unit, and in particular an immunoglobulin single variable domain. As mentioned herein, the second ligand is preferably a conformation-inducing binding domain or binding unit, and the use of a second fusion protein that comprises such a conformation-inducing binding domain or binding unit is also preferred.

When the second ligand is separate from the second fusion protein, it can be any ligand or protein that can bind directly to the chimeric GPCR and/or that can form part of a protein complex that can bind to the chimeric GPCR. For example, as further described herein, such a second ligand may be a naturally occurring ligand of the “second” GPCR from which the ICLs of the chimeric GPCR have been obtained, a semi-synthetic or synthetic analog or derivative of such a naturally occurring ligand or an ortholog of such a naturally occurring ligand. Also, when the second ligand is not part of the second fusion protein, the second fusion protein will comprise a binding domain or binding unit that can bind indirectly (as defined herein) to the chimeric GPCR, i.e. a binding domain or binding unit that can bind to the second ligand and/or to a protein complex that comprises the second ligand. Again, as also further described herein, such a binding domain or binding unit may in particular be an immunoglobulin single variable domain, such as a camelid-derived ISVD.

It will also be clear to the skilled person that, when the second ligand does not form part of the second fusion protein, that the binding domain or binding unit that is present in the second fusion protein and that can bind to the second ligand should essentially not interfere with the binding of the second ligand to the chimeric GPCR of the invention. For example, it is preferably such that it binds to a binding site or epitope on said second ligand that is distinct from the binding site on the second protein that binds to the chimeric GPCR (and preferably also sufficiently removed from the binding site on the second protein that binds to the chimeric GPCR so as to avoid any major steric hindrance).

When the second ligand (4) is a naturally occurring ligand of the “second” GPCR from which the ICLs have been derived, it may for example be a ligand that is involved in the signaling pathway or signaling transduction in which the translayer protein (2) is involved. For example, the second ligand (4) may be a naturally occurring ligand of the receptor, and in particular a naturally occurring intracellular ligand of the “second” GPCR, for example an intracellular ligand that binds to an intracellular binding site on the receptor when an extracellular ligand binds to an extracellular binding site on the receptor or, when the receptor has some degree of constitutive activity, that binds to an intracellular binding site of the receptor as part of the pathway that provides said constitutive activity. Suitable examples of such a natural ligand will be clear to the skilled person based on the disclosure herein, with a G-protein being a preferred example.

As further described herein, and in particular in aspects and embodiments of the invention that are performed using a cell or cell line, the second ligand (4) may also be part of a complex that comprises the second ligand (4) and optionally one or more further proteins. For example, when the second ligand is a G-protein or an analog or derivative of a G-protein, the second ligand may be part of a complex formed by said G-protein and optionally one or more further proteins. One preferred but non-limiting example of such a complex is the G-protein trimer comprising a G-alpha subunit, a G-beta subunit and a G-gamma subunit. Said complex may also comprise the translayer protein itself (e.g. the GPCR and the G-protein or the GPCR and the G-protein trimer). It will be clear to the skilled person that, when the second ligand forms part of such a complex, it is generally preferred that the second ligand does not form part of the second fusion protein. Instead, the second fusion protein will comprise a binding domain or binding unit that can bind to the second ligand or to said complex. For example, in case the second ligand forms part of a G-protein complex, the binding domain or binding unit in the second fusion protein can be a VHH domain that binds to said complex, for example to a subunit within said complex or to an interface between two or more of the said subunits. As mentioned herein, example of such a VHH domain is the VHH referred to as “CA4435” (SEQ ID NO:1 in WO2012/75643 and SEQ ID NO:22 herein).

The second ligand (4) may also be a synthetic or semi-synthetic analog or derivative of such a naturally occurring ligand, for example an analog or derivative with a primary amino acid sequence that differs from the primary amino acid sequence of the corresponding natural ligand by deletion, insertion and/or substitution of a limited number of amino acid residues or stretches of amino acid residues. Such analogs or derivatives may again be provided using suitable techniques of recombinant DNA technology known per se, which again in one aspect may involve expression in a suitable host or host cell of a nucleotide sequence or nucleic acid that encodes the analog or derivative (preferably, as part of the entire second fusion protein also including the second binding member (7) and any linker (11), if present). For example, the second ligand (4) may be an analog or derivative of G-protein (preferred), which again may have one or more amino acid differences (as defined herein) with the native sequence, provided that the analog or derivative still has sufficient affinity for the chimeric GPCR of the invention to allow the analog or derivative to be suitably used in the methods of the invention.

As mentioned, the second ligand is preferably a conformation-inducing binding domain or binding unit, and in particular a conformation-inducing ISVD as generally described in WO2012/007593, WO2012/007594, WO2012/75643, WO 2014/118297, WO2014/122183 and WO 2014/118297, which describe VHH domains (Confobodies) that are capable of stabilizing a GPCR in a desired conformation.

Also, WO2012/75643 discloses a number of VHH domains that can bind indirectly to a GPCR, i.e. by binding to a G protein or a G protein complex. Some preferred but non-limiting examples of these are the VHH referred to as “CA4435” (SEQ ID NO:1 in WO2012/75643 and SEQ ID NO:22 herein) which can bind to the G-protein complex and the VHH referred to as “CA4437” (SEQ ID NO:4 in WO2012/75643 and SEQ ID NO:23 herein) which can bind to the G-protein. Such VHH domains can be suitably included in the second fusion so as to provide a second fusion protein that can bind indirectly to a GPCR by binding to the G-protein or G-protein complex.

Generally, in the invention, the first binding member (6) and the second binding member (7) will come into close proximity to each other when the second fusion protein binds directly or indirectly (both as defined herein) to the chimeric GPCR of the invention. In particular, the first and second binding member will come into close proximity to each other when the second ligand (4) that is present in the second fusion protein binds directly to the chimeric GPCR of the invention or when the binding domain or binding unit (5) that is present in the second fusion protein binds indirectly to the chimeric GPCR of the invention, i.e. when said binding domain or binding unit (5) binds to the second ligand (4) or, in the case of the embodiment shown in FIG. 3, to the protein complex (12), which second ligand (4) or protein complex (12) in turn binds to or is bound by the chimeric GPCR of the invention. It will be clear to the skilled person that preferably, the first and second binding member should not by themselves have a high affinity for each other, so that their association (and the concomittent generation of the detectable signal) are driven mainly by the first and second binding member coming into each other's proximity because the second ligand binds (directly or indirectly) to the translayer protein, and essentially not or only by a lesser degree by the affinity between the first and second ligand (with the NanoBiT system from Promega being an example of such a suitable binding pair). However, it should also be noted that any such affinity between the first and second ligand will generally provide a baseline for the detectable signal which should essentially not interfere with the assay of the invention as the read-out of this assay primarily looks at any changes in the detectable signal for example upon adding the first ligand to an arrangement of the invention that does not yet comprise the first ligand (more generally it should also be noted that for some uses of the methods and arrangements of the invention, it may be preferable to have some level of baseline signal, as the read-out can then also comprise a decrease in signal compared to the baseline).

Thus, generally, in the invention, the detectable signal that is generated by the first and second binding members (or any change in said signal) will be proportional to the amount of second fusion protein that is bound directly or indirectly to the chimeric GPCR of the invention. This in turn will depend on the binding interaction between the second ligand (4) and the chimeric GPCR (and in particular, between the second ligand and one or more specific conformations that the chimeric GPCR can assume, such as a functional, active and/or druggable conformation) and/or on any changes to said binding interaction (and in particular on any changes to said binding interaction that are the result of a conformational change in the chimeric GPCR and/or a shift in the conformational equilibrium of the chimeric GPCR, for example due to the binding of the first ligand to the chimeric GPCR and/or the formation of a complex between the first ligand, the chimeric GPCR protein and the second ligand).

Based on this and the further disclosure herein, it will be clear to the skilled person that the methods and arrangements of the invention can be used to measure or determine one or more properties of the first ligand (and in particular, the properties of the first ligand that relate to, influence and/or determine the interaction between the first ligand and the chimeric GPCR of the invention, which as mentioned should be representative for the same properties with respect to the “first” GPCR from which the ECLs of the chimeric GPCRs have been derived).

More in particular, with respect to the first ligand, the methods and arrangements of the invention can be used to measure or determine the ability of the first ligand to bind to the chimeric GPCR, to effect a conformational change in the chimeric GPCR and/or to effect a change in the conformational equilibrium of the chimeric GPCR. For example, as further described herein, the methods and arrangements of the invention can be to measure or determine the ability of a given first ligand to act as an agonist, antagonist, inverse agonist, inhibitor or modulator (such as an allosteric) modulator of the chimeric GPCR and/or to screen for or identify small molecules, proteins or other compounds or chemical entities that act or can act as an agonist, antagonist, inverse agonist, inhibitor or modulator (such as an allosteric) modulator of the chimeric GPCR. In this respect, it will be clear to the skilled person based on the disclosure herein that when the methods and arrangements of the invention are to be used for such a purpose (i.e. for a purpose with respect to the first ligand), that then usually (and preferably) the other elements used in the arrangement of the invention (such as the second ligand and/or any binding domain or binding unit present in the second fusion protein) will be chosen such that they have known properties (i.e. that their properties relevant to their use in the methods and the arrangements of the invention are known and/or have been characterized) and/or such that they have already been validated for use in the methods and the arrangements of the invention. Also, as mentioned, the properties of the first ligand with respect to the chimeric GPCR of the invention are intended to be, and most preferably are, representative of the same properties of the first ligand with respect to the “first” GPCR from which the ECLs that are present in the chimeric GPCR of the invention have been derived.

In the invention, generally, the detectable signal will preferably be generated in response to, and more preferably also proportional to, a conformational change in the chimeric GPCR of the invention and/or a shift in the conformational equilibrium of the chimeric GPCR of the invention. As also further described herein, but again without being limited to any specific mechanism or explanation, said conformational change and/or shift in the conformational equilibrium of the chimeric GPCR of the invention may in turn be caused by a first ligand binding to the chimeric GPCR of the invention (or otherwise causing a conformational change in the chimeric GPCR) and/or by the formation of a complex of the first ligand, the chimeric GPCR of the invention and the second ligand (which second ligand may for example stabilize said complex or otherwise induce or promote the formation of said complex). Thus, more generally, in the invention, the detectable signal (or any change therein, as further described herein) will be generated in response to the presence of the first ligand in the first environment and/or in response to the first ligand binding to the chimeric GPCR of the invention (or otherwise causing a conformational change in the translayer protein and/or a shift in the conformational equilibrium of the chimeric GPCR of the invention).

Also, usually, and in particular when the methods and arrangements of the invention are used to test, optimize and/or validate a first ligand and/or to identify small molecules, proteins, ligands or other chemical entities that can act as an agonist, antagonist, inverse agonist, inhibitor or modulator (such as an allosteric) modulator of the chimeric GPCR of the invention (and so, of the first GPCR from which the ECLs of the chimeric GPCR has been derived), the detectable signal (or any change therein, as further described herein) will be proportional to the amount and/or concentration of the first ligand that is present in the first environment (and/or to which the chimeric GPCR is exposed) and/or to the affinity of the first ligand for the chimeric GPCR (e.g. in comparison to other ligands tested).

Thus, based on the description herein, it will be clear to the skilled person that in one aspect of the invention, the methods and arrangements described herein will be used to detect the presence of, and/or to determine the amount and/or concentration of, the first ligand in the first environment. The methods and arrangements described herein may also be used to measure the amount of signal that arises when different concentrations of the first ligand are present in the first environment, for example to establish a relationship between the amount/concentration of the first ligand in the first environment and the (level of and/or change in) the detectable signal. The methods and arrangements described herein may also be used to determine the affinity of the first ligand for the chimeric GPCR, for example by comparing the signal generated by one or more known concentrations of the first ligand in the first environment with signals generated in the same arrangement by known concentrations of other ligands with known affinity for the chimeric GPCR of the invention.

As further described herein, the methods and arrangements of the invention may also be used to determine whether a given (first) ligand is an agonist, antagonist, inverse agonist, inhibitor or modulator (such as an allosteric) modulator of the translayer protein.

It will also be clear to the skilled person that, when the methods and arrangements of the invention are being used to determine one or more characteristics of the first ligand, that the arrangement of the invention will usually first be set-up or otherwise established without the first ligand being present, and that then subsequently the arrangement will be contacted with the ligand (e.g. by adding the ligand to the first environment), after which the detectable signal (or any change therein) that results from the presence of the first ligand will be measured (and optionally compared to the signal without the presence of the first ligand and/or with one or more reference values). Thus, the arrangements described herein without the first ligand being present (for example, before the first ligand is added) form further aspects of the invention.

Another aspect of the invention is a method for providing an arrangement of the invention as described herein, which method comprises the step of adding a first ligand to an arrangement of the invention (as described herein) that does not (yet) comprise a first ligand. The arrangement thus obtained may then be used to measure or otherwise determine at least one property of the first ligand, and in particular a property of the first ligand that can be measured or otherwise determined using the arrangement of the invention.

As will be clear to the skilled person based on the disclosure herein, an arrangement of the invention without the first ligand being present (i.e. an arrangement of the invention that does not yet comprise a first ligand) will at least comprise the following elements:
a boundary layer that separates a first environment from a second environment;
a translayer protein;
a ligand for the translayer protein that is present in the second environment; and
a binding pair that consists of at least a first binding member and a second binding member, which binding pair is capable of generating a detectable signal;
   in which said translayer protein is a chimeric GPCR of the invention as described herein and in which elements are arranged with respect to each other (and where applicable operably linked to and/or associated with each other) in the manner as further described herein (i.e. essentially in the same way as described for the arrangements of the invention that comprise the first ligand). As described herein, ligand for the translayer protein (i.e. for the chimeric GPCR) is preferably a binding domain or binding unit that can bind to a binding site on said chimeric GPCR that comprises at least one of said intracellular loops (as described herein) and is more preferably a conformation-inducing binding domain or binding unit (also as described herein) and may in particular be an ISVD and more in particular a conformation-inducing ISVD.

In particular, an arrangement of the invention without the first ligand being present (i.e. an arrangement of the invention that does not yet comprise a first ligand) will at least comprise the following elements:
a boundary layer that separates a first environment from a second environment;
a binding pair that consists of at least a first binding member and a second binding member, which binding pair is capable of generating a detectable signal;
a translayer protein that is suitably fused or linked (either directly or via a suitable linker or spacer) to one of the binding members of said binding pair (i.e. so as to form a first fusion protein); and
a second ligand for the translayer protein that is present in the second environment;
   in which said translayer protein is a chimeric GPCR of the invention as described herein and in which elements are arranged with respect to each other (and where applicable operably linked to and/or associated with each other) in the manner as further described herein (i.e. essentially in the same way as described for the arrangements of the invention that comprise the first ligand). In particular, the second member of the binding pair may be part of a second fusion protein (which is different from the first fusion protein that comprises the translayer protein and the first binding member of the binding pair), which second fusion protein is as further described herein.

More in particular, an arrangement of the invention without the first ligand being present (i.e. an arrangement of the invention that does not yet comprise a first ligand) will at least comprise the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a second fusion protein comprising a protein that can bind directly or indirectly to the translayer protein and the other binding member of said binding pair, which second fusion protein is present in the second environment;
   in which said translayer protein is a chimeric GPCR of the invention as described herein and in which elements are arranged with respect to each other (and where applicable operably linked to and/or associated with each other) in the manner as further described herein (i.e. essentially in the same way as described for the arrangements of the invention that comprise the first ligand). Again, when the second ligand binds can bind directly to the chimeric GPCR, a binding domain or binding unit that can bind to a binding site on said chimeric GPCR that comprises at least one of said intracellular loops (as described herein) and is more preferably a conformation-inducing binding domain or binding unit (also as described herein) and may in particular be an ISVD and more in particular a conformation-inducing ISVD.

Other aspects, embodiment and preferences for such arrangements without the first ligand are as described herein for the arrangements of the invention with the first ligand, but then without the first ligand being present.

Generally, any such arrangement without the first ligand being present will become a corresponding arrangement with the first ligand once the first ligand is added as part of the methods described herein. Thus, another aspect of the invention is a method for providing an arrangement comprising a chimeric GPCR of the invention as described herein, which method comprises the step of adding a first ligand to an arrangement comprising a chimeric GPCR of the invention (as described herein) that does not (yet) comprise a first ligand. The arrangement thus obtained may then be used to measure or otherwise determine at least one property of the first ligand, and in particular a property of the first ligand that can be measured or otherwise determined using an arrangement that comprises a chimeric GPCR of the invention.

The invention also relates to a method of measuring or otherwise determining at least one property of a compound or ligand, which method comprises at least the steps of
adding said compound or ligand as a first ligand to an arrangement as described herein that comprises a chimeric GPCR of the invention that does not yet comprise a first ligand; and
measuring or otherwise determining at least one property of said compound or ligand, in which said property is a property that can be measured or otherwise determined using said arrangement.

In this aspect of the invention, said property is preferably a property that is representative for the ability of the compound or ligand to bind to and/or to modulate the chimeric GPCR (such as affinity), which in turn is representative for essentially the same property in respect of the first GPCR from which the ECLs (and preferably also essentially the TMs) have been obtained.

The invention also relates to a method of measuring or otherwise determining the ability of a compound or ligand to change the detectable signal that is generated by a binding pair that is present in an arrangement of the invention as further described herein, which method comprises at least the steps of
adding said compound or ligand as a first ligand to an arrangement comprising a chimeric GPCR of the invention that does not yet comprise a first ligand; and
determining whether adding said compound or ligand results in a change in the detectable signal that is generated by the binding pair used in said arrangement, and optionally measuring said change in said detectable signal.

Thus, in another aspect, the invention relates to a method that comprises at least the steps of:

a) providing an arrangement that at least comprises the following elements:
a boundary layer that separates a first environment from a second environment;
a translayer protein;
a ligand for the translayer protein that is present in the second environment; and
a binding pair that consists of at least a first binding member and a second binding member, which binding pair is capable of generating a detectable signal;
in which said translayer protein is a chimeric GPCR of the invention and in which said elements are arranged with respect to each other (and where applicable operably linked to and/or associated with each other) in the manner as further described herein;
   and;
b) adding a first ligand to the first environment.
   which method preferably further comprises the step of
c) measuring the signal that is generated by the binding pair and/or measuring the change in the signal that is generated by the binding pair.

In a more specific aspect, the invention relates to a method that comprises at least the steps of:
a) providing an arrangement that at least comprises the following elements:
   a boundary layer that separates a first environment from a second environment;
   a binding pair that consists of at least a first binding member and a second binding member, which binding pair is capable of generating a detectable signal;
   a translayer protein that is suitably fused or linked (either directly or via a suitable linker or spacer) to one of the binding members of said binding pair (i.e. so as to form a first fusion protein); and
   a second ligand for the translayer protein that is present in the second environment;
   in which said translayer protein is a chimeric GPCR of the invention and in which said elements are arranged with respect to each other (and where applicable operably linked to and/or associated with each other) in the manner as further described herein;
      and;
b) adding a first ligand to the first environment.
   which method preferably further comprises the step of:
c) measuring the signal that is generated by the binding pair and/or measuring the change in the signal that is generated by the binding pair.

In another specific aspect, the invention relates to a method that comprises at least the steps of:
a) providing an arrangement that at least comprises the following elements:
   a boundary layer that separates a first environment from a second environment;
   a binding pair that consists of at least a first binding member and a second binding member, which binding pair is capable of generating a detectable signal;
   a first fusion protein comprising a translayer protein and one of the binding members of said binding pair (i.e. such that said member of the binding pair is present in the second environment);
   a second fusion protein comprising a protein that can bind directly or indirectly to the translayer protein and the other binding member of said binding pair, which second fusion protein is present in the second environment;
   in which said translayer protein is a chimeric GPCR of the invention and in which said elements are arranged with respect to each other (and where applicable operably linked to and/or associated with each other) in the manner as further described herein;
      and;
b) adding a first ligand to the first environment.
   which method preferably further comprises the step of:
c) measuring the signal that is generated by the binding pair and/or measuring the change in the signal that is generated by the binding pair.

As further described herein, in this aspect of the invention, said first ligand can be any desired and/or suitable compound or ligand, including but not limited to small molecules, small peptides, biological molecules or other chemical entities. It will also be clear to the skilled person that the method according to this aspect (and the other methods of the invention) can be used to measure or otherwise determine at least one property of the compound or ligand that is added to the arrangement as the first ligand, and in particular to measure or otherwise determine the ability of said compound or ligand to give rise to a change in the detectable signal that is generated by the binding pair, the ability of said compound or ligand to bind to the chimeric GPCR of the invention, the ability of said compound or ligand to effect a conformational change in the a chimeric GPCR of the invention, and/or the ability of said compound or ligand to modulate (as defined herein) the a chimeric GPCR of the invention. Again, said ability or abilities of the first ligand towards the chimeric GPCR of the invention is preferably representative for the corresponding ability or abilities of the first ligand in respect of the first GPCR from which the ECLs that are present in the chimeric GPCR of the invention have been derived. Also, by having the ability to modulate the first GPCR, the first ligand may also have the ability of modulate the signaling pathway(s) and/or biological mechanism(s) in which the first GPCR is involved. In particular, said methods can be used to determine whether such a compound or ligand is or can act as an agonist, antagonist, inverse agonist, inhibitor or modulator (such as an allosteric modulator) of the chimeric GPCR of the invention, of the first GPCR and/or the signaling pathway(s) and/or biological mechanism(s) in which the first GPCR is involved. Also, the methods and arrangements of the invention can be used to identify and/or screen for compounds or ligands that have the ability to give rise to a change in the detectable signal that is generated by the binding pair, the ability to bind to the chimeric GPCR of the invention, the ability to effect a conformational change in the chimeric GPCR of the invention (and thereby, in the first GPCR), the ability to modulate the chimeric GPCR of the invention and the first GPCR and/or the signaling pathway(s) and/or biological mechanism(s) in which the first GPCR is involved, and/or the ability to act as an agonist, antagonist, inverse agonist, inhibitor and/or modulator (such as an allosteric modulator) of the chimeric GPCR of the invention (and thereby, of the first GPCR), and such uses of the methods and arrangements described herein form further aspects of the invention.

As described herein, in one specific aspect of the invention, the methods of the invention are performed using a suitable cell or cell line in which all of the elements of an arrangement comprising a chimeric GPCR of the invention are suitably present and arranged so as to provide an operable arrangement of the invention. Such a cell or cell line will suitably comprise the chimeric GPCR in its cell wall or cell membrane, i.e. such that the chimeric GPCR is present in and spans the cell wall or cell membrane of the cell such that at least one part of the amino acid sequence of the chimeric GPCR (and in particular at least one of the ECLs, and preferably all of the ECLs) extends out (as defined herein) into the extracellular environment and at least one of other part of the amino acid sequence of the chimeric GPCR (and in particular at least one of the ECLs, and preferably all of the ECLs) extends out (as defined herein) into the intracellular environment. Also, preferably and as further described herein, the chimeric GPCR will form part of the first fusion protein as described herein and the arrangement will also comprise a second fusion protein as described herein. More preferably, the extracellular environment will be the “first environment” (i.e. the environment in which the first ligand (3) is present or to which the first ligand (3) is added) and the intracellular environment will be the “second environment” (i.e. the environment in which the binding pair (6/7) and the second fusion protein are present).

Thus, in a further aspect, the invention relates to a method or arrangement as described herein, in which the boundary layer (2) is the wall or the membrane of cell.

As also described herein, when the methods of the invention are performed in cells or in a suitable cell line, the cell or cell line used is preferably such that it suitably expresses one or more, and preferably all, of the following elements of the arrangement of the invention:
the first fusion protein comprising the chimeric GPCR of the invention and the first binding member (6);
the second fusion protein comprising the second binding member (7) and a protein that can bind directly or indirectly (as defined herein) to the translayer protein (2);
   and/or
when the second fusion protein binds indirectly to the chimeric GPCR, the second ligand (4) and/or the proteins that make up the protein complex (12).

In the context of a cell or cell line that expresses one or more elements of an arrangement of the invention, and more generally in the context of the present description and claims, with the term “suitably expresses” is meant that the cell or cell line expresses or is capable of expressing (i.e. under the conditions used for performing the methods of the invention) a nucleotide sequence or nucleic acid that encodes said element such that, when such element is expressed, it is capable of functioning as an operable part of the arrangement of the invention. For example, with respect to the chimeric GPCR of the invention, this means that the chimeric GPCR is expressed as part of the first fusion protein such that the chimeric GPCR becomes suitably anchored or otherwise incorporated into the cell wall or cell membrane of the cell such that it spans the cell wall or cell membrane with at least one part of the amino acid sequence of the chimeric GPCR (and in particular at least one of the ECLs, and preferably all of the ECLs) extending out (as defined herein) into the extracellular environment and at least one of other part of the amino acid sequence of the chimeric GPCR (and in particular at least one of the ECLs, and preferably all of the ECLs) extending out (as defined herein) into the intracellular environment. With respect to the first and second fusion protein, “suitably expresses” means that the first and second fusion protein are expressed such (and most preferably expressed in the intracellular environment such) that the first and second binding members of the binding pair (6/7) can come into contact or in close proximity to each other when the second fusion protein binds directly or indirectly to the chimeric GPCR of the invention, in the manner as further described herein.

Any suitable expression of each such element of an arrangement of the invention can be transient or constitutive expression, as long as all the required elements of the arrangement of the invention are suitably and operably present in sufficient amounts at the point in time when the cell is to be used for performing the method of the invention.

In one aspect of the invention, in case of an embodiment of the invention in which the second fusion protein binds indirectly to the chimeric GPCR of the invention (i.e. where the second ligand (4) is not part of the second fusion protein), the cell or cell line used is preferably such that it natively expresses the second ligand (4) and/or the proteins that make up the protein complex (12). For example and without limitation, in this aspect of the invention, the second ligand (4) may be a G protein that is natively expressed by the cell or cell line used and/or the protein complex (12) may be a G-protein trimer comprising a G-alpha subunit, a G-beta subunit and a G-gamma subunit that are natively expressed by the cell or cell line used. More generally, in these aspects of the invention, the cell or cell line used may be a cell or cell line that natively expresses one or more natural ligands (and in particular intracellular ligands) of the chimeric GPCR of the invention and/or that natively expresses one or more ligands that can function as a second ligand for the chimeric GPCR of the invention.

The cell or cell line can be any cell or cell line that suitable for use in the methods and arrangements of the invention, including but not limited to mammalian cells and insect cells. Some preferred but non-limiting examples are human cell lines such as HEK 293 T.

Suitable techniques for transiently or stably expressing a desired protein in such a cell or cell line such that the chimeric GPCR of the invention becomes suitably anchored into the cell wall or cell membrane of said cells will be clear to the skilled person and for example include techniques involving the use of a suitable transfection reagent such as X-tremeGENE™ from SigmaAldrich or polyethylenimine (PEI).

When the invention is performed using a cell or cell line that suitably expresses one or more elements of an arrangement of the invention, the method of the invention will generally also include a step of cultivating or maintaining said cell under conditions such that said cell or cell line suitably expresses said elements.

Thus, in another aspect, the invention relates to a cell or cell line that comprises a fusion protein, said fusion protein comprising a (as described herein) that is fused, directly or via a suitable linker, to a binding domain or binding unit that is a first binding member of a binding pair, said binding pair comprising at least said binding domain or binding unit as a first binding member and a further binding domain or binding unit as a second binding member, in which said first and second binding members of said binding pair are such that they are capable of generating a detectable signal when they come into contact with each other or into close proximity to each other. The invention also relates to a cell or cell line that expresses or is capable of expressing (i.e. under suitable conditions) such a fusion protein.

Such a cell or cell line can be as further described herein, and is preferably such that it expresses or is capable of expressing said fusion protein in such a way that the chimeric GPCR of the invention becomes incorporated into the cell wall or cell membrane of the cell or cell line and spans said cell wall or cell membrane, more preferably such that that at least one part of the amino acid sequence of the chimeric GPCR (and in particular at least one of the ECLs, and preferably all of the ECLs) extends out (as defined herein) into the extracellular environment and at least one of other part of the amino acid sequence of the chimeric GPCR (and in particular at least one of the ECLs, and preferably all of the ECLs) extends out (as defined herein) into the intracellular environment. More preferably, in said cell or cell line, the first binding member of the binding pair is present in (as defined herein) the intracellular environment of the cell and/or the cell or cell line is such that it expresses or is capable of expressing the fusion protein such that, upon such expression, the first binding member is present in (as defined herein) the intracellular environment of the cell.

In another aspect, the invention relates to a cell or cell line that comprises a fusion protein, said fusion protein comprising a protein that can bind (directly or indirectly, as described herein) to a chimeric GPCR of the invention, which protein is fused, directly or via a suitable linker, to a binding domain or binding unit that is a binding member of a binding pair, said binding pair comprising at least a first binding member and said binding domain or binding unit as a second binding member, in which said first and second binding members of said binding pair are such that they are capable of generating a detectable signal when they come into contact with each other or into close proximity to each other. The invention also relates to a cell or cell line that expresses or is capable of expressing (i.e. under suitable conditions) such a fusion protein.

The protein that is present in said fusion protein and that can bind to the chimeric GPCR of the invention is preferably as further described herein for the protein that can be present in the second fusion protein. Also, the members of the binding pair and any linkers used can be as further described herein. As also described herein, said protein can bind directly (as described herein) or indirectly (as described herein) to the chimeric GPCR of the invention.

As described herein, when the protein that is present in said fusion protein binds directly to the chimeric GPCR of the invention, it is preferably such that it specifically binds to one or more functional, active and/or druggable conformations of the chimeric GPCR of the invention, such that it induces the formation of and/or stabilizes one or more functional, active and/or druggable conformations of the chimeric GPCR (and/or shifts the conformational equilibrium of the translayer protein towards one or more such conformations); and/or such that it induces the formation of and/or stabilizes a complex of said protein, the chimeric GPCR and a further ligand of the chimeric GPCR (all as further described herein). Also, when the protein that is present in said fusion protein binds directly to the chimeric GPCR, the protein is preferably such that it can bind to an intracellular binding site on the chimeric GPCR. Said intracellular binding site on the chimeric GPCR can be a binding site that corresponds to the native intracellular binding site on the second GPCR from which the ICLs on the chimeric GPCR have been derived.

Also, when the protein that is present in said fusion protein binds directly to the chimeric GPCR, it is preferably a VHH domain or a binding domain or binding unit that is derived from a VHH domain, and in particular a ConfoBody (as described herein).

As also described herein, when the protein that is present in said fusion protein binds indirectly to the chimeric GPCR of the invention, it is preferably such that is can bind to a ligand that can bind to the chimeric GPCR of the invention. Said ligand can be as described herein for the “second ligand” when said second ligand does not form part of the second fusion protein. Again, said ligand is preferably such that it specifically binds to one or more functional, active and/or druggable conformations of the chimeric GPCR, such that it induces the formation of and/or stabilizes one or more functional, active and/or druggable conformations of the chimeric GPCR (and/or shifts the conformational equilibrium of the chimeric GPCR towards one or more such conformations); and/or such that it induces the formation of and/or stabilizes a complex of said ligand, the chimeric GPCR and a further ligand of the chimeric GPCR (all as further described herein). Also, said ligand is preferably such that it can bind to an intracellular binding site on the chimeric GPCR. Also, as described herein, said ligand can also be part of a protein complex that can bind to the chimeric GPCR (i.e. to an intracellular binding site on the chimeric GPCR), in which case the protein that is present in the fusion protein can also bind to said protein complex.

Also, when the protein that is present in said fusion protein binds indirectly to the, it is preferably a VHH domain or a binding domain or binding unit that is derived from a VHH domain. Also, in a preferred aspect, when the protein that is present in said fusion protein binds indirectly to the chimeric GPCR, the ligand binding to the GPCR is a G-protein and the protein that is present in said fusion protein is capable of specifically binding to said G-protein or to a G-protein complex such as a G-protein trimer that comprises a G-alpha subunit, a G-beta subunit and a G-gamma subunit). Also, said G-protein may be native to the cell or cell line used or may be a suitable analog or derivative (as described herein, and recombinantly expressed in said cell or cell line) of a natural G-protein or a suitable ortholog of the G-protein that is native to the cell or cell line used (again, recombinantly expressed in the cell or cell line used).

Irrespective of whether the protein that is present in said fusion protein binds directly or indirectly to the chimeric GPCR, the cell or cell line is preferably such that it expresses or is capable of expressing said fusion protein in the intracellular environment. Another aspect of the invention relates to such a cell or cell line that comprises such fusion protein in its intracellular environment.

In another aspect, the invention relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to said chimeric GPCR, which protein is fused, directly or via a suitable linker, to the second binding member of said binding pair.

The invention also relates to a cell or cell line that expresses or is capable of expressing (i.e. under suitable conditions) such first and second fusion proteins.

The invention in particular relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to said chimeric GPCR, which protein is fused, directly or via a suitable linker, to the second binding member of said binding pair; and
the first and second binding members of the binding pair can come into contact or in close proximity to each other when the second fusion protein binds (directly or indirectly, as described herein) to the translayer protein that forms part of the first fusion protein.

The invention also relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to said chimeric GPCR, which protein is fused, directly or via a suitable linker, to the second binding member of said binding pair; and
the first and second binding members of the binding pair are present in (as defined herein) the intracellular environment of the cell.

The invention further relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR (as described herein) that is fused, directly or via a suitable linker, to said first binding member of the binding pair; and
said second fusion protein comprises a protein that can bind (directly or indirectly, as described herein) to said chimeric GPCR, which protein is fused, directly or via a suitable linker, to the second binding member of said binding pair; and
said cell or cell line is capable of generating a detectable signal (and in particular, a detectable signal that is generated by the first and second binding members of the binding pair) when the second fusion protein binds (directly or indirectly, as described herein) to the chimeric GPCR.

The invention further relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR (as described herein) that is fused, directly or via a suitable linker, to said first binding member of the binding pair; and
said second fusion protein comprises a protein that can bind (directly or indirectly, as described herein) to said chimeric GPCR, which protein is fused, directly or via a suitable linker, to the second binding member of said binding pair; and
said cell or cell line gives rise to a detectable signal and/or to a change in a detectable signal (and in particular, to a detectable signal that is generated by the first and second binding members of the binding pair and/or to a change in such a signal) when a ligand for the chimeric GPCR that is present in the extracellular environment binds to the chimeric GPCR.

In a particular aspect, the invention relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR (as described herein) that is fused, directly or via a suitable linker, to said first binding member of the binding pair; and
said second fusion protein comprises a protein that can bind (directly or indirectly, as described herein) to said chimeric GPCR, which protein is fused, directly or via a suitable linker, to the second binding member of said binding pair; and
said cell or cell line gives rise to a detectable signal and/or to a change in a detectable signal (and in particular, to a detectable signal that is generated by the first and second binding members of the binding pair and/or to a change in such a signal) when an agonist for the chimeric GPCR that is present in the extracellular environment binds to the chimeric GPCR.

Again, such cells or cell lines that comprise or express such first and second fusion proteins can be as further described herein, and are preferably such that they expresses or are capable of expressing said first fusion protein in such a way that the chimeric GPCR becomes incorporated into the cell wall or cell membrane of the cell or cell line and spans said cell wall or cell membrane, more preferably such that at least one part of the amino acid sequence of the chimeric GPCR (and in particular at least one of the ECLs, and preferably all of the ECLs) extends out (as defined herein) into the extracellular environment and at least one of other part of the amino acid sequence of the chimeric GPCR (and in particular at least one of the ECLs, and preferably all of the ECLs) extends out (as defined herein) into the intracellular environment.

Said cells or cell lines are also preferably such that they expresses or are capable of expressing the first and second fusion protein such that, upon such expression, the first and second binding members of the binding pair can come into contact or in close proximity to each other when the second fusion protein binds (directly or indirectly, as described herein) to the chimeric GPCR that forms part of the first fusion protein. It will be clear to the skilled person that this generally means that such cells or cell lines will express the first and second fusion proteins in such a way that, upon such expression, the first and second binding members of the binding pair will be present in (as defined herein) the same environment relative to the wall or membrane of the cell. Preferably, the cells or cell lines are such that they express or are capable of expressing the first and second fusion protein such that, upon such expression, the first and second binding members of the binding pair will both be present in (as defined herein) the intracellular environment of the cell. This also generally means that the cells or cell lines are preferably such that they expresses or are capable of expressing the second fusion protein in their intracellular environment.

Again, in the aspects of the invention that relate to cells or cell lines that express or are capable of expressing such a first and second fusion protein, the chimeric GPCR, the protein that can bind directly or indirectly to the chimeric GPCR, the members of the binding pair and any linkers used can all be as further described herein.

In further aspects, the invention also relates to methods, and in particular assay methods or screening methods, that involve the use of the cells or cell lines described herein. As further described herein, such assay and screening methods can in particular be used to identify compounds and other chemical entities that bind to (and in particular specifically bind to) the chimeric GPCR (and thereby, to the first GPCR from which the ECLs that are present in the chimeric GPCR can be derived), that can modulate the chimeric GPCR and the first GPCR and/or that modulate the signaling, signaling pathway and/or biological or physiological activitie(s) in which the first GPCR, its signaling and/or its signaling pathway is involved. As such, the cells and cell lines described herein can be used in methods to identify compounds or other chemical entities that can act as an agonist, antagonist, inverse agonist, inhibitor or modulator (such as an allosteric) modulator of the chimeric GPCR and the first GPCR.

The invention also relates to uses of the cells or cell lines described herein, in particular in assay and screening methods and techniques. Such methods and uses can again be as further described herein for methods and uses of the arrangements of the invention, and will generally also include a step of cultivating or maintaining said cell under conditions such that said cell or cell line suitably expresses the desired fusion protein or proteins.

Again, in all these aspects, such cells, cell lines and uses thereof are preferably as further described herein.

In another aspect of the invention, the methods of the invention are performed using a suitable liposome or vesicle in which all of the elements of an arrangement of the invention are suitably present and arranged so as to provide an operable arrangement of the invention. Such a liposome or vesicle will suitably comprise the translayer protein (2) in its wall or membrane, i.e. such that the translayer protein (2) is present in and spans the wall or membrane of the liposome or vesicle such that at least one part of the amino acid sequence of the chimeric GPCR (and in particular at least one of the ECLs, and preferably all of the ECLs) extends out (as defined herein) into the environment outside of the liposome or vesicle and at least one of other part of the amino acid sequence of the chimeric GPCR (and in particular at least one of the ECLs, and preferably all of the ECLs) extends out (as defined herein) into the environment inside the liposome or vesicle. Also, preferably and as further described herein, in aspects of the invention that are performed in a liposome or vesicle, the environment outside of the liposome or vesicle will be the “first environment” (i.e. the environment in which the first ligand (3) is present or to which the first ligand (3) is added) and the environment inside the liposome or vesicle will be the “second environment” (i.e. the environment in which the binding pair (6/7) and the second fusion protein are present).

Thus, in a further aspect, the invention relates to a method or arrangement as described herein, in which the boundary layer (2) is the wall or the membrane of a liposome or other (suitable) vesicle.

As also described herein, when the methods of the invention are performed in a liposome or vesicle, the liposome or vesicle is preferably such that it suitably contains (i.e. in such a manner as to provide an operable arrangement of the invention) the following elements of the arrangement of the invention:
the first fusion protein comprising a and the first binding member (6);
the second fusion protein comprising the second binding member (7) and a protein that can bind directly or indirectly (as defined herein) to the chimeric GPCR;
   and/or
when the second fusion protein binds indirectly to the chimeric GPCR, the second ligand (4) and/or the proteins that make up the protein complex (12)

Liposomes or vesicles that contain said elements can generally be provided by forming the liposomes or vesicles in the presence of the relevant elements of the arrangement of the invention, such that said elements are suitably incorporated into the liposomes of vesicles. This can generally be performed by methods and techniques known per se for forming liposomes or vesicles, preferably in a suitable aqueous buffer or another suitably aqueous medium. Such methods may also comprise a step of separating liposome or vesicles in which the elements of the desired arrangement of the invention are suitably and operably included from vesicles or liposomes that do not contain all the required elements of the arrangements and/or in which the elements do not form an operable arrangement of the invention. The elements of the arrangements that are incorporated into the liposome of vesicle can be provided in a manner known per se, for example by recombinant expression is a suitable host cell or host organism followed by isolating and purifying the expressed elements thus obtained.

Generally, in the aspects of the invention that are performed in liposomes or vesicles, where the second ligand does not form part of the second fusion protein, a sufficient amount of the second ligand should also be provided and suitably included into the vesicle or liposome.

The liposome or vesicle can be any liposome or vesicle that suitable for use in the methods and arrangements of the invention, including but not limited to liposomes based on 1,2-dioleoyl-sn-glycero-3-phosphocholine (DOPC), dioleoylphosphatidylethanolamine (DOPE) or 1-palmitoyl-2-oleoyl-sn-glycero-3-phosphocholine (POPC). The liposomes and vesicles can also be liposomes or vesicles that contain and/or are based on (e.g. reconstituted from) one or more membrane fractions obtained from cells that express the desired element(s) of the arrangement of the invention.

Thus, in another aspect, the invention relates to a liposome or vesicle that comprises a fusion protein, said fusion protein comprising a chimeric GPCR of the invention (as described herein) that is fused, directly or via a suitable linker, to a binding domain or binding unit that is a first binding member of a binding pair, said binding pair comprising at least said binding domain or binding unit as a first binding member and a further binding domain or binding unit as a second binding member, in which said first and second binding members of said binding pair are such that they are capable of generating a detectable signal when they come into contact with each other or into close proximity to each other. The invention also relates to method for providing such a liposome or vesicle which method comprises at least the step of incorporating such a fusion protein into a liposome or vesicle and/or of forming a liposome or vesicle in the presence of said fusion protein.

As further described herein, said liposome or vesicle is preferably such that the chimeric GPCR is anchored or otherwise suitably incorporated into the wall or membrane of the liposome or vesicle and spans said wall or membrane, more preferably such that at least one part of the amino acid sequence of the chimeric GPCR (and in particular at least one of the ECLs, and preferably all of the ECLs) extends out (as defined herein) into the environment outside of the liposome or vesicle and at least one of other part of the amino acid sequence of the chimeric GPCR (and in particular at least one of the ECLs, and preferably all of the ECLs) extends out (as defined herein) into the environment inside the liposome or vesicle. More preferably, the first binding member of the binding pair is present in (as defined herein) the environment inside liposome or vesicle,

In another aspect, the invention relates to a liposome or vesicle that comprises a fusion protein, said fusion protein comprising a protein that can bind (directly or indirectly, as described herein) to a chimeric GPCR of the invention, which protein is fused, directly or via a suitable linker, to a binding domain or binding unit that is a binding member of a binding pair, said binding pair comprising at least a first binding member and said binding domain or binding unit as a second binding member, in which said first and second binding members of said binding pair are such that they are capable of generating a detectable signal when they come into contact with each other or into close proximity to each other. The invention also relates to method for providing such a liposome or vesicle which method comprises at least the step of incorporating such a fusion protein into a liposome or vesicle and/or of forming a liposome or vesicle in the presence of said fusion protein.

The protein that is present in said fusion protein and that can bind to the chimeric GPCR of the invention is preferably as further described herein for the protein that can be present in the second fusion protein. Also, the members of the binding pair and any linkers used can be as further described herein. As also described herein, said protein can bind directly (as described herein) or indirectly (as described herein) to the chimeric GPCR of the invention.

As described herein, when the protein that is present in said fusion protein binds directly to the chimeric GPCR of the invention, it is preferably such that it specifically binds to one or more functional, active and/or druggable conformations of the chimeric GPCR, such that it induces the formation of and/or stabilizes one or more functional, active and/or druggable conformations of the chimeric GPCR (and/or shifts the conformational equilibrium of the chimeric GPCR towards one or more such conformations); and/or such that it induces the formation of and/or stabilizes a complex of said protein, the chimeric GPCR and a further ligand of the translayer protein (all as further described herein). Also, when the protein that is present in said fusion protein binds directly to the chimeric GPCR, the protein is preferably such that it can bind to a binding site on the chimeric GPCR that corresponds to an intracellular binding site on the second GPCR (i.e. the one from which the ICLs in the chimeric GPCR of the invention have been obtained) when said second GPCR is in its native environment. When the methods of the invention are performed using cells or liposomes, said binding site on the chimeric GPCR is also preferably present in (as defined herein) the intracellular environment of the cell or the environment in said liposome or vesicle, respectively.

Also, when the protein that is present in said fusion protein binds directly to the chimeric GPCR of the invention, it is preferably a VHH domain or a binding domain or binding unit that is derived from a VHH domain, and in particular a ConfoBody (as described herein).

As also described herein, when the protein that is present in said fusion protein binds indirectly to the translayer protein (i.e. to the chimeric GPCR of the invention), it is preferably such that it can bind to a ligand that can bind to the translayer protein. Said ligand can be as described herein for the “second ligand” when said second ligand does not form part of the second fusion protein. Again, said ligand is preferably such that it specifically binds to one or more functional, active and/or druggable conformations of the translayer protein, such that it induces the formation of and/or stabilizes one or more functional, active and/or druggable conformations of the translayer protein (and/or shifts the conformational equilibrium of the translayer protein towards one or more such conformations); and/or such that it induces the formation of and/or stabilizes a complex of said ligand, the translayer protein and a further ligand of the translayer protein (all as further described herein). Also, said ligand is preferably such that it can bind to a binding site on the chimeric GPCR that corresponds to an intracellular binding site on the second GPCR from which the ICLs have been derived (i.e. when said second GPCR is in its native environment). Also, as described herein, said ligand can also be part of a protein complex that can bind to the translayer protein, in which case the protein that is present in the fusion protein can also bind to said protein complex.

Also, when the protein that is present in said fusion protein binds indirectly to the translayer protein (i.e. to the chimeric GPCR of the invention), it is preferably a VHH domain or a binding domain or binding unit that is derived from a VHH domain. Also, in a preferred aspect, when the protein that is present in said fusion protein binds indirectly to the translayer protein, and said translayer protein is a GPCR, the ligand binding to the GPCR is a G-protein and the protein that is present in said fusion protein is capable of specifically binding to said G-protein or to a G-protein complex such as a G-protein trimer that comprises a G-alpha subunit, a G-beta subunit and a G-gamma subunit).

Irrespective of whether the protein that is present in said fusion protein binds directly or indirectly to the chimeric GPCR of the invention, said fusion protein is preferably present in (as defined herein) the environment inside the liposome or vesicle. Also, when the second ligand does not form part of said fusion protein, the environment inside the liposome or vesicle will also contain a suitable amount of the second ligand.

In another aspect, the invention relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to the chimeric GPCR of the invention, which protein is fused, directly or via a suitable linker, to the second binding member of said binding pair.

The invention also relates to method for providing such a liposome or vesicle which method comprises at least the step of incorporating said fusion proteins into a liposome or vesicle and/or of forming a liposome or vesicle in the presence of said fusion proteins.

The invention in particular relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to said chimeric GPCR of the invention, which protein is fused, directly or via a suitable linker, to the second binding member of said binding pair; and
the first and second binding members of the binding pair can come into contact or in close proximity to each other when the second fusion protein binds (directly or indirectly, as described herein) to the chimeric GPCR of the invention that forms part of the first fusion protein.

Again, the invention also relates to method for providing such a liposome or vesicle which method comprises at least the step of incorporating said fusion proteins into a liposome or vesicle and/or of forming a liposome or vesicle in the presence of said fusion proteins.

The invention also relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to said chimeric GPCR of the invention, which protein is fused, directly or via a suitable linker, to the second binding member of said binding pair; and
the first and second binding members of the binding pair are present in (as defined herein) the environment inside the liposome or vesicle.

Again, the invention also relates to method for providing such a liposome or vesicle which method comprises at least the step of incorporating said fusion proteins into a liposome or vesicle and/or of forming a liposome or vesicle in the presence of said fusion proteins.

The invention further relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to said chimeric GPCR of the invention, which protein is fused, directly or via a suitable linker, to the second binding member of said binding pair; and
said liposome or vesicle is capable of generating a detectable signal (and in particular, a detectable signal that is generated by the first and second binding members of the binding pair) when the second fusion protein binds (directly or indirectly, as described herein) to the chimeric GPCR of the invention that forms part of the first fusion protein.

Again, the invention also relates to method for providing such a liposome or vesicle which method comprises at least the step of incorporating said fusion proteins into a liposome or vesicle and/or of forming a liposome or vesicle in the presence of said fusion proteins.

The invention further relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to said chimeric GPCR of the invention, which protein is fused, directly or via a suitable linker, to the second binding member of said binding pair; and
said liposome or vesicle gives rise to a detectable signal and/or to a change in a detectable signal (and in particular, to a detectable signal that is generated by the first and second binding members of the binding pair and/or to a change in such a signal) when a ligand for the chimeric GPCR of the invention that is present in the environment outside of the liposome or vesicle binds to the chimeric GPCR of the invention.

Again, the invention also relates to method for providing such a liposome or vesicle which method comprises at least the step of incorporating said fusion proteins into a liposome or vesicle and/or of forming a liposome or vesicle in the presence of said fusion proteins.

In a particular aspect, the invention relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a chimeric GPCR of the invention (as described herein) that is fused, directly or via a suitable linker, to said first binding member of the binding pair; and
said second fusion protein comprises a protein that can bind (directly or indirectly, as described herein) to said chimeric GPCR of the invention, which protein is fused, directly or via a suitable linker, to the second binding member of said binding pair; and
said liposome or vesicle gives rise to a detectable signal and/or to a change in a detectable signal (and in particular, to a detectable signal that is generated by the first and second binding members of the binding pair and/or to a change in such a signal) when an agonist for the chimeric GPCR of the invention that is present in the environment outside of the liposome or vesicle binds to the chimeric GPCR of the invention.

Again, the invention also relates to method for providing such a liposome or vesicle which method comprises at least the step of incorporating said fusion proteins into a liposome or vesicle and/or of forming a liposome or vesicle in the presence of said fusion proteins.

Such liposomes or vesicles that comprise such first and second fusion proteins can be as further described herein, and are preferably such that they have the chimeric GPCR of the invention suitably anchored or otherwise incorporated into the wall or membrane of the liposome or vesicle and spans said wall or membrane, more preferably such that at least one part of the amino acid sequence of the chimeric GPCR of the invention (and in particular, its extracellular binding site as defined herein) extends out (as defined herein) into the environment outside of the liposome or vesicle and at least one other part of the amino acid sequence of the chimeric GPCR of the invention (and in particular, its intracellular binding site as defined herein) extends out (as defined herein) into the environment inside the liposome or vesicle.

Said liposomes or vesicles are also preferably such that the first and second binding members of the binding pair can come into contact or in close proximity to each other when the second fusion protein binds (directly or indirectly, as described herein) to the chimeric GPCR of the invention that forms part of the first fusion protein. It will be clear to the skilled person that this generally means that the first and second binding members of the binding pair will be present in (as defined herein) the same environment relative to the wall or membrane of the liposome or vesicle. Preferably, the liposomes or vesicles are such that the first and second binding members of the binding pair will both be present in (as defined herein) the environment inside the liposome or vesicle.

Again, in the aspects of the invention that relate to liposomes or vesicles that contain such a first and second fusion protein, the chimeric GPCR of the invention, the protein that can bind directly or indirectly to the chimeric GPCR of the invention, the members of the binding pair and any linkers used can all be as further described herein.

In further aspects, the invention also relates to methods, and in particular assay methods or screening methods, that involve the use of the liposomes or vesicles described herein. As further described herein, such assay and screening methods can in particular be used to identify compounds and other chemical entities that bind to (and in particular specifically bind to) the chimeric GPCR of the invention and with that of the “first” GPCR from which the ECLs in said chimeric GPCR have been derived, that can modulate the translayer protein and said “first” GPCR and/or that modulate the signaling, signaling pathway and/or biological or physiological activitie(s) in which the first GPCR, its signaling and/or its signaling pathway is involved. As such, the liposomes or vesicles described herein can be used in methods to identify compounds or other chemical entities that can act as an agonist, antagonist, inverse agonist, inhibitor or modulator (such as an allosteric) modulator of the chimeric GPCR of the invention and of the first GPCR.

The invention also relates to uses of the liposomes or vesicles described herein, in particular in assay and screening methods and techniques. Such methods and uses can again be as further described herein for methods and uses of the arrangements of the invention.

Again, in all these aspects, such liposomes or vesicles and uses thereof are preferably as further described herein.

It will be clear to the skilled person that the compounds which are discovered, developed, generated and/or optimized using the methods and techniques which are described herein may be used for any suitable or desired purpose. Said purpose will generally be associated with the target against which the compounds have been screened/generated (i.e. the GPCR from which the ECLs have been derived that are present in the chimeric GPCR used), with the signaling, pathway(s) and/or mechanism of action with which the target is associated, and/or with the biological, physiological and/or pharmacological functions in which said target, pathway(s), signaling and/or mechanism of action are involved. Usually, and preferably, a compound of the invention will be such, and/or will be chosen such, that it is capable of modulating said target, signaling, pathway(s), mechanism of action and/or said biological, physiological and/or pharmacological functions in a desired or intended manner. As mentioned herein, this modulation can take any desired or intended form, including but not limited to upregulation and downregulation of the target, signaling, pathway(s), mechanism of action and/or said biological, physiological and/or pharmacological functions. As such, the compounds of the invention can for example function as agonists, antagonists, inverse agonists, inhibitors or another type of modulator (such as an allosteric modulator) for said target and/or its signaling, pathway(s), mechanism of action and/or said biological, physiological and/or pharmacological functions. All of this can be determined using suitable in vitro, cellular and/or in vivo assays (such as suitable efficacy or potency assays) and/or suitable animal models, depending on the specific target, signaling, pathway(s), mechanism of action and/or said biological, physiological and/or pharmacological functions involved. Suitable assays and models will be clear to the skilled person.

Usually, when a compound of the invention is an agonist (or antagonist, respectively) of the target, it will also be an agonist (or antagonist, respectively) of the signaling, pathway(s), mechanism of action and/or said biological, physiological and/or pharmacological functions in which the target is involved. However, as will be clear to the skilled person, it is also possible (and not excluded from the scope of the invention) that a compound of the invention may, for example and without being limited to any kind of hypothesis or explanation, be an agonist (or antagonist, respectively) of the target or its signaling but that such action as an agonist (or antagonist, respectively) of the target or its signaling results in an action as an antagonist (or antagonist, respectively) with respect to the biological, physiological and/or pharmacological functions in which the target or signaling is involved.

In one aspect of the practice of the invention, the arrangements and methods described herein will be used to test whether a compound or ligand that is present in environment [A] (for example, in the extracellular environment if the invention is performed in cells or the environment outside of the liposome or vesicle if the invention is performed in a liposome or vesicle) is capable of generating a detectable signal when it is contacted with the arrangement of the invention (i.e. in a way that allows said compound or ligand to bind to the binding site (8) on the chimeric GPCR of the invention). Similarly, when the methods and arrangements of the invention are used to screen a group, series or library of compounds or ligands, the methods and arrangements of the invention will be used to determine which compounds or ligands from said group, series or library generate a detectable signal (i.e. are “hits”).

Generally, in the invention, said detectable signal will be measured by measuring the signal that is (or can be) generated by the binding pair (6/7) (i.e. the signal that is generated when the first member (6) and the second member (7) come into contact with each other, come into close proximity to each other, or otherwise associate with each other to generate a detectable signal). It should be noted that, in the invention, usually a change in said signal is measured, and such change is also included within the term “generate a detectable signal” as used herein.

Said change can be either an increase in signal compared to a base level (which base level can also be below the detection limit of the equipment used to measure the signal, in which case there will be a signal detected in the presence of the compound of ligand where essentially no signal was measured before and this is also included within the term “increase in signal” as used herein) or a decrease in signal compared to a base level.

In the practice of the invention, an increase in signal will indicate that the compound or ligand acts as an agonist of the receptor. Conversely, a decrease in signal will indicate that the compound or ligand acts as an inverse agonist of the receptor. Thus, with advantage, the methods and arrangements of the invention may make it possible to identify both agonists and inverse agonists of a chimeric GPCR of the invention (and with that, agonists and antagonists of the GPCR from which the ECLs in said chimeric GPCR have been derived) and/or to distinguish agonists from inverse agonists (or visa versa).

It should be noted that the invention is not limited to any specific mechanism, explanation or hypothesis as to how the contact between the compound or ligand and (the binding site (8) on) the chimeric protein of the invention leads to a change in the detectable signal. However, it is assumed that one or more of the following mechanisms will be involved.

As mentioned herein, generally, the chimeric GPCR of the invention will, without the presence of the compound or ligand, exists in an equilibrium between two or more conformations, and some of these conformations will have low(er) affinity for (or even essentially no affinity for) the binding interaction between the (binding site (9) on) the chimeric protein of the invention and the second ligand (4) (i.e. the conformation-inducing binding domain or binding unit) compared to other conformations. Generally, in the invention, the level of detectable signal that is (or can be) measured at a certain point in time (or within a certain time interval) will depend on how much of the second ligand (4) (i.e. of the second fusion protein) binds or becomes bound to the chimeric GPCR of the invention, as the binding of the second fusion protein to the chimeric GPCR of the invention will bring (more of) the second binding member (7) in proximity to the first binding member (6), thus leading to the detectable signal (or an increase in the detectable signal compared to the level of background signal that may be present due to binding of “free” second ligand to the binding member (6), which background level is usually insignificant or below the detection limit).

Thus, generally, in the invention, a shift in the conformational equilibrium of the chimeric GPCR of the invention from states with low(er) or essentially no affinity for the second ligand (4) towards states with binding affinity for the second ligand (4) and/or states with better binding affinity for said second ligand (4) will generally lead to an increase in the detectable signal.

It is assumed that in the invention, the contacting of the chimeric GPCR of the invention with a compound or ligand that acts as an agonist will either shift this equilibrium towards conformational states with binding affinity for the second ligand (4) and/or states with better binding affinity, thus leading to an increase in signal that can be detected. This can for example be because the presence of the agonistic compound or ligand allows for the formation of new conformational states (for example, the formation of complexes comprising the compound or ligand, the chimeric GPCR of the invention and the second ligand) which cannot be formed when the compound or ligand is not present, because the agonistic compound or ligand stabilizes (or generally favors the formation of) conformational states that have high(er) affinity for the second ligand (4), and/or because the agonistic compound or ligand leads to new conformations that can bind the second ligand. Any one or more of these and other mechanisms (or any combination thereof) can be involved at any time, but the overall effect will be an increase of the amount of second ligand (4) that, at a certain moment in time (i.e. when the chimeric GPCR is in contact with the agonistic compound or ligand) and/or within a certain time interval (i.e. after the chimeric GPCR has been contacted with the agonistic compound or ligand), is associated with the chimeric GPCR and thus an increase in the amount of second binding member (7) that comes into contact or proximity to the first binding member (6) and thus to an increase in the detectable signal.

Based on the further description herein, it will also be clear to the skilled person that, because the chimeric GPCR exists in an equilibrium between states with no or low(er) affinity for the second ligand (2) and states with high(er) affinity for the second ligand (2), that even when the compound or ligand is not present, there will be a certain “basal” amount of the second fusion protein that is in contact with the second binding site (9) at any point in time of within a certain period of time. This basal level of binding will also lead to a certain basal level of detectable signal, which may be below the detection limit for the assay but in one specific aspect of the invention this basal signal is such that it is or can be detected (and/or the method of the invention is performed in such a way that it is detected). In such a case, an agonist will again lead to an increase in detectable signal compared to said basic level, but also an inverse agonist may shift the conformational equilibrium away from conformations with high(er) affinity for the second ligand (4) towards conformations with low(er) affinity for the second ligand (4). The result of this will be a decrease of the amount of second fusion protein that is bound to the chimeric GPCR at a certain moment in time and/or within a certain time interval, which will lead to a decrease in the detectable signal. Thus, this aspect and set-up of the invention will make it possible to screen for inverse agonists and/or to test compounds and ligands for activity as an inverse agonist. With advantage, this aspect and set-up of the invention will also make it possible to screen or test for agonists and antagonists as part of the same run of the screening or assay.

Again, the invention is not limited to any specific mechanism, explanation or hypothesis as to how a compound or ligand acts as an inverse agonist for the chimeric GPCR of the invention. However it is assumed that an inverse agonist may stabilize (or generally favor the formation of) conformational states that have low(er) affinity for the second ligand (4), may allow for the formation of new conformational states which cannot be formed when the compound or ligand is not present and which essentially cannot bind the second ligand (4) or only do so with low affinity, and/or may make it more difficult for the chimeric GPCR to undergo a conformational change into states that have higher affinity for the second ligand (4) (for example, by increasing the activation energy required for the conformational change). Any one or more of these and other mechanisms (or any combination thereof) can be involved at any time, but the overall effect will be a decrease in the amount of second ligand (4) that, at a certain moment in time (i.e. when the chimeric GPCR is in contact with the inverse agonist) and/or within a certain time interval (i.e. after the chimeric GPCR has been contacted with the inverse agonist), is associated with the chimeric GPCR and thus a decrease in the amount of second binding member (7) that is into contact or proximity to the first binding member (6) (compared to the situation where the inverse agonist is not present) and thus to a decrease in the detectable signal (i.e. compared to a basal signal without the presence of the inverse agonist).

Generally, the methods of the invention will comprise providing an arrangement as described herein and then contacting said arrangement with the compound(s) or ligand(s) to be screened or tested, i.e. for a certain period of time (which will usually be chosen so as to achieve a suitable or desired assay or screening “window”, and which may be benchmarked against a suitable window set with one or more known agonists or inverse agonists of the receptor involved) and in one or more concentrations, for example, to set a dose response curve and/or to allow the determination of an IC50 or another desired parameter (again, these concentrations may be chosen based on experience obtained with one or more known agonists or inverse agonists of the receptor involved). This will generally be performed using techniques for assay validation known per se.

The methods of the invention can be performed in a suitable medium, which may be water, a buffer or another suitable aqueous medium. When the methods of the invention are performed using cells or vesicles, the medium is preferably suitably chosen so as to ensure or promote viability of the cells or stability of the vesicles used, respectively.

After the arrangement of the invention has brought into contact with the compound(s) or ligand(s) to be screened or tested, the level of the detectable signal is measured at one or more moments in time or continuously over a desired time interval. This can be performed in any manner known per se, mainly depending on the binding pair (6/7) that is being used. Suitable equipment will be clear to the skilled person and will for example include the equipment used in the Experimental Section below. The value(s) obtained may also be compared to reference values (for example to the value(s) obtained in the same assay with one or more known agonists or inverse agonists, the value(s) obtained for a blank or carrier, and/or reference values obtained from previous experiments).

Based on the further disclosure herein, the skilled person will be able to suitably select other conditions (such as temperature) and equipment for performing the methods of the invention. Reference is also made to the Experimental Part herein for some suitable but non-limiting conditions.

For screening purposes, in particular of libraries of compounds or ligands, the methods of the invention may be performed in a high-throughput screening (HTS) format. When the methods of the invention are to be performed using cells, suitable techniques for performing cellular assays in HTS format can be applied. Reference is for example made to the review article by Rajalingham, BioTechnologia, 97(3), 227-234 (2016) and to Zang et al., International Journal of Biotechnology for Wellness Industries, 2012, 1, 31-51.

In the preceding paragraphs, the invention has been described with reference to FIG. 1, which shows an embodiment of the invention in which the second ligand (4) has been selected to bind directly to the binding site (9) on the chimeric GPCR of the invention. FIG. 2 shows an alternative embodiment of the invention, in which the second ligand (4) does not bind directly to the chimeric GPCR of the invention, but binds to another protein which other protein in turn can bind to the binding site (9) on the chimeric GPCR of the invention. In FIG. 2, said other protein (referred to herein for convenience as the “signaling protein”) is indicated as (5)—all other reference numbers in FIG. (2) are as defined herein for FIG. 1.

The overall principle of the embodiment shown in FIG. 2 is the same as for the method described herein for FIG. 1, in that the invention makes use of two fusion proteins that each comprise a member of the binding pair (6/7), and that binding of the first ligand (3) to the chimeric GPCR of the invention results in the first binding member (6) and the second binding member (7) of said binding pair coming into contact with, or in close proximity to, each other, giving rise to a detectable signal. Also, as with FIG. 1, and again without being limited to any specific mechanism, hypothesis or explanation, said signal will arise out of, increase or decrease as a result of, and/or otherwise be associated with a conformational change in the chimeric GPCR of the invention and/or a shift in the conformational equilibrium of the chimeric GPCR of the invention, essentially as described with respect to FIG. 1. However, in the embodiment of FIG. 2, said conformational change or shift in the conformational equilibrium will not be caused by (or associated with) the binding of the second ligand (4) to the chimeric GPCR of the invention, but instead by the binding of the signaling protein (5) to the chimeric GPCR. The second ligand (4) will bind to the signaling protein (5) when bound to the chimeric GPCR and so give rise to the detectable signal.

In this embodiment, again without being limited to any specific mechanism, hypothesis or explanation, it may be that the signaling protein (5) will only bind to those conformations of the chimeric GPCR of the invention that are associated with the binding of the first ligand (3) to the chimeric GPCR, so that the first and second binding member of the binding pair (6/7) can only come into contact or close proximity when the signaling protein (5) is bound to the chimeric GPCR. It is also possible that the signaling protein (5) itself undergoes a conformational change upon binding to the chimeric GPCR of the invention and that the second ligand (4) is selected such that it essentially only binds (or binds with higher affinity) to the conformation of the signaling protein (5) that arises upon binding to the chimeric GPCR of the invention. It is also possible that the signaling protein (5), upon binding to the chimeric GPCR of the invention, forms a complex with (or otherwise becomes associated with) other proteins, and that the second ligand (4) binds (or binds with higher affinity) to the complex.

However, notwithstanding to the foregoing, it will be clear to the skilled person based on the disclosure herein that the use of a chimeric GPCR in an arrangement as shown in FIG. 1 will usually be preferred, in particular in combination with the use of a conformation-inducing binding domain or binding unit as the second ligand (which then preferably forms part of the second fusion protein).

### EXPERIMENTAL PART

In the arrangements of the invention that are illustrated by Examples 1 to 3 below, a second fusion protein is used that binds indirectly to the relevant receptor. In said examples, the second fusion protein comprises either a VHH domain that binds to a G-protein complex (CA4435) or a VHH domain that binds to G-protein (CA4427).

In the arrangements of the invention that are illustrated by Examples 4 to 8 below, a second fusion protein is used that binds directly to the relevant receptor. In said examples, each of the second fusion proteins used comprises a VHH domain that binds to the G-protein binding site on the receptor used.

Table 1 below give the amino acid sequences of some of the fusion proteins, ConfoBodies and other elements referred to in the Examples below.

TABLE 1
amino acid sequences

| SEQ | | |
|---|---|---|
| ID | | |
| NO: | Description | Amino acid sequence |
| 1 | Hemagglutinin | MKTIIALSYIFCLVFA |
| | (HA) protein | |
| | signal | |
| | peptide | |
| 2 | FLAG-tag | DYKDDDDA |
| 3 | Linker | GAQGNS-GSSGGGGSGGGGSSG |
| 4 | Linker | GSSGGGGSGGGGSSG |
| 5 | large subunit | VFTLEDFVGDWEQTAAYNLDQVLEQGGVSSLLQNLAVSVTPIQRIVRSGENALKID |
| | of the NanoLuc | IHVIIPYEGLSADQMAQIEEVFKVVYPVDDHHFKVILPYGTLVIDGVTPNMLNYFGR |
| | luciferase | PYEGIAVFDGKKITVTGTLWNGNKIIDERLITPDGSMLFRVTINS |
| 6 | small subunit | VTGYRLFEEIL |
| | of the NanoLuc | |
| | luciferase | |
| 7 | XA8633 fusion | MQVQLQESGGGLVRPGGSRRLSCVDSERTSYPMGWFRRAPGKEREFVASITWSGI |
| | | DPTYADSVADRFTISRDVANNTLYLQMNSLKHEDTAVYYCAARAPVGQSSSPYDY |
| | | DYWGQGTQVTVSSGSSGGGGSGGGGSSGVTGYRLFEEIL |
| 8 | CA2780 fusion | MQVQLQESGGGLVQAGGSLRLSCAASGSIFSINTMGWYRQAPGKQRELVAAIHSG |
| | | GSTNYANSVKGRFTISRDNAANTVYLQMNSLKPEDTAVYYCNVKDYGAVLYEYD |
| | | YWGQGTQVTVSSGSSGGGGSGGGGSSGVTGYRLFEEIL |
| 9 | CA4437 fusion | MQVQLQESGGGFVQAGGSLRLSCAASGSIFSKNTMAWFRQAPGKERELVAASPTG |
| | | GSTAYKDSVKGRFTISRDSAKNTVLLQMNVLKPEDTAVYYCHLRQNNRGSWFHY |
| | | WGQGTQVTVSSGSSGGGGSGGGGSSGVTGYRLFEEIL |
| 10 | CA4435 fusion | MQVQLQESGGGLVQPGGSLRLSCAASGFTFSNYKMNWVRQAPGKGLEWVSDISQ |
| | | SGASISYTGSVKGRFTISRDNAKNTLYLQMNSLKPEDTAVYYCARCPAPFTRDCFD |
| | | VTSTTYAYRGQGTQVTVSSGSSGGGGSGGGGSSGVTGYRLFEEIL |
| 11 | MC4R-B2AR | MVNSTHRGMHTSLHLWNRSSYRLHSNASESLGKGYSDGGCYEQLFVSPEVFVTLG |
| | Chimer 1 | VISLLENILVITAIAKFERLQSPMYFFICSLAVADMLVSVSNGSETIVITLLNSTDTDA |
| | | QSFTVNIDNVIDSVICSSLLASICSLLSIAVDRYFAITSPFKYQSLLTVKRVGIIISCIWA |
| | | ACTVSGILFIIYSDSSAVIICLITMFFTMLALMASLYVRVFQEAKRQLQKIDKSEGRF |
| | | HVQNLSQVEQDGRTGHGLRRSSKFCLKEHKALKTLGIIIGVFVVCWAPFFLHLIFYI |
| | | SCPQNPYCVCFMSHFNLYLILIMCNSIIDPLIYCRSPDFRIAFQELLCLRR |
| 12 | MC4R-B2AR | MVNSTHRGMHTSLHLWNRSSYRLHSNASESLGKGYSDGGCYEQLFVSPEVFVTLG |
| | Chimer 2 | VISLLENILVITAIAKFERLQTVTNYFICSLAVADMLVSVSNGSETIVITLLNSTDTDA |
| | | QSFTVNIDNVIDSVICSSLLASICSLLSIAVDRYFAITSPFKYQSLLTVKRVGIIISCIWA |
| | | ACTVSGILFIIYSDSSAVIICLITMFFTMLALMASLYSRVFQEAKRQLQKIDKSEGRFH |
| | | VQNLSQVEQDGRTGHGLRRSSKFCLKEHKALKTLGILIGVFVVCWAPFFLHLIFYIS |
| | | CPQNPYCVCFMSHFNLYLILIMCNSIIDPLIYCRSPDFRIAFQELLCLRR |
| 13 | MC4R | MVNSTHRGMHTSLHLWNRSSYRLHSNASESLGKGYSDGGCYEQLFVSPEVFVTLG |
| | | VISLLENILVIVAIAKNKNLHSPMYFFICSLAVADMLVSVSNGSETIVITLLNSTDTD |
| | | AQSFTVNIDNVIDSVICSSLLASICSLLSIAVDRYFTIFYALQYHNIMTVKRVGIIISCI |
| | | WAACTVSGILFIIYSDSSAVIICLITMFFTMLALMASLYVHMFLMARLHIKRIAVLPG |
| | | TGAIRQGANMKGAITLTILIGVFVVCWAPFFLHLIFYISCPQNPYCVCFMSHFNLYLI |
| | | LIMCNSIIDPLIYALRSQELRKTFKEIICCYPLGGLCDLSSRY |
| 14 | B2AR | MGQPGNGSAFLLAPNGSHAPDHDVTQERDEVWVVGMGIVMSLIVLAIVFGNVLVI |
| | | TAIAKFERLQTVTNYFITSLACADLVMGLAVVPFGAAHILMKMWTFGNFWCEFWT |
| | | SIDVLCVTASIETLCVIAVDRYFAITSPFKYQSLLTKNKARVIILMVWIVSGLTSFLPI |
| | | QMHWYRATHQEAINCYANETCCDFFTNQAYAIASSIVSFYVPLVIMVFVYSRVFQE |
| | | AKRQLQKIDKSEGRFHVQNLSQVEQDGRTGHGLRRSSKFCLKEHKALKTLGIIMGT |
| | | FTLCWLPFFIVNIVHVIQDNLIRKEVYILLNWIGYVNSGFNPLIYCRSPDFRIAFQELL |
| | | CLRRSSLKAYGNGYSSNGNTGEQSGYHVEQEKENKLLCEDLPGTEDFVGHQGTVP |
| | | SDNIDSQGRNCSTNDSLL |
| 15 | APL receptor- | MKTIIALSYIFCLVFADYKDDDDAMEEGGDFDNYYGADNQSECEYTDWKSSGALI |
| | MOR chimer | PAIYMLVFLLGTTGNGLVLWTIVRYTKMKRRSADIFIASLAVADLTFVVTLPLWAT |
| | | YTYRDYDWPFGTFFCKLSSYLIFVNMYASVFCLTGLSFDRYLAICHPVKALDFRTP |
| | | RNGAVATAVLWVLAALLAMPVMVLRTTGDLENTTKVQCYMDYSMVATVSSEW |
| | | AWEVGLGVSSTTVGFVVPFTIMLTCYGLMILRLKSVRMLSGSKEKDRNLRRILSIIV |
| | | VLVVTFALCWMPYHLVKTLYMLGSLLHWPCDFDLFLMNIFPYCTCISYVNSCLNPF |
| | | LYAFLDENFKRCFREFCIPTSSNI |
| 16 | fusion protein | MKTIIALSYIFCLVFADYKDDDDAMEEGGDFDNYYGADNQSECEYTDWKSSGALI |
| | | PAIYMLVFLLGTTGNGLVLWTIVRYTKMKRRSADIFIASLAVADLTFVVTLPLWAT |
| | | YTYRDYDWPFGTFFCKLSSYLIFVNMYASVFCLTGLSFDRYLAICHPVKALDFRTP |
| | | RNGAVATAVLWVLAALLAMPVMVLRTTGDLENTTKVQCYMDYSMVATVSSEW |
| | | AWEVGLGVSSTTVGFVVPFTIMLTCYGLMILRLKSVRMLSGSKEKDRNLRRILSIIV |
| | | VLVVTFALCWMPYHLVKTLYMLGSLLHWPCDFDLFLMNIFPYCTCISYVNSCLNPF |
| | | LYAFLDENFKRCFREFCIPTSSNIGAQGNSGSSGGGGSGGGGSSGVFTLEDFVGDWE |
| | | QTAAYNLDQVLEQGGVSSLLQNLAVSVTPIQRIVRSGENALKIDIHVIIPYEGLSAD |
| | | QMAQIEEVFKVVYPVDDHHFKVILPYGTLVIDGVTPNMLNYFGRPYEGIAVFDGK |
| | | KITVTGTLWNGNKIIDERLITPDGSMLFRVTINS |
| 17 | UniProt P07550 | MGQPGNGSAFLLAPNGSHAPDHDVTQERDEVWVVGMGIVMSLIVLAIVFGNVLVI |
| | (ADRB2_HUMAN) | TAIAKFERLQTVTNYFITSLACADLVMGLAVVPFGAAHILMKMWTFGNFWCEFWT |
| | | SIDVLCVTASIETLCVIAVDRYFAITSPFKYQSLLTKNKARVIILMVWIVSGLTSFLPI |
| | | QMHWYRATHQEAINCYANETCCDFFTNQAYAIASSIVSFYVPLVIMVFVYSRVFQE |
| | | AKRQLQKIDKSEGRFHVQNLSQVEQDGRTGHGLRRSSKFCLKEHKALKTLGIIMGT |
| | | FTLCWLPFFIVNIVHVIQDNLIRKEVYILLNWIGYVNSGFNPLIYCRSPDFRIAFQELL |
| | | CLRRSSLKAYGNGYSSNGNTGEQSGYHVEQEKENKLLCEDLPGTEDFVGHQGTVP |
| | | SDNIDSQGRNCSTNDSLL |
| 18 | OX2-MOR chimer | MSGTKLEDSPPCRNWSSASELNETQEPFLNPTDYDDEEFLRYLWREYLHPKEYEW |
| | | VLIAGYIIVFVVALIGNVLVMYVIVRYTKMKTATNYFIVNLSLADVLVTITCLPATL |
| | | VVDITETWFFGQSLCKVIPYLQTVSVSVSVLTLSCIALDRYIAVCHPVKALDFRTPR |
| | | NARNSIVIIWIVSCIIMIPQAIVMECSTVFPGLANKTTLFTVCDERWGGEIYPKMYHI |
| | | CFFLVTYMAPLCLMVLAYGLMILRLKSVRMLSGSKEKDRARRKTARMLMIVLLVF |
| | | AICYLPISILNVLKRVFGMFAHTEDRETVYAWFTFSHWLVYANSAANPIIYAFLDEN |
| | | FKRCFREFCIPTSSNI |
| 19 | MOR (truncated) | APTNASNCTDALAYSSCSPAPSPGSWVNLSHLDGNLSDPCGPNRTDLGGRDSLCPPTGS |
| | UniProt | PSMITAITIMALYSIVCVVGLFGNFLVMYVIVRYTKMKTATNIYIFNLALADALATSTLP |
| | P35372-1 | FQSVNYLMGTWPFGTILCKIVISIDYYNMFTSIFTLCTMSVDRYIAVCHPVKALDFRTPR |
| | | NAKIINVCNWILSSAIGLPVMFMATTKYRQGSIDCTLTFSHPTWYWENLLKICVFIFAFI |
| | | MPVLIITVCYGLMILRLKSVRMLSGSKEKDRNLRRITRMVLVVVAVFIVCWTPIHIYVII |
| | | KALVTIPETTFQTVSWHFCIALGYTNSCLNPVLYAFLDENFKRCFREFCIPTSSNI |
| 20 | CA2780 | QVQLQESGGGLVQAGGSLRLSCAASGSIFSINTMGWYRQAPGKQRELVAAIHSGGSTN |
| | | YANSVKGRFTISRDNAANTVYLQMNSLKPEDTAVYYCNVKDYGAVLYEYDYWGQGT |
| | | QVTVSS |
| 21 | X8633 | QVQLVESGGGLVRPGGSRRLSCVDSERTSYPMGWFRRAPGKEREFVASITWSGIDPTY |
| | | ADSVADRFTISRDVANNTLYLQMNSLKHEDTAVYYCAARAPVGQSSSPYDYDYWGQ |
| | | GTQVTVSS |
| 22 | CA4435 | QVQLQESGGGLVQPGGSLRLSCAASGFTFSNYKMNWVRQAPGKGLEWVSDISQSGASI |
| | | SYTGSVKGRFTISRDNAKNTLYLQMNSLKPEDTAVYYCARCPAPFTRDCFDVTSTTYA |
| | | YRGQGTQVTVSS |
| 23 | CA4437 | QVQLQESGGGFVQAGGSLRLSCAASGSIFSKNTMAWFRQAPGKERELVAASPTGGSTA |
| | | YKDSVKGRFTISRDSAKNTVLLQMNVLKPEDTAVYYCHLRQNNRGSWFHYWGQGTQ |
| | | VTVSS |

### Example 1: Receptor Screening Assay Used in Examples 2 to 4

In the following Examples 2 to 4 a receptor screening assay is used that is essentially as described in Example 4 of the co-pending US provisional application filed on Apr. 29, 2019 and entitled “Screening methods and assays for use with transmembrane proteins, in particular with GPCRs”. Said assay is performed using an arrangement that is essentially as schematically shown in FIG. 1.

As described in said Example 4, the receptor screening assay is performed with Human Embryonic Kidney (HEK) 293T cells transiently transfected with a pBiT1.1C (Promega) expression vector encoding the relevant chimeric GPCR and with a pcDNA3.1 expression vector encoding a ConfoBody that is specific for the ICLs in the chimeric GPCR. [In Example 2, CA2780 (SEQ ID NO: 4 in WO 12/007593 and SEQ ID NO:20 herein) was used and in Examples 3 and 4 XA8633 (SEQ ID NO: 19 in WO14/118297 and SEQ ID NO:21 herein) was used. Alternatively, as described in the co-pending PCT application (although usually less preferred for use with a chimeric GPCR of the invention), a CA4437-SmBit fusion (SEQ ID NO:9) or a CA4435-SmBiT fusion (SEQ ID NO:10) or a biparatopic CA4435-35GS-CA4437-SSSmBiT fusion may be used.] The expression vector encoding the chimeric GPCR has a cleavable hemagglutinin (HA) protein signal peptide derived from influenza virus (MKTIIALSYIFCLVFA; SEQ ID NO: 1) followed by a FLAG-tag sequence (DYKDDDDA; SEQ ID NO:2) and is fused on the C-terminus via a flexible linker (GAQGNS-GSSGGGGSGGGGSSG; SEQ ID NO:3) to the large subunit of the NanoLuc luciferase (LgBit, SEQ ID NO:5). XA8633 is fused via a flexible linker (GSSGGGGSGGGGSSG; SEQ ID NO:4) on the C-terminus to the small subunit of the NanoLuc luciferase (SmBiT, SEQ ID NO:6).

HEK 293T cells are seeded in 6-well plate at 1 million cells per well and allowed to attach for at least 16 hours prior to transfection. HEK 293T cells are maintained at 37° C., 5% CO2, under humidified atmosphere in Dulbecco's Modified Eagle's Medium (DMEM) supplemented with 10% heat-inactivated FBS, 100 U/ml penicillin, 100 μg/ml streptomycin, 4 mM L-Glutamine and 1 mM sodium pyruvate (Gibco). MOR-LgBiT and XA8633-SmBiT are transfected using a 1:1 DNA ratio (corresponds to 1.5 μg for each construct) and X-tremeGENE HP DNA transfection reagent (Roche) was used for transfection using a 3 to 1 ratio of microliter transfection reagent volume to microgram DNA.

24 hours after transfection, the cells are harvested using culture medium and washed twice with Opti-MEM I reduced serum medium without phenol red (Gibco) to remove any remaining FBS. Transfected cells are seeded in white 96-well flat bottom tissue-culture treated plate (Corning; 3917) using a density of 50,000 cells per well (90 μl). After a 30 minutes incubation time at 37° C., 5% CO2, 20 μl of compound solution (agonist or antagonist) prepared as 5.5× stock solution in Opti-MEM is added to each well, gently mixed by hand and incubated for 1 hour at room temperature. Solvent controls were run in all experiments. Agonists DAMGO (Tocris, 1171), PZM21 (Medchemexpress, HY-101386), TRV130 (Advanced ChemBlocks, M15340), hydromorphone (Sigma Aldrich, H5136) and antagonist naloxone (Tocris, 599) are applied at different concentrations in the assay. The Nano-Glo® Live Cell Substrate (Promega) is diluted 20× in the Nano-Glo® LCS dilution buffer to make a 5× stock for addition to the cell culture medium. 25 μl of diluted Nano-Glo® substrate is added to each well, gently mixed by hand and luminescence is continuously monitored for 120 minutes (one measurement every 2 minutes) on Envision or SpectraMax i3x plate reader.

Curve fitting and statistical analysis is performed in GraphPad Prism and data are represented as mean Area Under the Curve (AUC) and standard error on the mean. 2-3 replicates are implemented per data point. Data is represented as normalized AUC which corresponds to the ratio AUC (sample) over AUC (blank).

### Example 2: Screening Assay for Recombinant MCR4

The receptor screening assay described in Example 1 was used. An expression vector encoding a recombinant MC4R receptor expression vector has a cleavable hemagglutinin (HA) protein signal peptide derived from influenza virus (MKTIIALSYIFCLVFA; SEQ ID NO: 1) followed by a FLAG-tag sequence (DYKDDDDA; SEQ ID NO:2) and is fused on the C-terminus via a flexible linker (GAQGNS-GSSGGGGSGGGGSSG; SEQ ID NO:3) to the large subunit of the NanoLuc luciferase (LgBit; SEQ ID NO:5). CA2780 (SEQ ID NO: 4 in WO 12/007593 and SEQ ID NO:20 herein) is fused via a flexible linker (GSSGGGGSGGGGSSG; SEQ ID NO:4) on the C-terminus to the small subunit of the NanoLuc luciferase (SmBiT; SEQ ID NO: 6). The ratio of DNA of the recombinant MC4R pBiT1.1C expressing vector and CA2780 pcDNA3.1 expressing vector during transfection was 1:1 (corresponds to 1.5 μg of each construct). Agonist NDP-alpha-MSH (Tocris, 3013), Rm-493 (Setmelanotide) (ChemScene LLC, CS-6399) and antagonist SHU9119 (Tocris, 3420) are applied at different concentrations in the assay. Samples and vehicle are prepared in Opti-MEM I reduced medium.

The sequence of the CA2780 fusion used in this Example is given in Table 1 as SEQ ID NO:8.

The results are shown in FIGS. 4 and 5. As can be seen, using the assay of this example, it was possible to distinguish agonists from the antagonists and the reference (blank) and to establish a dose response curve for one of the agonists.

### Example 3: Screening Assay for Recombinant OX2R

Essentially the same receptor screening assay as described in Example 1 was used, except for that a recombinant human OX2R receptor (SEQ ID NO:18) having the ECLs from OX2R and the ICLs from the μ-opioid receptor was expressed in pcDNA3.1 vector instead of pBiT1.1C. The recombinant OX2R expression vector has a cleavable hemagglutinin (HA) protein signal peptide derived from influenza virus (MKTIIALSYIFCLVFA; SEQ ID NO:1) followed by a FLAG-tag sequence (DYKDDDDK; SEQ ID NO:2) and is fused on the C-terminus via a flexible linker (GAQGNS-GSSGGGGSGGGGSSG; SEQ ID NO:3) to the large subunit of the NanoLuc luciferase (LgBit). XA8633 is fused via a flexible linker (GSSGGGGSGGGGSSG; SEQ ID NO:4) on the C-terminus to the small subunit of the NanoLuc luciferase (SmBiT; SEQ ID NO:6). The ratio of DNA of the recombinant OX2R expressing vector and XA8633 expressing vector during transfection was 1:30 (corresponds to 50 ng of recombinant OX2R expressing vector and 1.5 μg XA8633 expressing vector). Agonists Orexin B (Tocris, 1456), TAK-925 (Enamine), CS-5456 (ChemScene LLC) and YNT-185 (Enamine) and antagonist EMPA (Tocris, 4558) are applied at different concentrations in the assay. Samples and vehicle are prepared in Opti-MEM I reduced medium containing final 1% DMSO and 0.0015% Tween20.

The sequence of the XA8633 fusion used in this Example is given in Table 1 as SEQ ID NO: 7. For reference, the amino acid sequence of a human truncated (residues 6-360) μ-opioid receptor (UniProt P35372-1) is given in Table 1 as SEQ ID NO:19.

The results are shown in FIGS. 6 to 10. As can be seen, using the assay of this example, it was possible to distinguish agonists from the antagonists and to establish dose response curves for the agonists.

### Example 4: Screening Assay for Recombinant APJ Receptor

The receptor screening assay described in Example 1 was used. The chimeric GPCR was an apelin/mu-opioid receptor chimer with the ECLs from apelin and the ICLs from the mu-opioid receptor. Its full sequence is given as SEQ ID NO: 15. pcDNA3.1 expression vector encoding a recombinant human APJ receptor has a cleavable hemagglutinin (HA) protein signal peptide derived from influenza virus (MKTIIALSYIFCLVFA; SEQ ID NO: 1) followed by a FLAG-tag sequence (DYKDDDDA; SEQ ID NO:2) and is fused on the C-terminus via a flexible linker (GAQGNS-GSSGGGGSGGGGSSG; SEQ ID NO:3) to the large subunit of the NanoLuc luciferase (LgBit; SEQ ID NO:5). XA8633 is fused via a flexible linker (GSSGGGGSGGGGSSG; SEQ ID NO:4) on the C-terminus to the small subunit of the NanoLuc luciferase (SmBiT). The sequence of the resulting fusion protein is given as SEQ ID NO:16. The ratio of DNA of the recombinant APJ receptor expressing pcDNA3.1 vector and XA8633 expressing pcDNA3.1 vector during transfection was 1:150 (corresponds to 10 ng of the recombinant APJ receptor expressing vector and 1.5 μg of the XA8633 expressing vector). Agonists [Pyr1]-Apelin-13 (Tocris, 2420), ELA-14 (Tocris, 6293), CMF-019 (Aobious, AOB8242) and antagonist MM 54 (Tocris, 5992) are applied at one or two concentrations in the assay. Samples and vehicle are prepared in Opti-MEM I reduced medium containing final 1% DMSO and 0.0015% Tween20.

The sequence of the XA8633 fusion used in this Example is given in Table 1 as SEQ ID NO: 7.

The results are shown in FIG. 11A. As can be seen, using the assay of this example, it was possible to distinguish strong agonists from weaker agonists and from the antagonist and the reference (blank).

In a separate experiment, instead of an apelin-mu-opioid receptor chimer, a apelin-beta-2AR receptor chimer with the ECLs from the apelin receptor and the ICLs from the beta-2AR receptor was used in an assay of the invention. The other fusion protein used was a CA2780-SmBiT fusion. The results are shown in FIG. 11B. In addition to the agonists [Pyr1]-Apelin-13 (Tocris, 2420), ELA-14 (Tocris, 6293), CMF-019 (Aobious, AOB8242) and antagonist MM-54 (Tocris, 5992), one further known APJ agonist (MM-07, Tocris, 7053) was tested. As can be seen, also when this other chimer was used in an assay of the invention, it was possible to distinguish strong agonists of the apelin receptor from weaker agonists and from the antagonist and the reference (blank), even if the assay window was not be exactly the same as the assay window of the assay used in FIG. 11A.

### Example 5: Screening of Compound Libraries

A library of 80 compounds (arrayed on a 96 well plate with 16 references) was screened using the assay essentially described in Example 3. Cells were suspended in Opti-MEM and the compounds were added in Opti-MEM plus 0.0015% Tween. The cells were allowed to stabilize for 1 hour at room temperature after which NanoGlo was added (30 minutes at room temperature) followed by the compound to be tested (30 or 60 minutes).

The screening results are shown in FIG. 12 (control plate) and 13 (screening plate). The data from the control plate confirms that the assay can distinguish a known agonist for OX2R (TAK925) from a reference (blank). The data for the screening plate shows that the screening assay is also capable of identifying hits from a library of compounds of unknown activity with respect to OX2R.

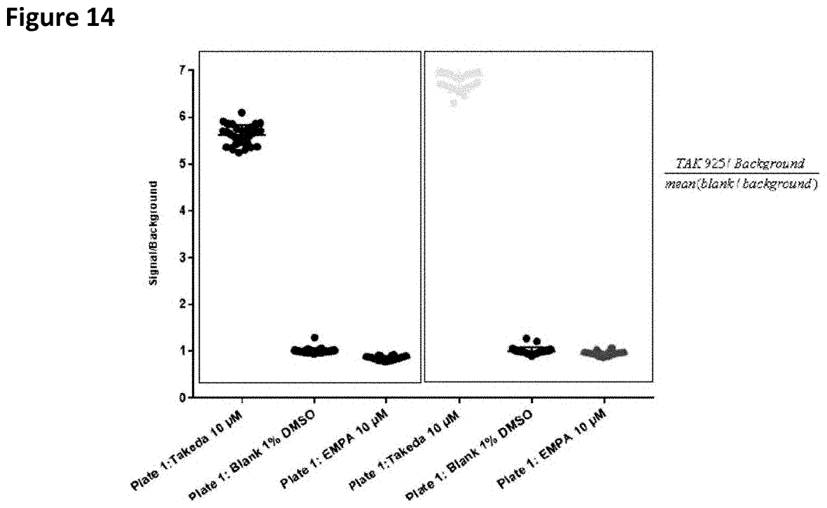

The same assay was used to perform a second screening run on a different library of 80 compounds (again, arrayed on a 96 well plate with 16 references) compounds. The results are shown in FIG. 14 (control plate) and 15 (screening plate).

### Example 6: Single Point Radioligand Assay

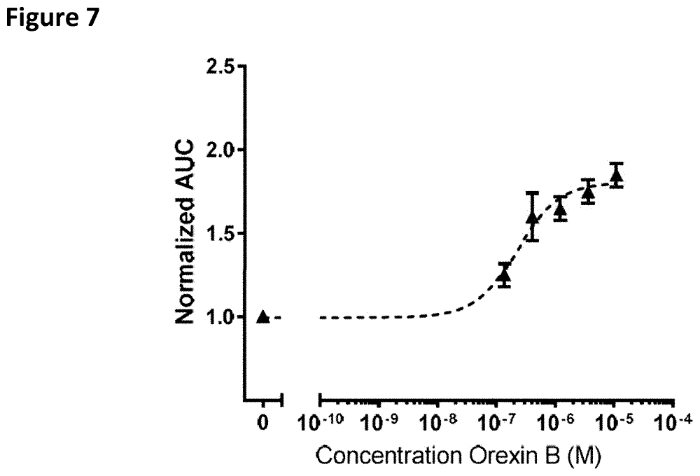

MC4R-B2AR chimer (SEQ ID NO: 11, see also FIGS. 16 and 7) and wild-type MC4R (SEQ ID NO: 13) were tested in a single point radioligand assay against a series of chemical compounds (indicated on the x-axis in FIG. 18 as “A2” to “F11”).

The radioligand assay was performed in 96-well flat-bottom plates using a Perkin-Elmer MicroBeta plate reader. Each sample had a total volume of 100 microliter. For the wild-type MC4R, the sample had the following composition: 10 microgram MC4R protein, added to the sample as 20 microliter of a radiolabeled membrane extract; 20 microliter of radioligand solution ([125I]-SHU-9119) to give a final concentration of radioligand in the sample of 0.1 nM; 60 microliter of assay buffer (25 mM HEPES, 100 mM NaCl, 0.20% BSA, 50 micromolar GTPgS, pH 7.4); and 1 microliter of a buffer solution of the compound be tested so as to give a final concentration of the compound in the sample of 10 micromolar. For the chimer, the sample had the following composition: 5 microgram of chimer protein, added to the sample as 20 microliter of a radiolabeled membrane extract; 20 microliter of radioligand solution ([125I]-SHU-9119) at a final concentration of radioligand in the sample of 0.1 nM; 60 microliter of assay buffer (25 mM HEPES, 0.1 mM MgCl2, 1 mM CaCl₂, 0.20% BSA, 50 micromolar GTPgS, pH 7.4); and 1 microliter of a buffer solution of the compound be tested so as to give a final concentration of the compound in the sample of 10 micromolar. Reference samples for non-specific binding and a positive control were also included in the plates.

Each sample was incubated at room temperature for 60 min after which the reaction was terminated by rapid vacuum filtration onto glass fiber filter. CPMs were counted using a scintillation counter.

The results are schematically shown in FIG. 18. For each compound, displacement with respect to wildtype is indicated with a dot and displacement with respect to the chimer is indicated with a square, allowing direct comparison of displacement on wild-type and chimer for each compound.

As can be seen from FIG. 18:
some of the compounds tested (for example A2, C3 and G2) led to the displacement of the ligand for the both the wildtype and the chimer. In some cases (such as C2, B5 and C5), the displacement provided was essentially similar;
other compounds tested (for example A4, E4 and H4) essentially led to no displacement of the ligand for both the wildtype and the chimer (or a displacement that was essentially below the cut-off for the radioligand assay).

These results confirm that the chimer is functional in the radioligand assay and that its functionality is comparable to, and representative for, that of the wildtype. However, FIG. 18 also demonstrates an advantage of the use of the chimer in this set-up. Namely, for some of the compounds (such as H2, A3, C9, D9 and E9) the displacement of ligand on the chimer was much greater the displacement of ligand on the wild-type. In the set-up used in this Example 6, this identifies said compounds as potential agonists (i.e. for the chimer and thus also for a functional state of the wildtype). Also, some compounds (like C2, B5 and C5) provided essentially similar displacement (i.e. above the cut-off) for both the chimer and the wildtype in this assay. This identifies such compounds as potential antagonists.

### Example 7: Conformation of Hits Identified Using a Chimer in a Cellular Assay Using the Wild-Type Receptor

A series of chemical compounds were tested for activity in an assay set up according to Example 1 and in a radioligand assay

The results are shown in FIG. 19, in which for each compound (indicated with a dot in FIG. 19), the values obtained from the radioligand assay are plotted along the y-axis (“Conforatio@10 micromolar”) and results from the assay of Example 1 are set out along the x-axis (“ConfoSensor ratio”). The results show that a positive signal in the assay set up of Example 1 (“confosensor ratio”>1.2) is indicative of a positive value in radioligand (“conforatio”>6).

Based on these results, a number of compounds (indicated as A to I in FIG. 19) were tested in a standard cAMP cellular assay to confirm whether they are agonists of wild-type MC4R. Alpha-MSH was included as a positive control. The results are shown in FIGS. 20 and 21 (which show the results of two separate experiments).

As can be seen, a number of the compounds identified using the chimer could be confirmed as being agonists of the wild-type MC4R. The results also show that compound H (which did not give a positive signal in either radioligand or the set-up of Example 1) essentially did not show agonist activity under the conditions used.

### Example 8: Comparison of an Assay Using a GPCR Chimer and a cAMP Assay (HTRF)

Two assays for testing compounds directed to MC4R were compared: (i) a conventional homogeneous time resolved fluorescence (HTRF) cyclic AMP assay; and (ii) an assay using a GPCR-LgBiT fusion (in which the GPCR was a recombinant GPCR of the invention essentially having the ECLs and TMs from MC4R and the ICLs for beta-2AR) and a CA2780-SmBiT fusion.

Using these assays, the IC50 (for the cAMP HTRF assay) and EC50 values (assay using a chimer of the invention) were determined for 5 compounds known to modulate MC4R as well as for a-MSH (reference). The results are listed in Table 2, with the two compounds that performed best in the cAMP assay also performing best in the assay using the chimer of the invention and the compound that performed worse in the cAMP assay also performing worse in the assay using the chimer of the invention.

The assays were performed as follows: The measurement of the accumulation of 3′,5′-cyclic adenosine monophosphate (cAMP) in intact CHO cells stably expressing human WT MC4R was performed using a LANCE® Ultra cAMP Kit (Perkin Elmer) according to manufacturer recommendations. Measurement of the signal was performed using Envision plate reader.

For the assay with recombinant MC4R-LgBiT fusion the same process was used as in Example 2. pcDNA3.1 expression vector encoding recombinant MC4R has a cleavable hemagglutinin (HA) protein signal peptide derived from influenza virus (MKTIIALSYIFCLVFA; SEQ ID NO:1) followed by a FLAG-tag sequence (DYKDDDDA; SEQ ID NO:2) and is fused on the C-terminus via a flexible linker (GAQGNS-GSSGGGGSGGGGSSG; SEQ ID NO:3) to the large subunit of the NanoLuc luciferase (LgBit; SEQ ID NO: 5). CA2780 (SEQ ID NO: 4 in WO 12/007593 and SEQ ID NO: 20 herein) is fused via a flexible linker (GSSGGGGSGGGGSSG; SEQ ID NO:4) on the C-terminus to the small subunit of the NanoLuc luciferase (SmBiT; SEQ ID NO:6). The ratio of DNA of the recombinant MC4R pcDNA3.1 expressing vector and CA2780 pcDNA3.1 expressing vector during transfection was 1:100 (corresponds to 0.015 μg of recombinant MC4R expressing vector and 1.5 μg of CA2780 expressing vector).

Agonist alpha-MSH (Tocris, 2584) and 5 compounds known to modulate MC4R are applied at different concentrations in both assays. Samples and vehicle are prepared in Opti-MEM I reduced medium containing final 1% DMSO and 0.00022% Tween20. The cells were allowed to stabilize for 1 hour at room temperature after which NanoGlo was added (30 minutes at room temperature) followed by the compound to be tested (30 or 45 minutes). Luminescence is measured on Envision plate reader.

TABLE 2
Results from comparison of an assay using a
chimer of the invention and a cAMP assay.

| Compound | cAMP (IC50) | Chimer (EC50) |
|---|---|---|
| 2-A | 8.00E−07 | 8.38E−09 |
| 2-B | 5.44E−07 | 3.26E−09 |
| a-MSH | 1.61E−07 | 1.48E−08 |
| 2-C | 4.45E−06 | 5.46E−08 |
| 2-D | 3.43E−06 | 8.30E−08 |
| 2-E | 1.17E−05 | 1.32E−07 |

### Example 9: Screening of Compound Library

A plate of small chemical compounds (fragment library) was screened in an assay of the invention using a recombinant OX2R-MOR chimer (SEQ ID NO:18) fused to LgBiT and XA8633 (SEQ ID NO: 21) fused to SmBiT (see Example 3) and in a commercially available OX2 IP-One assay.

The results are plotted in FIG. 23, with the x-axis representing the data obtained in the assay of the invention, the y-axis representing the data obtained in the IP-One assay and each dot representing the results for a single compound. As can be seen, there was a reasonable degree of correlation between the results obtained using the assay of the invention and the results obtained in the IP-One assay. A similar degree of correlation was observed when the results obtained using the same assay of the invention were compared to the results obtained in an OX2 radioligand assay (data not shown).

### Example 10: Screening of Large Compound Library

A library of 11378 compounds was screened in an assay of the invention using a recombinant OX2R-MOR chimer (SEQ ID NO:18) fused to LgBiT and XA8633 (SEQ ID NO: 21) fused to SmBiT (see Example 3).

The results are plotted in FIG. 24A (compounds tested at 30 μM) and 24B (compounds tested at 200 μM), with the x-axis representing the ratio of the signal obtained with the compound tested (“sample”) vs signal given by the carrier solvent (“blank”) and each dot representing the result obtained for a single compound.

As can be seen from these plots, screening of the large compound library using the assay of the invention afforded multiple hits.

1. Chimeric GPCR having the structure:
[N-terminal sequence]-[TM1]-[IC1]-[TM2]-[EC1]-[TM3]-[IC2]-[TM4]-[EC2]-[TM5]-[IC3]-[TM6]-[EC3]-[TM7]-[C-terminal sequence]
in which the extracellular loops are derived from a first GPCR and the intracellular loops that are derived from a second GPCR (different from the first).

2. Chimeric GCPR according to claim 1, in which the extracellular binding domain of said chimeric GPCR is derived from said first GPCR.

3. Chimeric GCPR according to claim 1, in which the TMs are derived from said first GPCR.

4. Chimeric GCPR according to claim 1, in which the first GPCR and the second GPCR both belong to class A.

5. Composition comprising a chimeric GPCR according to claim 1 and a binding domain or binding unit that can bind to at least one of the intracellular loops that are derived from said second GPCR.

6. Composition according to claim 5, in which said binding domain or binding unit is ligand is capable of stabilizing and/or inducing a functional and/or active conformational state of the chimeric GPCR upon binding to the chimeric GPCR.

7. Composition according to claim 5, in which said binding domain or binding unit is an immunoglobulin single variable domain.

8. Composition according to claim 5, which is a cellular composition.

9-11. (canceled)

12. Arrangement that comprises at least the following elements:
a boundary layer that separates a first environment from a second environment;
a chimeric GPCR according to claim 1;
a first ligand for the chimeric GPCR that is present in the first environment;
a second ligand for the chimeric GPCR that is present in the second environment, which second ligand is a binding domain or binding unit that can bind to at least one of the intracellular loops on said chimeric GPCR; and
a binding pair that consists of at least a first binding member and a second binding member, which binding pair is capable of generating a detectable signal.

13. Arrangement according to claim 12, in which the chimeric GPCR is fused or linked, either directly or via a suitable spacer or linker, to the first binding member of said binding pair and in which the second ligand is fused or linked, either directly or via a suitable spacer or linker, to the first binding member of said binding pair.

14. The chimeric GPCR according to claim 1, wherein the chimeric GPCR is fused or linked, either directly or via a suitable spacer or linker, to a binding domain or binding unit that can bind to at least one of the intracellular loops of said chimeric GPCR.

15. Fusion protein according to claim 14, in which said binding domain or binding unit is ligand is capable of stabilizing and/or inducing a functional and/or active conformational state of the chimeric GPCR upon binding to the chimeric GPCR.


SEQUENCE LISTING (23 sequences)

SEQ ID NO: 1 - protein, 16 aa, Artificial sequence
  Hemagglutinin (HA) protein signal peptide
MKTIIALSYIFCLVFA

SEQ ID NO: 2 - protein, 8 aa, Artificial sequence
  FLAG-tag
DYKDDDDA

SEQ ID NO: 3 - protein, 21 aa, Artificial sequence
  Linker
GAQGNSGSSGGGGSGGGGSSG

SEQ ID NO: 4 - protein, 15 aa, Artificial sequence
  Linker
GSSGGGGSGGGGSSG

SEQ ID NO: 5 - protein, 158 aa, Artificial sequence
  Large subunit of the NanoLuc luciferase (LgBit)
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 6 - protein, 11 aa, Artificial sequence
  Small subunit of the NanoLuc luciferase (SmBit)
VTGYRLFEEIL

SEQ ID NO: 7 - protein, 149 aa, Artificial sequence
  XA8633 fusion
MQVQLQESGGGLVRPGGSRRLSCVDSERTSYPMGWFRRAPGKEREFVASITWSGIDPTYADSVADRFTISRDVANNTLYLQMNSLKHEDTAVYYCAARAPVGQSSSPYDYDYWGQGTQVTVSSGSSGGGGSGGGGSSGVTGYRLFEEIL

SEQ ID NO: 8 - protein, 147 aa, Artificial sequence
  CA2780 fusion
MQVQLQESGGGLVQAGGSLRLSCAASGSIFSINTMGWYRQAPGKQRELVAAIHSGGSTNYANSVKGRFTISRDNAANTVYLQMNSLKPEDTAVYYCNVKDYGAVLYEYDYWGQGTQVTVSSGSSGGGGSGGGGSSGVTGYRLFEEIL

SEQ ID NO: 9 - protein, 146 aa, Artificial sequence
  CA4437 fusion
MQVQLQESGGGFVQAGGSLRLSCAASGSIFSKNTMAWFRQAPGKERELVAASPTGGSTAYKDSVKGRFTISRDSAKNTVLLQMNVLKPEDTAVYYCHLRQNNRGSWFHYWGQGTQVTVSSGSSGGGGSGGGGSSGVTGYRLFEEIL

SEQ ID NO: 10 - protein, 155 aa, Artificial sequence
  CA4435 fusion
MQVQLQESGGGLVQPGGSLRLSCAASGFTFSNYKMNWVRQAPGKGLEWVSDISQSGASISYTGSVKGRFTISRDNAKNTLYLQMNSLKPEDTAVYYCARCPAPFTRDCFDVTSTTYAYRGQGTQVTVSSGSSGGGGSGGGGSSGVTGYRLFEEIL

SEQ ID NO: 11 - protein, 341 aa, Artificial sequence
  MC4R-B2AR chimer 1
MVNSTHRGMHTSLHLWNRSSYRLHSNASESLGKGYSDGGCYEQLFVSPEVFVTLGVISLLENILVITAIAKFERLQSPMYFFICSLAVADMLVSVSNGSETIVITLLNSTDTDAQSFTVNIDNVIDSVICSSLLASICSLLSIAVDRYFAITSPFKYQSLLTVKRVGIIISCIWAACTVSGILFIIYSDSSAVIICLITMFFTMLALMASLYVRVFQEAKRQLQKIDKSEGRFHVQNLSQVEQDGRTGHGLRRSSKFCLKEHKALKTLGIIIGVFVVCWAPFFLHLIFYISCPQNPYCVCFMSHFNLYLILIMCNSIIDPLIYCRSPDFRIAFQELLCLRR

SEQ ID NO: 12 - protein, 341 aa, Artificial sequence
  MC4R-B2AR chimer 2
MVNSTHRGMHTSLHLWNRSSYRLHSNASESLGKGYSDGGCYEQLFVSPEVFVTLGVISLLENILVITAIAKFERLQTVTNYFICSLAVADMLVSVSNGSETIVITLLNSTDTDAQSFTVNIDNVIDSVICSSLLASICSLLSIAVDRYFAITSPFKYQSLLTVKRVGIIISCIWAACTVSGILFIIYSDSSAVIICLITMFFTMLALMASLYSRVFQEAKRQLQKIDKSEGRFHVQNLSQVEQDGRTGHGLRRSSKFCLKEHKALKTLGILIGVFVVCWAPFFLHLIFYISCPQNPYCVCFMSHFNLYLILIMCNSIIDPLIYCRSPDFRIAFQELLCLRR

SEQ ID NO: 13 - protein, 332 aa, Artificial sequence [sequence omitted]

SEQ ID NO: 14 - protein, 413 aa, Artificial sequence [sequence omitted]

SEQ ID NO: 15 - protein, 356 aa, Artificial sequence
  APL receptor-MOR chimer
MKTIIALSYIFCLVFADYKDDDDAMEEGGDFDNYYGADNQSECEYTDWKSSGALIPAIYMLVFLLGTTGNGLVLWTIVRYTKMKRRSADIFIASLAVADLTFVVTLPLWATYTYRDYDWPFGTFFCKLSSYLIFVNMYASVFCLTGLSFDRYLAICHPVKALDFRTPRNGAVATAVLWVLAALLAMPVMVLRTTGDLENTTKVQCYMDYSMVATVSSEWAWEVGLGVSSTTVGFVVPFTIMLTCYGLMILRLKSVRMLSGSKEKDRNLRRILSIIVVLVVTFALCWMPYHLVKTLYMLGSLLHWPCDFDLFLMNIFPYCTCISYVNSCLNPFLYAFLDENFKRCFREFCIPTSSNI

SEQ ID NO: 16 - protein, 535 aa, Artificial sequence
  Fusion protein
MKTIIALSYIFCLVFADYKDDDDAMEEGGDFDNYYGADNQSECEYTDWKSSGALIPAIYMLVFLLGTTGNGLVLWTIVRYTKMKRRSADIFIASLAVADLTFVVTLPLWATYTYRDYDWPFGTFFCKLSSYLIFVNMYASVFCLTGLSFDRYLAICHPVKALDFRTPRNGAVATAVLWVLAALLAMPVMVLRTTGDLENTTKVQCYMDYSMVATVSSEWAWEVGLGVSSTTVGFVVPFTIMLTCYGLMILRLKSVRMLSGSKEKDRNLRRILSIIVVLVVTFALCWMPYHLVKTLYMLGSLLHWPCDFDLFLMNIFPYCTCISYVNSCLNPFLYAFLDENFKRCFREFCIPTSSNIGAQGNSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 17 - protein, 413 aa, Artificial sequence [sequence omitted]

SEQ ID NO: 18 - protein, 355 aa, Artificial sequence
  OX2-MOR chimer
MSGTKLEDSPPCRNWSSASELNETQEPFLNPTDYDDEEFLRYLWREYLHPKEYEWVLIAGYIIVFVVALIGNVLVMYVIVRYTKMKTATNYFIVNLSLADVLVTITCLPATLVVDITETWFFGQSLCKVIPYLQTVSVSVSVLTLSCIALDRYIAVCHPVKALDFRTPRNARNSIVIIWIVSCIIMIPQAIVMECSTVFPGLANKTTLFTVCDERWGGEIYPKMYHICFFLVTYMAPLCLMVLAYGLMILRLKSVRMLSGSKEKDRARRKTARMLMIVLLVFAICYLPISILNVLKRVFGMFAHTEDRETVYAWFTFSHWLVYANSAANPIIYAFLDENFKRCFREFCIPTSSNI

SEQ ID NO: 19 - protein, 355 aa, Artificial sequence
  MOR (truncated) Uniprot P35372-1
APTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNI

SEQ ID NO: 20 - protein, 120 aa, Artificial sequence
  CA2780
QVQLQESGGGLVQAGGSLRLSCAASGSIFSINTMGWYRQAPGKQRELVAAIHSGGSTNYANSVKGRFTISRDNAANTVYLQMNSLKPEDTAVYYCNVKDYGAVLYEYDYWGQGTQVTVSS

SEQ ID NO: 21 - protein, 122 aa, Artificial sequence
  X8633
QVQLVESGGGLVRPGGSRRLSCVDSERTSYPMGWFRRAPGKEREFVASITWSGIDPTYADSVADRFTISRDVANNTLYLQMNSLKHEDTAVYYCAARAPVGQSSSPYDYDYWGQGTQVTVSS

SEQ ID NO: 22 - protein, 128 aa, Artificial sequence
  CA4435
QVQLQESGGGLVQPGGSLRLSCAASGFTFSNYKMNWVRQAPGKGLEWVSDISQSGASISYTGSVKGRFTISRDNAKNTLYLQMNSLKPEDTAVYYCARCPAPFTRDCFDVTSTTYAYRGQGTQVTVSS

SEQ ID NO: 23 - protein, 119 aa, Artificial sequence
  CA4437
QVQLQESGGGFVQAGGSLRLSCAASGSIFSKNTMAWFRQAPGKERELVAASPTGGSTAYKDSVKGRFTISRDSAKNTVLLQMNVLKPEDTAVYYCHLRQNNRGSWFHYWGQGTQVTVSS